(19) United States
(12) Patent Application Publication
(10) Pub. No.: US 2025/0218568 A1
(43) Pub. Date: Jul. 3, 2025

(54) EARLY DETECTION TOOLS FOR MENTAL HEALTH

(71) Applicant: Pandora Bio, Inc., Sunnyvale, CA (US)

(72) Inventors: Tara Maddala, Sunnyvale, CA (US); Megan Rothney, San Francisco, CA (US); Jason Carlson, San Jose, CA (US); Akhil Nistala, San Francisco, CA (US); Miles Waugh, Sunnyvale, CA (US); Daniel Civello, Alameda, CA (US); Jennifer Geis, South San Francisco, CA (US)

(21) Appl. No.: 19/003,243

(22) Filed: Dec. 27, 2024

Related U.S. Application Data
(60) Provisional application No. 63/616,140, filed on Dec. 29, 2023.

(51) Int. Cl. G16H 20/70; G16H 40/67

(52) U.S. Cl. CPC G16H 20/70; G16H 40/67

(57) ABSTRACT

Provided herein are methods and systems for collecting data, analyzing data, and generating a mental health status or trajectory from the data or information derived from the data collected.

### RELATED APPLICATIONS

This application claims benefit of U.S. provisional patent application No. 63/616,140, filed on Dec. 29, 2023, which is incorporated herein by reference in its entirety.

### BACKGROUND

The mental health crisis among young people is worsening, with 60% of U.S. college students meeting criteria for mental health problems, influenced by factors like academic pressure and social isolation. However, only 40% seek help, partly due to perceived resource limitations. COVID-19 has exacerbated this crisis, leading to a rise in disorders such as depression, anxiety, substance use, behavioral disorders, and eating disorders, with women and Black students at increased risk. Colleges and secondary schools in particular need to prioritize mental health support and destigmatize seeking help, despite being under-resourced and facing pandemic-related challenges. There is a need for monitoring and interventions for improving mental health of students and young people, including college and high school students. There is also a need for monitoring and interventions for improving mental health of other groups, such as businesses, military units, and government organizations.

### SUMMARY

The disclosure provides a computer implemented method of training a model. The model may be trained to assess a mental health status or a change thereof of a user. The method may include collecting marker values of a population of test users. Markers may be from two or more data channels. Examples of data channels include passive data channels, active data channels, self-reported data channels, and external data channels. The method may include extracting a set of features from the marker values. The method may include training a model using the set of features. The model may be trained to assesses a mental health status based on the set of features.

The disclosure provides a computer implemented method of assessing a mental health status or a change thereof of a user. The method may include collecting marker values of the user from the two or more data channels. The method may include using a model trained as described herein to assess a mental health status of the user.

The disclosure provides a computer implemented method of extracting a health conclusion from a user's voice. The method may include collecting a first instance of a user's voice data. The method may include collecting a second instance of a user's voice data. Voice data may include a vocal cord characteristic, a speech characteristic, or a background noise characteristic. The method may include using a model to draw a health conclusion based on characteristics collected from the first and second instances. Voice data may be recorded by the user. Voice may be streamed by the user. Voice characteristics may include at least one of tone of voice, inflection of voice, word count, speech rate, intensity of voice, pitch, magnitude, phonetics, tempo-spectral, formant, glottal closure instance, or any combinations thereof.

The disclosure provides a computer implemented method of extracting a health conclusion from device usage data. The method may include collecting a user's device usage data at one or more points in time. The method may include using a model to draw a health conclusion based on the device usage data. Device usage data may include total time a user spent on a device. Device usage data may include total time using one or more specific apps. Device usage data may include total time using one or more categories of apps. The categories may include any one of social, entertainment, educational, and informational.

The disclosure provides a computer implemented method of extracting a health conclusion from a user's device. The method may include collecting from a device at multiple points in time, data on a user's positioning, voice, and device usage. The method may include using a model to draw a health conclusion based on the collected data.

The disclosure provides a computer implemented method of providing health information for a user. The method may include collecting data about a person. The method may include using a model to draw a health conclusion based on the collected data. The method may include providing at least one health resource option based on the health conclusion. Data may be self-reported. The self-reported data may be private. The self-reported data may be encoded.

The disclosure provides a computer implemented method of training a model for generating a health conclusion from location data. The method may include collecting location data on a user at one or more points in time. The method may include collecting data on a local condition at the user's position(s). The method may include extracting marker values from the location data and local-conditions data. The method may include training a model to generate a health conclusion based on marker values. The disclosure provides a computer implemented method of training model to generate a health conclusion from a user's voice. The method may include collecting from a first instance of a user's voice at least one of a vocal cord characteristic, a speech characteristic, and a background noise characteristic. The method may include collecting from a second instance of a user's voice at least one of a vocal cord characteristic, a speech characteristic, and a background noise characteristic. The method may include training a model to generate a health conclusion based on characteristics collected from the first and second recordings.

The disclosure provides a computer implemented method of training a model to generate a health conclusion from device usage data. The method may include collecting a user's device usage data at one or more points in time. The method may include training a model to generate a health conclusion based on the device usage data.

The disclosure provides a computer implemented method of training a model to generate a health conclusion from a user's device. The method may include collecting from a device at multiple points in time, data on a user's positioning, voice, and device usage. The method may include training a model to generate a health conclusion based on the collected data. The device usage may comprise an amount of time spent on the device. The data on the device usage may comprise an amount of time spent on one or more apps or categories thereof. The data on the user's positioning may comprise location data taken at multiple points in time. The data on the user's positioning may comprise a local condition at the user's position(s), such as weather, news, local events, or any combination thereof. The data on the voice may comprise first and second instances of the user's voice. The first and second instances may comprise at least one of a vocal cord characteristic, a speech characteristic, and a background noise characteristic.

The disclosure provides a computer implemented method of training a model to generate health information for a user. The method may include collecting data about a user. The method may include training a model to generate health information based on the collected data.

The disclosure provides a computer implemented method of training a model for assessing a mental health status or a change thereof of a user. The method may include collecting marker values of a population of test users, the marker values drawn from at least two of passive data, active data, self-reported data, and external data. The method may include extracting a set of features from the marker values. The method may include training a model using the set of features, wherein the model assesses a mental health status based on the set of features.

The disclosure provides a computer implemented method of training a model for assessing a performance outcome of a user. The method may include collecting marker values of a population of test users, the marker values drawn from at least two of passive data, active data, self-reported data, and external data. The method may include extracting a set of features from the marker values. The method may include training a model using the set of features, wherein the model assesses a mental health status based on the set of features. The performance outcome may include attrition, grades, changes in major, taking longer to graduate, retention, or academic performance.

The disclosure provides a computer implemented method of predicting a performance outcome of a user. The method may include collecting marker values of a population of test users, the marker values drawn from at least two of: passive data, active data, self-reported data, and external data. The method may include extracting a set of features from the marker values. The method may include predicting, using a trained model, a performance outcome of the user based on the set of features.

The disclosure provides a computer implemented method of assessing a mental health status or a change thereof of a user. The method may include collecting marker values of the user from two or more data channels. The method may include extracting a set of features from the marker values. The method may include training a model using the set of features, wherein the model assesses a mental health status based on the set of features. The method may include using the model trained pursuant to the method of claim 1 to assess a mental health status of the user.

The disclosure provides a method of generating a treatment plan to a user. The method may include collecting a set of features from an application on a communication device of a user. The set of features may include voice data, textual data, location data, application usage data, biometric data, sleep data, activity data, self-reported data, and any combination of the foregoing. The method may include processing the set of features, using a neural network, to encode sentiment content from the set of features to determine a marker. The neural network may be configured to process missing features in the set of features. The encoding discards semantic content from the set of features. Markers may be predictive of the user's response to an intervention, which could be, for example, one or more of therapy, a resource or per group, or a drug. The method may include determining an indication of a sentiment of the user based on the encoded sentiment content. The method may include generating a intervention plan to the user based on the user's profile. The profile may include the user's user preferences of the application, the user's demographic information, and/or the user's engagement with the application.

The disclosure provides a method of training a model for generate a treatment plan for a user. The method may comprise collecting a set of features from an application on a communication device of a user, wherein the set of features comprises: voice data: textual data, wherein the textual data comprises text and character depicted expression: location data: application usage data: biometric data; sleep data: activity data; and self-reported data. The method may comprise training a first neural network to encode sentiment content from the set of features to determine a marker, wherein the neural network is configured to process missing features in the set of features, wherein the encoding discards semantic content from the set of features, and wherein the marker is predictive of the user's response to an intervention, wherein the encoded sentiment content provides an indication of a sentiment of the user. The method may comprise training a second neural network to generate a treatment plan to the user based on the user's profile, wherein the profile comprises: the user's user preferences of the application, the user's demographic information, and the user's engagement with the application.

The disclosure provides systems that implement any of the methods of the invention.

The disclosure provides a system for training a model to assess a mental health status of a user. The system may include one or more processors. The system may include a memory including executable instructions which, when executed by the one or more processors. The programming may cause the system to collect marker values of a population of test users from two or more data channels, selected from passive data channels, active data channels, self-reported data channels, and external data channels. The programming may cause the system to extract a set of features from the marker values. The programming may cause the system to train a model using the set of features, wherein the model assesses a mental health status based on the set of features.

The disclosure provides a system for assessing a mental health status or a change thereof of a user. The system may include one or more processors. The system may include a memory including executable instructions. The executable instructions may cause the system to collect marker values of the user from the two or more data channels. The executable instructions may cause the system to use the model trained pursuant to the methods described herein to assess a mental health status of the user.

The disclosure provides a system for improving retention of students or employees. The system may be configured to perform the method on a set of students or employees and thereby improving retention of students or employees of the set. The system may be configured to continuously collect marker values of the population of test users. The system may be configured to continuously collect marker values of the user.

The disclosure provides a system to extract a health conclusion from device usage data. The system may include one or more processors. The system may include a memory including executable instructions. The executable instructions may cause the system to collect a user's device usage data at one or more points in time. The executable instructions may cause the system to use a model to draw a health conclusion based on the device usage data. Device usage data may include total time a user spent on a device. Device usage data may include total time using one or more specific apps. Device usage data may include total time using one or more categories of apps.

The categories may include any one of social, entertainment, educational, and informational.

The disclosure provides a system to extract a health conclusion from a user's device. The system may include one or more processors. The system may include a memory including executable instructions. The executable instructions may cause the system to collect from a device at multiple points in time data on a user's positioning, voice, and device usage. The executable instructions may cause the system to use a model to draw a health conclusion based on the collected data.

The disclosure provides a system to provide health information for a user. The system may include one or more processors. The system may include a memory including executable instructions. The executable instructions may cause the system to collect data about a person. The executable instructions may cause the system to use a model to draw a health conclusion based on the collected data. The executable instructions may cause the system to provide at least one health resource option based on the health conclusion. Data may be self-reported. The self-reported data may be private. The self-reported data may be encoded.

The disclosure provides a system for training a model to generate a health conclusion from location data. The system may include one or more processors. The system may include a memory including executable instructions. The executable instructions may cause the system to collect location data on a user at one or more points in time. The executable instructions may cause the system to collect data on a local condition at the user's position(s). The executable instructions may cause the system to extract marker values from the location data and local-conditions data. The executable instructions may cause the system to train a model to generate a health conclusion based on marker values.

The disclosure provides a system for training a model to generate a health conclusion from a user's voice. The system may include one or more processors. The system may include a memory including executable instructions. The executable instructions may cause the system to collect from a first instance of a user's voice at least one of a vocal cord characteristic, a speech characteristic, and a background noise characteristic. The executable instructions may cause the system to collect from a second instance of a user's voice at least one of a vocal cord characteristic, a speech characteristic, and a background noise characteristic. The executable instructions may cause the system to train a model to generate a health conclusion based on characteristics collected from the first and second recordings.

The disclosure provides a system for training a model to generate a health conclusion from device usage data. The system may include one or more processors. The system may include a memory including executable instructions. The executable instructions may cause the system to collect a user's device usage data at one or more points in time. The executable instructions may cause the system to train a model to generate a health conclusion based on the device usage data.

The disclosure provides a system for training a model to generate a health conclusion from a user's device. The system may include one or more processors. The system may include a memory including executable instructions. The executable instructions may cause the system to collect from a device at multiple points in time data on a user's positioning, voice, and device usage. The executable instructions may cause the system to train a model to generate a health conclusion based on the collected data.

In some embodiments, the data on the device usage comprises an amount of time spent on the device. In some embodiments, the data on the device usage comprises an amount of time spent on one or more apps or categories thereof. In some embodiments, the data on the user's positioning comprises location data taken at multiple points in time. In some embodiments, the data on the user's positioning comprises a local condition at the user's position(s), such as weather, news, local events, or any combination thereof. In some embodiments, the data on the voice comprises voice comprises first and second instances of the user's voice. In some embodiments, the first and second instances comprise at least one of a vocal cord characteristic, a speech characteristic, and a background noise characteristic.

The disclosure provides a system for training a model to generate health information for a user. The system may include one or more processors. The system may include a memory including executable instructions. The executable instructions may cause the system to collect data about a user. The executable instructions may cause the system to train a model to generate health information based on the collected data.

The disclosure provides a system for training a model to assess a mental health status of a user. The system may include one or more processors. The system may include a memory including executable instructions. The executable instructions may cause the system to collect marker values of a population of test users, the marker values drawn from at least two of passive data, active data, self-reported data, and external data. The executable instructions may cause the system to extract a set of features from the marker values. The executable instructions may cause the system to train a model using the set of features, wherein the model assesses a mental health status based on the set of features.

The disclosure provides a system for training a model to assess a performance outcome of a user. The system may include one or more processors. The system may include a memory including executable instructions. The executable instructions may cause the system to collect marker values of a population of test users, the marker values drawn from at least two of passive data, active data, self-reported data, and external data. The executable instructions may cause the system to extract a set of features from the marker values. The executable instructions may cause the system to train a model using the set of features, wherein the model assesses a mental health status based on the set of features. The performance outcome may include attrition, grades, changes in major, taking longer to graduate, retention, or academic performance.

The disclosure provides a system for assessing a performance outcome of a user. The system may include one or more processors. The system may include a memory comprising executable instructions which, when executed by the one or more processors, cause the system to: collect marker values of a user, the marker values drawn from at least two of: passive data, active data, self-reported data, and external data: extract a set of features from the marker values; and predict, using a model, a performance outcome of the user based on the set of features.

The disclosure provides a system for training a model to assess a mental health status of a user. The system may include one or more processors. The system may include a memory including executable instructions. The executable instructions may cause the system to collect marker values of a population of test users. Marker values may be at least two of passive data, active data, self-reported data, and external data. The executable instructions may cause the system to train a model using the set of features, wherein the model assesses a mental health status based on the set of features.

The disclosure provides a system for identifying a health conclusion from location data. The system may include one or more processors. The system may include a memory including executable instructions. The executable instructions may cause the system to collect location data on a user at one or more points in time. The executable instructions may cause the system to collect data on a local condition at the user's position(s). The executable instructions may cause the system to use a model to draw a health conclusion based on the location data and local-conditions data. The local conditions may include weather, news, local events, or any combination thereof. The model may consider multiple local conditions. The model may consider more than one user. The one or more processors may be configured to generate a list of curated resources.

The disclosure provides a system to extract a health conclusion from a user's voice. The system may include one or more processors. The system may include a memory including executable instructions. The executable instructions may cause the system to collect from a first instance of a user's voice at least one of a vocal cord characteristic, a speech characteristic, and a background noise characteristic. The executable instructions may cause the system to collect from a second instance of a user's voice at least one of a vocal cord characteristic, a speech characteristic, and a background noise characteristic. The executable instructions may cause the system to use a model to draw a health conclusion based on characteristics collected from the first and second instances. The voice data may be recorded by the user. The voice data may be streamed by the user.

The disclosure provides a system to assess a mental health status of a user. The system may include one or more processors. The system may include a memory including executable instructions. The executable instructions may cause the system to collect marker values of the user from two or more data channels. The executable instructions may cause the system to extract a set of features from the marker values. The executable instructions may cause the system to train a model using the set of features, wherein the model assesses a mental health status based on the set of features. The executable instructions may cause the system to use the trained model to assess a mental health status of the user.

The disclosure provides a system to generate an intervention plan to a user. The system may include one or more processors. The system may include a memory including executable instructions. The executable instructions may cause the system to collect a set of features from an application on a communication device of a user, where-in the set of features may include voice data, textual data, location data, application usage data, biometric data, sleep data, activity data, self-reported data, or combinations of the foregoing. The executable instructions may cause the system to process the set of features, using a neural network, to encode sentiment content from the set of features to determine a marker, wherein the neural network may be configured to process missing features in the set of features, wherein the encoding discards semantic content from the set of features, and wherein the marker may be predictive of the user's response to an intervention or a therapy. The executable instructions may cause the system to determine an indication of a sentiment of the user based on the encoded sentiment content. The executable instructions may cause the system to generate a treatment plan to the user based on the user's profile. The profile may include the user's user preferences of the application, the user's demographic information, and the user's engagement with the application.

The disclosure provides a system to generate a treatment plan to a user, the system comprising: one or more processors: a memory comprising executable instructions which, when executed by the one or more processors, cause the system to: collect a set of features from an application on a communication device of a user, wherein the set of features comprises: voice data; textual data, wherein the textual data comprises text and character depicted expression: location data: application usage data: biometric data: sleep data: activity data; and self-reported data: train a neural network to encode sentiment content from the set of features to determine a marker, wherein the neural network is configured to process missing features in the set of features, wherein the encoding discards semantic content from the set of features, wherein the marker is predictive of the user's response to an intervention, and wherein the encoded sentiment content is indicative of a sentiment of the user; train a second neural network to generate a treatment plan to the user based on the user's profile, wherein the profile comprises: the user's user preferences of the application, the user's demographic information, and the user's engagement with the application.

Any of the methods described herein may be computer implemented, and the instructions may be provided on a computer-readable medium.

For any of the systems and methods used for assessing a user's mental health, an output may include referring the user to a mental health resource. Mental health resource may be selected based on a model. The model may include a machine learning model. The model may be trained based on data from users of the computer implemented method of assessing a mental health status or a change thereof. The referring may be done via a computing device or system. Mental health resource may be delivered via a computing device or system. Mental health resource may be selected based on a model that may account for one or more of the following data types: mental health status of the user, sexual identity of the user, cultural background of the user, religious beliefs of the user, hobbies and interests of the user, location of the user, and combinations thereof.

For any of the systems and methods used for assessing a user's mental health, an output may include providing a list of curated resources. Any of the methods described herein may include identifying resources that may be curated. Any of the methods described herein may include using of a predefined table and/or dataset that matches resource options to health conclusions. Any of the methods described herein may include selecting the resource option(s) to provide based on a ranking of available options. Any of the methods described herein may include updating the data collection, generating an updated health conclusion, and providing an updated resource option.

For any of the systems and methods used for assessing a user's mental health Assessing a mental health status or a change thereof may include assessing a change in mental health status of the user. Assessing a mental health status or a change thereof may include assessing a baseline mental health status of the user. Assessing a mental health status or a change thereof may include assessing a change in mental health status of the user relative to a baseline mental health status of the user. Assessing a mental health status or a change thereof may include predicting a mental health trajectory of the user. Assessing a mental health status or a change thereof may include calculating a probability of a mental health status of the user.

For any of the systems and methods used for assessing a user's mental health Assessing may include ongoing monitoring of the mental health status of the user. The set of features may include features from two or more of the data channels. The set of features may include features from three or more of the data channels. The set of features may include features from four of the data channels.

For any of the systems and methods used for assessing a user's mental health Device usage data may be encoded. Device usage data may be encoded by extracting sentiment and not semantic content. Device usage data may be encoded by a token to randomize said device usage data. Device usage data may include data derived from one or more screenshots. Data derived from one or more screenshots may include phone usage. One or more screenshots may include application usage on a phone. Data derived from one or more screenshots may include health data from a health tracking application.

For any of the systems and methods, the marker may account for hormonal cycles. For any of the systems and methods, the biometric data may include the changes accounting for hormonal cycles.

For any of the systems and methods, the mental health condition may include for example depression, anxiety, behavior, PTSD, eating disorders, bipolar, schizoaffective disorders, or other conditions, as well as sub-clinical conditions such as loneliness, acceptance, and isolation, and combinations of the foregoing. The behavior may include substance use and/or substance abuse. For any of the systems and methods, training the model may include more than one user. For any of the systems and methods, the model may consider some specific combination of features. For any of the systems and methods, the model may consider changes in device usage data over time. For any of the systems and methods, the health conclusions may include depression, anxiety, behavior. For any of the systems and methods, the one or more processors being configured to cause the system to perform the steps of any prior claim may be configured to generate a list of curated resources. For any of the systems and methods, the one or more processors being configured to cause the system to perform the steps of any prior claim may be configured to identify resources that may be curated. For any of the systems and methods, the one or more processors being configured to cause the system to perform the steps of any prior claim may be configured to use a predefined table and/or dataset that matches resource options to health conclusions. For any of the systems and methods, the one or more processors being configured to cause the system to perform the steps of any prior claim may be configured to select the resource option(s) to provide based on a ranking of available options. For any of the systems and methods, the one or more processors being configured to cause the system to perform the steps of any prior claim may be configured to update the data collection, generating an updated health conclusion, and providing an updated resource option.

For any of the systems and methods, the intervention may include anti-psychotic or mood-altering medication. For any of the systems and methods, the intervention may include counseling. For any of the systems and methods, the intervention may include following a sleep schedule, peer support, immediate exercises, meditation, and other interventions disclosed herein. The concepts addressed herein are not limited to a particular intervention but may include any appropriate intervention, either alone or in any combination. Additional example interventions include peer support, immediate exercises, and meditation.

For any of the systems and methods, the user may be stratified into a group based on the user's historical data, the historical data including history of trauma, adverse childhood experiences, family history, personal history, personal characteristics, or any combination thereof. For any of the systems and methods, the treatment plan may be designed to improve an academic performance (matriculation/retention) of the user.

For any of the systems and methods, the treatment plan may be designed to improve an academic performance (matriculation/retention) of the user.

For any of the systems and methods, the user may be stratified into a group based on the user's historical data, the historical data including history of trauma, adverse childhood experiences, family history, personal history, personal characteristics, or any combination thereof.

For any of the systems and methods, the voice data may be processed by one or more of an artificial neural network (e.g. autoregressive neural network, a recurrent neural network, a LSTM neural network, a large language model, and/or a transformer).

For any of the systems and methods Features may be selected using summary statistics. Features may be selected based on a latent space. The latent space may be based on a transformation of the marker values into the latent space. Features of the set of features selected may improve the assessment of the mental health status. An absence of a marker value may be one of the set of features. Marker values from a passive data channel may include device usage data selected from app usage, battery usage and charging, call frequency and duration, location tracking data, mental health-related internet searches, overall screen time, category specific screen time, physical activity levels (e.g., step counts), sleep patterns inferred from phone activity, social media usage patterns, text message frequency, typing speed and pressure, usage of mental health apps, voice tone and pitch analysis during calls, and frequency and content changes in photos and videos. Category specific screen time may be selected from social, entertainment, educational, and informational.

Marker values from a passive data channel may include wearables data selected from a user's heartrate, body temperature, activity, sleep, respirations, menstrual status, stress level, and combinations thereof. The wearables data may include activity data and may be selected from steps taken, floors climbed, intensity minutes, calories burned, and combinations thereof. The wearables data may include sleep data and may be selected from bedtime, wake up time, sleep duration, quality of sleep, and combinations thereof.

Marker values from a self-reported data channel may include an emotional identifier. Marker values from a self-reported data channel may include a daily emotional identifier.

Marker values from the passive data channel may include location data. The location data may be selected from location, time spent at location, location type, and location frequency. The location may be selected from home, gym, school, restaurant, bar, church, and other.

Marker values may include values from a self-reported data channel. The self-reported data channel may include values from self-reported data. Marker values from a self-reported data channel may include data from a questionnaire. The questionnaire may be completed by a user, a user's supervisor, a user's co-worker, a user's teacher, a user's counselor, a user's family member, a user's friend, or a combination thereof. The questionnaire may be completed online or in a paper format. Marker values may include values from an active data channel. Marker values from an active data channel may include voice values data. Voice values data may be selected from voice characteristics, speech characteristics, background noise characteristics, and combinations thereof. Voice values data may include passive noise data. Voice values data may be selected from tone of voice, inflection of voice, word count, speech rate, intensity of voice, pitch, magnitude, phonetics, tempo-spectral, formant, glottal closure instance and combinations thereof.

Marker values may include values from an external data channel. Values from an external data channel may be selected from weather reports, local current events, and global current events.

A computer implemented method for assessing a mental health status or a change thereof of a user may include collecting marker values of a population of test users. Marker values may be drawn from at least two of passive data, active data, self-reported data, and external data. The method may include extracting a set of features from the marker values for training a model.

The computer implemented method for assessing a mental health status or a change thereof of a user may include collecting location data on a user at one or more points in time. The method may include collecting data on a local condition at the user's position(s). The method may include using a model to draw a health conclusion based on the location data and local-conditions data. The local conditions may include weather, news, local events, or any combination thereof. The model may consider multiple local conditions. The model may consider more than one user.

The method may include continuously collecting marker values of the population of test users. The method may include continuously collecting marker values of the user. The continuous markers may be collected over 3 months. The continuous markers may be collected over 6 months. The continuous markers may be collected over 1 year. The continuous markers may be collected over a semester. The continuous markers may be collected over 2 semesters.

The method may include encoding the marker values from the data channels. The encoding may include randomization of the marker values from the data channels. The encoding may include extracting sentiment content and discarding semantic content from the marker values from the data channels.

The method may include providing a list of curated resources. The list may be provided as a data structure, such as a database.

The method may include reporting a clinical mental health status of the user. Reporting a clinical mental health status of the user may include reporting an anxiety or depression status of the user. Reporting a clinical mental health status of the user may include reporting a subclinical mental health status of the user. Reporting a sub-clinical mental health status of the user may include reporting acceptance or loneliness.

The method may include using the model to predict a matriculation status of the user. The systems and methods may be used to improve retention of students or employees, the method including performing the method of claim 51, on a set of students or employees and thereby improving retention of students or employees of the set.

The model may be trained using a machine learning algorithm selected from one or any combination of principal component analysis (PCA), uniform manifold approximation and projection (UMAP), artificial neural networks (e.g. variational autoencoder (VAE), recurrent neural networks (RNNs), long short-term memory networks (LSTMs), and transformers), time series, penalized regression, and non-penalized regression.

The model may include a machine learning model. The model may be trained based on data from users of the computer implemented system to assess a mental health status.

The population of test users may be students. The user may be a student. The student may be a college student. The population of test users may be substantially 18 to 24 years of age. The population of test users may be at least 80% 18 to 24 years of age. The population of test users may be at least 90% 18 to 24 years of age. The user may be 18 to 24 years of age. The population of test users may be employees. The population of test users may be members of the military. A user may be an employee. A user may be a member of the military.

The questionnaire may include questions related to demographic, family history, health history, impairments, hobbies, mental health history, family mental health history, academic history, romantic history, exercise details, drug and alcohol use and history, sleep, diet, emotional status and history, socialization, recurrent thoughts, physical and biological signs, or a combination thereof. The questionnaire may include demographic questions selected from age, sex, gender identity, sexual orientation, race, ethnicity, religion, or any combination thereof.

The referring may be done via a computing device or system. The mental health resource may be delivered via a computing device or system. The mental health resource may be selected based on a model that may account for one or more of the following data types: mental health status of the user, sexual identity of the user, cultural background of the user, religious beliefs of the user, hobbies and interests of the user, location of the user, and combinations thereof.

The self-reported data may include age, sex, gender identity, sexual orientation, race, ethnicity, religion, or any combination thereof. The self-reported data may include an emotional identifier. Marker values from a self-reported data channel may include a daily emotional identifier.

The system and method may assess use by the user of the mental health resources. The assessing use by the user may include assessing time the user may be at a location of the mental health resource. The assessing use by the user may include assessing time the user interacts with a website of the mental health resource. The assessing use by the user may include assessing data generated from an app used to provide the mental health resource. The assessing use by the user may include assessing changes in the mental health status of the user. The assessing use by the user may include assessing feedback from the user regarding the mental health resource. The system may be configured to assess time the user may be at a location of the mental health resource. The system may be configured to assess time the user interacts with a website of the mental health resource. The system may be configured to assess data generated from an app used to provide the mental health resource. The system may be configured to assess changes in the mental health status of the user. The system may be configured to assess feedback from the user regarding the mental health resource. The system may be configured to report a clinical mental health status of the user. The one or more processors being configured to cause the system to report a clinical mental health status of the user may be configured to report an anxiety or depression status of the user. The system may be configured to report a subclinical mental health status of the user. The system may be configured to report acceptance or loneliness. The system may be configured to use the model to predict a matriculation status of the user.

The system may be configured to assess a change in the mental health status of the user. The system may be configured to assess a baseline mental health status of the user. The system may be configured to assess change in mental health status of the user relative to a baseline mental health status of the user. The system may be configured to predict a mental health trajectory of the user. The system may be configured to calculate a probability of a mental health status of the user. The system may be configured to refer the user to a mental health resource. Mental health resource may be selected based on a model.

The system may be configured to cause the system to continuously monitor the mental health status of the user. The system may be configured to collect application data. The application data may comprise a profile of the user (e.g., a user profile). The application data may comprise a set of features. The application data may comprise a profile of the user and a set of features. The set of features may include features from two or more of the data channels. The set of features may include features from three or more of the data channels. The set of features may include features from four of the data channels.

The system may be configured to encode the marker values from the data channels. The system may be configured to randomize the marker values from the data channels. The system may be configured to extract sentiment content and discard semantic content from the marker values from the data channels.

The system may provide for inputs from third party. The third party may be a user, a user's supervisor, a user's co-worker, a user's teacher, a user's counselor, a user's family member, a user's friend, or a combination thereof. The questionnaire may include questions related to demographic, family history, health history, impairments, hobbies, mental health history, family mental health history, academic history, romantic history, exercise details, drug and alcohol use and history, sleep, diet, emotional status and history, socialization, recurrent thoughts, physical and biological signs, or a combination thereof.

The system may collect continuous markers for at least 3 months. The system may collect continuous markers for at least 6 months. The system may collect continuous markers for at least 1 year. The system may collect continuous markers for at least one semester. The system may collect continuous markers for at least 2 semesters.

Voice values data may be selected from tone of voice, inflection of voice, word count, speech rate, intensity of voice, pitch, magnitude, phonetics, tempo-spectral, formant, glottal closure instance and combinations thereof.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative, and not as restrictive.

### INCORPORATION BY REFERENCE

All publications, patents, and patent applications mentioned in this specification are herein incorporated by reference to the same extent as if each publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The following figures illustrate embodiments of the systems and methods described herein. The drawings are illustrative only and are not intended to limit the scope of the invention:

FIG. 1 depicts an example of a system for assessing a mental health status or a change thereof of a user and providing resources for the user.

FIG. 2 depicts an example of a process for assessing a mental health status or a change thereof of a user and providing resources for the user.

FIGS. 3A-3F depicts an example graphical user interface (GUI) for an application assessing a mental health status or a change thereof of a user and providing resources for the user. FIG. 3A depicts a view of a home screen. FIG. 3B depicts a view of a weekly check-in activity screen. FIG. 3C depicts a view for a user to provide voice data. FIG. 3D depicts a view of a report screen. FIG. 3E depicts a view of a resources screen. FIG. 3F depicts a view of a login screen.

FIG. 4 shows an example of a computing device: in this case, a device with one or more processors, memory, storage, and a network interface.

FIG. 5 shows an example of a web/mobile application provision system; in this case, a system providing browser-based and/or native mobile user interfaces.

FIG. 6 shows an example of a cloud-based web/mobile application provision system; in this case, a system comprising an elastically load balanced, auto-scaling web server and application server resources as well synchronously replicated databases.

FIGS. 7A-7B depicts daily contribution of data overtime for particular biomarkers over two months. FIG. 7A depicts data collection of health and location data. FIG. 7B depicts data collection of app usage, daily check-in, voice, and weekly check-in data.

FIG. 8 depicts changes in generalized anxiety disorder (GAD) 7-item anxiety scale for select users over 8 weeks.

FIG. 9 depicts changes in responses to PHQ-8 (Patient Health Questionnaire-8) for select users over 8 weeks.

FIG. 10 depicts step count stratified by high and low GAD7-item anxiety scale for users over 70 days.

FIG. 11 depicts step count stratified by high and low PHQ-8 for users over 70 days.

FIG. 12 depicts a schematic of a framework for training a machine learning algorithm.

FIGS. 13A-13B depicts an examination of internal consistency of responses by comparing the daily emoji reports with the validated PANAS instrument.

FIGS. 14A-14H depicts a method for computing app usage by a user.

### DETAILED DESCRIPTION OF THE INVENTION

This disclosure presents methods and systems for precision mental health. The methods and systems use markers for early detection and monitoring of mental health conditions. The methods and systems use machine learning techniques to train models for assessing mental health conditions.

### Assessing Mental Health

This disclosure presents methods and systems for using markers for assessing mental health. The assessing of mental health may include assessing a condition or disorder. The assessing of mental health may include assessing a mental health status or a change thereof, assessing a trend in a mental health status, or both. The assessing may include identifying an improvement or decline in a mental health status. The assessing may be conducted at a single time point. The assessing may be conducted over time at a series of timepoints. The assessing may be conducted longitudinally. The assessing may be substantially continuous or substantially real-time.

Examples of mental health conditions that may be assessed using the systems and methods of the invention include all types of clinical and sub-clinical mental, psychological, behavioral, and brain health and related conditions, including:
Mood Disorders, which affect a person's emotional state. Examples include Major Depressive Disorder, Bipolar Disorder, Dysthymia, and Cyclothymic Disorder. Mood Disorders may be assessed using the methods and systems of the disclosure.
Anxiety Disorders, which are characterized by excessive fear or anxiety. This category includes, for example, Generalized Anxiety Disorder, Panic Disorder, various Phobias, Obsessive-Compulsive Disorder, Post-Traumatic Stress Disorder, and Separation Anxiety Disorder. Anxiety Disorders may be assessed using the methods and systems of the disclosure.
Psychotic Disorders, like Schizophrenia, involve distorted thinking and awareness. Psychotic Disorders may be assessed using the methods and systems of the disclosure.
Eating Disorders, which are characterized by abnormal or disturbed eating habits. Common examples include, for example, Anorexia Nervosa, Bulimia Nervosa, and Binge-Eating Disorder. Eating Disorders may be assessed using the methods and systems of the disclosure.
Impulse Control and Addiction Disorders, which include conditions where individuals struggle to resist urges, such as, for example, Alcohol and Substance Use Disorders, Gambling Disorder,
Kleptomania, and Pyromania. Impulse Control and Addiction Disorders may be assessed using the methods and systems of the disclosure.
Personality Disorders, which involve enduring, inflexible, and pervasive patterns of behavior and inner experience. These include, for example, Borderline Personality Disorder, Antisocial Personality Disorder, Narcissistic Personality Disorder, and Avoidant Personality Disorder. Personality Disorders may be assessed using the methods and systems of the disclosure.
Obsessive-Compulsive and Related Disorders, which are typified by a preoccupation with orderliness, perfection, and control. Examples include Obsessive-Compulsive Disorder, Body Dysmorphic Disorder, Hoarding Disorder, Trichotillomania, and Excoriation Disorder. Obsessive-Compulsive and Related Disorders may be assessed using the methods and systems of the disclosure. Trauma- and Stressor-Related Disorders, which are related to the response to traumatic or stressful events, including, for example, Post-Traumatic Stress Disorder, Acute Stress Disorder, and Adjustment Disorders. Trauma- and Stressor-Related Disorders may be assessed using the methods and systems of the disclosure.
Dissociative Disorders, which involve problems with memory, identity, emotion, perception, and behavior, such as, for example, Dissociative Identity Disorder, Dissociative Amnesia, and Depersonalization/Derealization Disorder. Dissociative Disorders may be assessed using the methods and systems of the disclosure.
Sleep-Wake Disorders, which affect the quality, timing, and amount of sleep, causing distress and impairment in daytime functioning. Examples include Insomnia Disorder, Sleep Apnea, and Narcolepsy. Sleep-Wake Disorders may be assessed using the methods and systems of the disclosure.
Neurodevelopmental Disorders, which typically manifest early in development and include, for example, Autism Spectrum Disorder, Attention-Deficit/Hyperactivity Disorder, and Learning Disorders. These conditions generally appear before a child enters grade school and can significantly impact developmental progress. Neurodevelopmental Disorders may be assessed using the methods and systems of the disclosure.
Subclinical conditions that are early signs of mental health changes, for example loneliness, acceptance, and belonging.

### Mental Health Assessment Output

The system may provide a mental health assessment to a user. The mental health assessment may be based on a performance outcome of the user. The system may provide a mental health assessment in the form of a report or notification. The system may provide a mental health assessment to the user on an app. The system may provide a mental health assessment to the user on a smart device. The assessment may be available on the app for the user to access at any time. The assessment may alert the user to changes in mental health status. The assessment may alert the user to a decline in mental health status. The assessment may alert the user to an improvement in mental health status. The assessment may provide trends in changes in mental health.

The assessment may include recommendations for intervention. The recommended interventions can be a treatment plan. Recommendations for intervention may include recommendations for resources related to improving the specific mental health status of the user. Recommendations (e.g., a profile of the user) may be tailored to specific characteristics of the user. Examples of characteristics of the user include preferences of the user, demographics of the user, and engagement of the user. As an example, recommendations may provide resources related to the user's mental health status and one or more other characteristics, such as sexual identity or religious preference.

A profile of a user may comprise the application preferences of the user. A profile of a user may comprise demographic information of the user. A profile of a user may comprise the engagement of the user with application. A profile of a user may comprise the application preferences of the user, demographic information of the user, engagement of the user with the application, or any combination thereof.

The systems and methods may make use of a computer comprising a distributed computing network. The methods may include a computer providing as an output a mental health assessment that has been produced by the systems and methods using a distributed computing network. The methods may include a user collecting a mental health assessment that has been produced by the systems and methods using a distributed computing network.

### Mental Health Assessment of Populations

The systems and methods may monitor mental health of a population. For example, the population may be a set of students or employees. The assessment may be provided to an employer or individual responsible for managing or overseeing the population's mental health. The assessment may be provided on a smart device. The assessment may include alerts about changes in mental health status of the population or a portion of the population. The assessment may provide alerts about declines in mental health status of the population or a portion of the population. The assessment may provide alerts about an improvement in mental health status of the population or a portion of the population. The assessment may be provided in a manner that protects the privacy of individuals, e.g., by excluding individually identifiable information.

The assessment may include recommendations for intervention to improve the mental health status of the population or a subset of the population. Examples of subsets of the population may include factory floor workers of a company, pilots of an airline, police officers of a law enforcement agency, a specific sports team at a school, a minority population of a school or business, a disadvantaged subpopulation, or a subpopulation facing discrimination or systemic disadvantages.

Examples of interventions suitable for a company may include employee assistance programs, flexible work arrangements, wellness programs, mental health days, stress management workshops, training for managers, open communication channels, work-life balance initiatives, mental health awareness campaigns, support groups or peer networks, a healthy workplace environment, professional development opportunities, financial wellness programs, regular check-ins, and crisis intervention resources.

Examples of interventions suitable for a university may include counseling and psychological services, peer support programs, stress management and mindfulness workshops, mental health awareness events, flexible academic accommodations, wellness and fitness programs, on-campus mental health resources, relaxation and quiet zones, student-led support groups, clubhouses, religious groups, academic advising and mentorship, financial aid and scholarship support, diversity and inclusion initiatives, social and recreational activities, online mental health resources and apps, and emergency support services.

Examples of interventions suitable for a high school may include guidance counseling services, peer mentoring programs, stress management workshops, mental health awareness and education sessions, flexible academic accommodations, extracurricular clubs and activities, on-campus wellness programs, student support groups, academic tutoring and support, financial assistance programs, diversity and inclusion initiatives, sports and physical fitness activities, art and creative outlets, technology and internet access support, and emergency counseling services.

Examples of interventions suitable for improving mental health of minority students of a university or high school may include cultural sensitivity training for staff and faculty, mentorship programs with minority alumni, support groups for minority students, scholarships and financial aid specifically for minority students, diversity and inclusion workshops and events, safe spaces for cultural expression, language support services, access to minority-focused mental health professionals, career counseling with a focus on diversity, networking events with diverse professionals, educational programs on cultural competence, social justice and advocacy groups, partnerships with minority organizations, multicultural centers and resources on campus, and policies to address discrimination and promote equality.

Interventions may include the implementation of policies relating to any one or more of the foregoing interventions. The policies may include policy statements or requirements about implementation of one or more of the foregoing interventions.

The systems and methods may make use of a computer that comprises a distributed computing network. The methods may include a computer providing as an output an interventions report that has been produced by the systems and methods using a distributed computing network. The methods may include an employer or individual responsible for managing or overseeing the population's mental health collecting an interventions report that has been produced by the systems and methods using a distributed computing network.

### Markers and Marker Values

The methods and systems use markers for early detection and monitoring of mental health conditions. Examples of suitable markers include markers that are collected through:
passive data channels,
active data channels,
self-reported data channels, and
external data channels.

Marker values may be collected from a target population and used in a machine learning technique to train a model for assessing a mental health status or a change thereof. Markers may be obtained from a user and used in a trained model to assess a mental health status of the user.

### Passive Data Channels

The system and methods make use of data from one or more passive data channels. Examples of passive data include location data, wearable data, and device usage data.

The system and methods may be used to extract features from passive data. The system and methods may be used to analyze features extracted from passive data. The system and methods may be used to derive one or more marker values from passive data.

Examples of passive data include those in Table 1.

TABLE 1
Examples of passive data

| | |
|---|---|
| Health | Bedtime |
| | Wake up time |
| | Sleep duration |
| | Heart rate variability |
| | Number of steps/day |
| | Duration being active/day |
| | Duration being sedentary/day |
| | Calories burned/day |
| GPS | Location home, gym, restaurant, bar, church, other |
| | Time spent at each location |
| | Location indoor or outdoor |
| | Location variance |
| | Location entropy |
| | Moving speed |
| | Number of frequent locations visited |
| | Radius and distance traveled |
| App usage | Total screen time per week |
| | Total screen time per day |
| | Screen time per day by category (e.g., social, |
| | entertainment, information, etc.) |

Passive data may include data relating to the location of a user. Location data may, for example, include time spent at a location. Location data may include the type of location. Examples of types of locations include home, gym, school, restaurant, bar, church, and others. Location data may include the frequency and/or duration of visiting one or more locations. Location data may be automatically collected from a user's smart device (e.g., smartphone). Location data may be continuously collected from a user's smart device. Location data may be intermittently collected from the smart device of a user.

Location data may be collected as coordinates. The coordinates may, for example, identify an area such as a residential area. The coordinates may identify a specific location. Location data may, for example, include addresses or other identification of places frequented by a user. For example, places frequented by a college student may include lecture halls, university library, campus cafeteria, dormitory, student union, study lounges, computer labs, campus bookstore, fitness center, sports facilities, local coffee shops, nearby parks, student clubs and organization offices, university health center, off-campus bars, and restaurants. The coordinates may be used to calculate the total distance traveled over a period of time.

As an example, a user using the app has allowed the app to collect and analyze location data based on the location of their smart device. The app collects location data. Consider the following scenarios:
The app identifies a three-day holiday weekend during which the user never leaves the address they entered as their home address. This prompts the app to send a questionnaire to the user mid-week to inquire how many places they visited during the three-day weekend.
The app identifies that the user frequents only two locations: their home and a local bar.
The app identifies that the user is frequently found at locations other than their home between midnight and four a.m.

In each of these examples, the methods may use the location data to generate a mental health status of a user.

As another example, a user using the app is identified as frequenting a bar three times a week and a church once a week. The user's location data indicates a gradual change in their location data over one year. The user is now frequenting a bar five times a week and no longer attending church. The location data may be used to generate a user's mental health trajectory.

Passive data may include wearables data. Wearables data may include data collected from a smartphone. Wearables data from a smartphone may be a part of an app separate from the app. Wearables data may include data collected from a device separate from a smartphone.

Examples of wearables include smartwatches, smart rings, pedometers, activity/fitness trackers, smart clothes, and any wearable computers. Data from wearables may, for example, include heart rate, oxygen levels, body temperature, activity, sleep, respirations, menstrual status, user stress level, active energy, blood glucose, blood oxygen, body fat percentage, body mass index, calories consumed, diastolic blood pressure, exercise minutes, heart rate, height, high heart rate notifications, hydration, irregular rhythm notifications, low heart rate notifications, menstrual cycle tracking, mindful minutes, respiratory rate, sleep, steps, systolic blood pressure, walking and/or running distance, water, weight, workouts, calories burned, or any combination thereof.

Wearables data may include activity data. Activity data from a wearable of a user may include steps taken. Activity data from a wearable of a user may include floors climbed. Activity data from a wearable of a user may include intensity minutes. Activity data from a wearable of a user may include calories burned.

Wearables data may include sleep data. Sleep data from a wearable device may include the user's bedtime. Sleep data from a wearable device may include the waketime of the user. Sleep data from a wearable device may include the sleep duration of the user. Sleep data from a wearable device may include the user's sleep quality.

Passive data may include device usage data. Device usage data may include data collected from a smart device (e.g., a smartphone or a tablet) of a user. Examples of passive data collection include app (e.g., the app and apps other than those disclosed herein) usage, battery usage and charging, call frequency and duration, text frequency, call and text diversity, phone locks and unlocks, phone pickups, location tracking data, screentime, category-specific screentime, physical activity levels (e.g., step counts or move minutes), sleep patterns inferred from phone activity, social media usage patterns, typing speed and pressure, voice tone and pitch analysis during calls, and frequency and content changes in photos and videos. Note that device usage data may be active or passive data. For example, device usage data that requires no user intervention to collect may be passive whereas device usage data that requires user intervention to collect may be active.

App usage may include time spent on user apps. In some embodiments, app usage may include time spent on apps of specific categories. Examples of app categories include social, entertainment, educational, and informational.

Phone call data may include call frequency data. Call frequency data may include a count of calls over a specified time period. Phone call data may include call duration data. Phone call data may call frequency data and/or call duration data relating to outgoing calls, incoming calls, calls to specific numbers, calls from specific numbers, and combinations of the foregoing.

Passive data may include screen time data. Screen time data may include time using a device. Screen time data may combine screen time for multiple devices.

Passive data may include text message data. Text message data may, for example, include number of text messages, number of text messages to unique numbers, text messages sent versus texts collected, grouping of texts (e.g., did a significant number of text messages from timeframe on the users device start with a message that was sent or collected by the user), time of texts, words used in texts. Tracked words may be words associated with mental health, substance use, or emotions. Words may include pictorial representations (e.g., emoticons or emojis) related to mental health, substance use, or emotions.

Text message data may be converted to sentiment data to protect privacy. The app may record sentiments derived from the text data rather than the text words themselves. As an example, a user sends a text message stating “Today was awful. I think I failed my exam. All I want to do is sleep.” The sentiment data may include words or indicators relating to “depression,” “sadness,” and “poor academic performance.” In this example, the phrase “today was awful” was converted to “sad”: “failed exam” was converted to “poor academic performance”; and “all I want to do is sleep” was converted to “depression.” The semantic content of the text message is not retained, but the sentiment of the text message is retained by categorizing the text with keywords.

The passive data may, for example, be collected from a user's smart device. The passive data may be gathered directly by the app. In some embodiments, a user may take images of the screens comprising the data and upload them to the app for image analysis.

### Active Data

The system and methods make use of data from one or more active data channels.

Examples of active data may include voice data, text data, device usage data, and self-reported data.

Active data may include voice data. Voice data may include data such as, for example, the tone of voice, inflection of voice, word count, speech rate, intensity of voice, pitch, magnitude, phonetics, tempo-spectral, formant, glottal closure instances, and time spent between reviewing the prompt and beginning to speak of the user. Additional examples of voice data are included in Table 2.

The system and methods may be used to extract features from active data. The system and methods may be used to analyze features extracted from active data. The system and methods may be used to derive one or more marker values from active data.

To illustrate, the system may prompt a user to record or stream a response to a prompt or question. In some cases, the prompt is a neutral prompt that requests a response that is not anticipated to generate strong emotion.

Examples of prompts include “describing a person the user admires,” “describing a scene from a movie that the user thinks about a lot,” “What is a good piece of advice the use has collected.”

The system may extract features from the voice data. The extracting may be accomplished in real-time as the recording is being made or thereafter.

The system may prompt the user for voice data upon during a first use of the app. The first use voice data may serve as a baseline sample of voice data. The system may prompt the user for voice data each time the user accesses the app. The system may prompt the user for voice data periodically, such as daily or weekly. The system may request periodic voice data about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more times a month. The system may request periodic voice data about 1, 2, 3, 4, 5, 6, 7, 8, 9, 10, 11, 12, 13, 14, 15, 16, 17, 18, 19, 20, or more times a week. The system may request periodic voice data about 1, 2, 3, or more times a day. The system may request periodic voice data about once a day. The system may request periodic voice data about once a week. The system may request periodic voice data about twice a week. The system may request periodic voice data about three times a week. The system may request periodic voice data about four times a week.

The frequency of requests for voice data may increase or decrease based on the user's mental health status as assessed by the systems and methods. The frequency of requests for voice data may increase or decrease based on the user's mental health trajectory as assessed by the systems and methods. The frequency of requests for voice data may increase or decrease based on the user's previous voice response. The frequency of requests for voice data may increase or decrease based on one or more other marker values. For example, a voice response may be added based on location data or other data acquired by the systems and methods

TABLE 2
Examples of data and markers collected from voice.

| | |
|---|---|
| Vocal Markers | Glottal closure instance (GCI) |
| (how sound is | Opening phase (OP) |
| articulated at | Closing phase (CP) |
| vocal cords) | Closed phase (C) |
| | Tempo-spectral-acoustic features. Time or length of the |
| | interval that participants continue an utterance and tempo |
| | Formant features (phonetics) |
| | Mean pitch |
| | Variance of pitch |
| | Mean magnitude |
| | Variance of magnitude |
| | Zero crossing rate (ZCR) (intensity of voice) |
| | portions (how frequently SCR appeared) |
| | Length of recording |
| | Time between recordings |
| | Prompt that was used |

In some embodiments, the active data comprises text data of the user. Text data may include data such as, for example, the words of the text, word count, typing rate, intensity of keyboard taps, amount of erasure or restarting their response, and time spent between reviewing the prompt and beginning to type. The system may prompt the user to input text data in the same manner and timing as described for voice data above.

### Self-Reported Data

The methods make use of data from one or more self-reported data channels. Self-reported data may include responses to prompts or questions. The system may prompt the user to input the data. The prompt may occur, for example, during a user- or system-initiated check-in.

The system and methods may be used to extract features from self-reported data. The system and methods may be used to analyze features extracted from self-reported data. The system and methods may be used to derive one or more marker values from self-reported data.

In some cases, the system may prompt one or more third parties with questions. For example, the third party may be selected from workplace contacts, educational professionals, healthcare providers, support and guidance figures, specialized service providers, and other relevant contacts. Workplace contacts may include supervisors, coworkers, employees, and human resources representatives. Educational professionals may include teachers, professors, school counselors, academic advisors, and school nurses. Healthcare providers may include counselors, therapists, physicians, psychiatrists, psychologists, occupational therapists, nurses, and pharmacists. Support and guidance figures may include family members, friends, coaches, mentors, peer mentors, spiritual leaders, life coaches, and support group members. Specialized service providers may include social workers, case managers, and community health workers. Other relevant contacts may include residence advisors, club or organization leaders, and legal guardians.

The prompts or questions may relate to the user's demographics, family history, health history, impairments, hobbies, mental health history, family mental health history, academic history, romantic history, activities, drug and alcohol use, sleep patterns, diet, emotional status, social engagement, recurrent thoughts, biological and physical characteristics, and adverse and/or significant life events.

Demographic prompts or questions may, for example, relate to the user's age, cultural background, disability status, education level, ethnicity, family structure, gender identity, geographic location, language spoken, marital status, nationality, occupation, race, religion, sex, socio-economic status, sexual orientation, veteran status.

The system may prompt the user or a third party (see the list above) to respond to prompts or questions to generate a baseline of self-reported data on the user. Table 3 lists examples of questions that may be used to gather baseline data.

Baseline information may in some cases be editable by the user. For example, a user may have originally identified their baseline sexual orientation in the app as bisexual but later identified their sexual orientation as gay. The user would update their profile on the app by changing their sexual orientation from bisexual to gay.

The system may prompt the user or third party to answer or update questions periodically. For example, daily, weekly, or monthly. Table 4 lists examples of questions that may be asked weekly.

Questions may have multiple choice answers. For example, a question may ask a user “How many times have you cried this week if at all?” with multiple choice answers such as “(a) 0, (b) 1-2, (c) 3-4, (d) 5 or more”. The user may select an option from among the choices.

A question may provide multiple choice answers and an input field for the user to respond with an answer. As an example, a question may ask the user “What has been on your mind most today?” with choices such as “family, friends, school, sports, work, politics” plus a text field for the user to provide alternative answers when the options provided do not match the frequent thoughts of the user. In this example, the user may choose to fill in that they have been thinking about money or finances.

The system may prompt the user to answer a question. The answer may be a single word, a sentence, a pictorial (e.g., emoticon or emoji). For example, the system may prompt the user daily or multiple times a day to respond to questions such as “How do you feel?” or “What emotion would best describe you in this moment?”.

TABLE 3
Examples of baseline questionnaire questions

| | |
|---|---|
| Demographics | Age |
| | Gender Identity |
| | Sexual Orientation |
| | Race |
| Impairments | Hearing, visual, mobile impairments |
| | Neurodiverse |
| | Learning disability |
| | Chronic health condition |
| Family situation | Adopted |
| | Foreign national or first-generation immigrant |
| | First generation to attend college |
| | Participate in college sport |
| | Identify as a gamer |
| Family history of mental health | Mother, father, sibling, or other relative |
| condition | |
| Personal history | Sought professional help for a MH condition |
| | Diagnosis of mental health condition |
| | GPA and period type |
| | Major |
| | Use of mental health or academic counseling resources |
| | Steady romantic partner |
| Adverse Childhood Experiences | ACES Score. An ACE score is a tally of different types of |
| (ACES) | abuse, neglect, and other childhood experiences that have |
| | been shown to be associated with development of mental |
| | health conditions. |

TABLE 4
Examples of weekly questions/topics

| | |
|---|---|
| Exercise | # days/week exercised |
| | # hours exercise per week |
| | # of types of exercises |
| Meals | Diet plan (e.g., paleo, vegetarian, etc.) |
| | # meals per day |
| | # snacks per day |
| | # meals and snacks per week |
| | # of skipped meals |
| | Frequency of home cooked meals |
| Sleep | # hours/night sleep |
| | # naps/week |
| | Quality of sleep |
| Substance use | # days/week smoking/vaping |
| | # days/week using marijuana |
| | # days/week drinking alcohol |
| | # days/week using a controlled substance |
| | Blackout or hospitalization |
| Socialization | Meals shared |
| | Did you have fun this week? |
| | Did you argue this week? |
| | Have you felt discriminated against? |
| | How accepted do you feel by your social group? |
| | How often have you felt lonely? |
| Top of Mind | Category that is top of mind (e.g., financial, family, |
| | friends, etc.) |
| Physical/Biological symptoms | Have you seen a healthcare provider in the past week? |
| | Have you experienced headaches or stomach aches? |
| | Did you cry because you were sad this week? |
| | Menstrual cycle |
| | Hormonal birth control |
| | Antipsychotic use |

### External Data

The methods make use of data from one or more external data channels. External data can be sourced from any database that can be accessed. Examples of external data include news, crime databases, a user's searches queries on a search engine, a health application for communicating between a user and a doctor, and weather reports. Table 5 provides further examples of external data.

The system and methods may be used to extract features from self-reported data. The system and methods may be used to analyze features extracted from self-reported data. The system and methods may be used to derive one or more marker values from self-reported data.

TABLE 5
Examples of external data

| | |
|---|---|
| Weather | Temperature |
| | Precipitation |
| | Humidity |
| | Hours of light |
| | Cloud coverage |
| | Severe weather (e.g., hurricane, tornado, heat wave) |
| Local Events | Mass shooting |
| | Police brutality |
| | Severe weather (e.g., hurricane, tornado, heat wave) |
| | Local layoffs |
| Global Events | Wars |
| | Severe weather (e.g., hurricane, tornado, heat wave) |

In some embodiments, the external data may include a news report. News reports may include local news. News reports may include national news. News reports may include news reports from a world news.

News reports may relate to the user. For example, if a user is identified as an immigrant, then national news about anti-immigrant sentiments may be used as a part of assessing the user's mental health status.

As another example, a series of armed robberies have taken over the course of a month in a 500-block radius of a business the user frequents. The external data from the news reports in this example may be used to assess the user's increasing anxiety.

External data may include weather reports. A weather report may include local weather, regional weather, national weather, and/or global weather. The weather report may relate to the user. A local weather report may include information about a blizzard. In this example the user remaining home for several days in a row may be appropriately countered by the local weather of the user. A local weather report may include information about a cloudy week in January. The external data from the local weather report in this example may be used to assess the user's increasing depression.

The systems may be configured to collect data longitudinally. The systems may be configured to collect data at various intervals. The data may be collected from a user. The data may be collected about a user from a user's smart device. The data may be collected from an external source (e.g., news outlet). The intervals by which the systems are configured to collect data may change in frequency. The intervals may change based on a user. The intervals may change to be more frequent. The intervals may change to be less frequent. The intervals by which the systems are configured to collect data may include different intervals for different data or data channels. In some embodiments, a single data type may be continuously collected. In some embodiments, a single data type may be collected at least once a minute, at least twice a minute, at least three times a minute, at least four times a minute, at least five times a minute, at least six times a minute, at least seven times a minute, at least eight times a minute, at least nine times a minute, or at least ten times a minute. In some embodiments, a single data type may be collected at least once an hour, at least twice an hour, at least three times an hour, at least four times an hour, at least five times an hour, at least six times an hour, at least seven times an hour, at least eight times an hour, at least nine times an hour, at least ten times an hour, at least twelve times an hour, at least fifteen times an hour, at least twenty times an hour, or at least thirty times an hour. In some embodiments, a single data type may be collected at least once a day, at least twice a day, at least three times a day, at least four times a day, at least five times a day, at least six times a day, at least seven times a day, at least eight times a day, at least nine times a day, at least ten times a day, at least twelve times a day, at least fifteen times a day, at least twenty times a day, at least thirty times a day, at least forty times a day, at least fifty times a day, at least sixty times a day, at least seventy times a day, at least eighty times a day, at least ninety times a day, at least one hundred times a day, at least two hundred times a day, at least three hundred times a day, at least four hundred times a day, or at least five hundred times a day. In some embodiments, a single data type may be collected at least once a week, at least twice a week, at least three times a week, at least four times a week, at least five times a week, at least six times a week, at least seven times a week, at least eight times a week, at least nine times a week, at least ten times a week, at least twelve times a week, at least fifteen times a week, at least twenty times a week, at least thirty times a week, at least forty times a week, at least fifty times a week, at least sixty times a week, at least seventy times a week, at least eighty times a week, at least ninety times a week, at least one hundred times a week, at least two hundred times a week, at least three hundred times a week, at least four hundred times a week, at least five hundred times a week, at least six hundred times a week, at least seven hundred times a week, at least eight hundred times a week, at least nine hundred times a week, at least one thousand times a week, at least two thousand times a week, at least five thousand times a week, at least ten thousand time a week, at least fifteen thousand time a week, at least twenty thousand time a week, at least thirty thousand times a week, at least forty thousand times a week, at least fifty thousand times a week, at least sixty thousand times a week, at least seventy thousand times a week, at least eighty thousand times a week, at least ninety thousand times a week, or at least one hundred-thousand times a week. In some embodiments, a single data type may be collected at least once a year, at least twice a year, at least three times a year, at least four times a year, at least five times a year, at least six times a year, at least seven times a year, at least eight times a year, at least nine times a year, at least ten times a year, at least eleven times a year, at least twelve times a year, at least eighteen times a year, at least twenty-four times a year, at least thirty times a year, at least thirty-six times a year, at least forty-two times a year, at least forty-eight times a year, at least sixty times a year, at least seventy times a year, at least eighty times a year, at least ninety times a year, at least one hundred times a year, at least two hundred times a year, at least three hundred times a year, at least four hundred times a year, at least five hundred times a year, at least six hundred times a year, at least seven hundred times a year, at least eight hundred times a year, at least nine hundred times a year, at least one thousand times a year, at least two thousand times a year, at least five thousand times a year, at least ten thousand time a year, at least fifteen thousand time a year, at least twenty thousand time a year, at least thirty thousand times a year, at least forty thousand times a year, at least fifty thousand times a year, at least sixty thousand times a year, at least seventy thousand times a year, at least eighty thousand times a year, at least ninety thousand times a year, at least one hundred-thousand times a year, at least two hundred-thousand times a year, at least three hundred-thousand times a year, at least four hundred-thousand times a year, at least five hundred-thousand times a year, at least six hundred-thousand times a year, at least seven hundred-thousand times a year, at least eight hundred-thousand times a year, at least nine hundred-thousand times a year, at least one million times a year.

In some embodiments, an absence of data is treated as data. For example, a user using the app is regularly responding to requests for data (e.g., voice, text, and self-reporting check-ins). Over time the user begins responding less to these requests. The lack of response from the user will be treated as data along with other data that is collected such as, for example, the time the user does respond to the requests along with the passive data and external data. In some embodiments, a single data may be collected zero times.

The systems may combine the data in any number of ways to assess a user's mental health status or mental health trajectory. The systems may use the data with original variables to assess a user's mental health status or mental health trajectory. The systems may use features extracted from the data to assess a user's mental health status or mental health trajectory. The systems may use the data combined with other data to generate a new data value. The systems may use the features of data combined with features of other data for generating a new data value. Although different characteristics of data have been broken out into user subgroups, the data may be regrouped at any time under a different or new subgroup.

The system may use the data to train one or more machine learning algorithms. The system may extract features of the data to train one or more machine learning algorithms. A machine learning algorithm may be trained on at least tens, at least hundreds, at least thousands, at least tens of thousands, at least hundreds of thousands, at least millions of data points. The system may train a machine learning algorithm to generate early detection flags for a wide variety of clinical and/or sub-clinical mental illnesses. The system may train one or more machine learning algorithms to assess a mental health status and/or trajectory of a user. The system may train one or more machine learning algorithms to determine a mental health status and/or trajectory of a user. The system may analyze the data with a machine learning model. The system may extract features from the data. The system may extract features from the data using a machine learning model. The system may analyze the features with a machine learning model. A system may use a machine learning model to determine a mental health status and/or trajectory of a user. A system may use a machine learning model to determine a retention likelihood of the user. The retention likelihood of the user may include employee retention at a workplace. The retention likelihood of the user may include student retention at a school. The school may include a middle school. The school may include a high school. The school may include a college. The school may include a university. A system may use a machine learning model to predict academic performance of a user. In some embodiments, academic performance comprises grade, time to graduate, matriculation rate, change in degree type (e.g., Bachelor of Art or Bachelor of Science), change in degree level (e.g., associate degree or bachelor's degree), change in major, or any combination thereof.

The system may establish a baseline for a user. The system may assess a mental health status and/or trajectory based on deviations from the baseline of the user. The system may assess a mental health status and/or trajectory by assessing a change in a mental health status of a user. The system may assess a mental health status and/or trajectory by predicting a mental health trajectory of a user. The system may assess a mental health status and/or trajectory by calculating a probability of a mental health status of a user. The system may assess a mental health status and/or trajectory by assessing a deviation from a broader distribution. In some embodiments, the broader distribution includes a distribution of data as associated with a group of people with varying mental health statuses. In some embodiments, changes in mental health can be assessed based on both the user's data (which may be limited, e.g., when they start using the product), and the vast amount of data a model is trained on (which can include data from individuals that have similar history, diagnosis, demographics, and current data patters to the user).

In some embodiments, the methods and systems may include the use of statistics. The statistics may be descriptive. The system may employ statistical tests without correction for multiple hypothesis testing to explore associations between continuous variables and outcomes. The system may use a Welch's two-sample t-test to compare means of prognostic markers between clinical and sub-clinical mental health categories. Examples of clinical mental health categories include depression and anxiety. Examples of sub-clinical mental health categories include loneliness and acceptance. The system may use a chi-square test to compare the distribution of categories between dichotomous clinical outcomes. The system may employ a machine learning model for prediction of clinical outcomes. The machine learning model may include social and behavioral markers.

The system may employ a machine learning model to extract features from data. For example, the system may extract features from vocal data, location data, and/or device usage data. In some embodiments, the app is configured to collect app usage data. In some embodiments, the system accurately parses the app usage data to derive one or more features.

Disclosed herein are methods and systems for assessment of a mental health status. A mental health status may include clinical conditions. Examples of clinical conditions include depression and anxiety. A mental health status may include sub-clinical conditions. Examples of sub-clinical conditions include loneliness and acceptance. Depression can be categorized as minimal, mild, moderate, or severe. Anxiety can be categorized as minimal, mild, moderate, or severe. Loneliness may be assessed on a 1-5 scale. Loneliness may be categorized as low or high. Acceptance may be assessed on a 1-5 scale. Acceptance may be categorized as low or high.

The system may provide one or more resources to a user. The user may collect one or more resources from the systems as disclosed herein. The systems may assess the mental health status of a user. The system may curate the resources for a user. The system may curate the resources for a user based on the mental health status and/or trajectory of the user. The system may curate the resources for a user based on one or more demographics of the user. The system may curate the resources for a user based on one or more preferences identified by the user. The system may curate the resources for a user based on the interactions of the user with the app. The system may curate the resources for a user based on the school of the user. The system may curate the resources for a user based on access of the user. The app may follow up with the user following providing the resources.

The systems may be configured to encode data. The systems may be configured to encrypt data. The systems may be configured to retain a feature and/or value from data and discard the remaining information from the data. The system may be configured to encode sentiment data and/or sentiment content. The system may provide one or more encoding of sentiment content. The system may generate an encoding of sentiment content. The encoding may have fewer dimensions than the data. The encoding may have more dimensions than the data. The encoding may have the same number of dimensions as the data. The encoding may be the conversion of data into a numerical format. The numerical format may be a vector. The encoding may be a one-hot encoding. The encoding may be a binary encoding. The encoding may be the output of a model. The encoding may be the output of a processing layer of the model (e.g., not the final out put of the model). An example of an encoding may include a neural network taking as input a set of features from an application, the model may be configured to output the data after passing the data through the hidden layers of the neural network. In this example, the hidden layers may produce an activation, the activation being the same dimension as the hidden layer, this activation can be thought of as an encoding of the data. The systems may be configured to parse data. The system may retain the parsed data and/or data values and discard additional information from the data.

The systems may include methods to train a model. The model may be a machine learning model. The system may train a model using the data as disclosed herein. The system may train a model using the features as disclosed herein. The system may train a model in an unsupervised manner. The system may train a model in a supervised manner. The system may train a model using reinforcement learning. The system may train a model using transfer learning. The system may train a model using model distillation. The model may have a single output. The model may have two outputs. The model may have multiple outputs. The model may use its own output as input to itself. The model may use the output of another model as input. The model may use a transformation of features as an input.

The model may be a classifier. The system may build a classifier based on the model. The system may build a regression model based on the model. The model may be a regression model. The model may include multiple models. The system may assess a mental health status and/or trajectory using a model. The system may assess a mental health status and/or trajectory using a model developed using the techniques described herein. The system may assess a mental health status and/or trajectory using a model. A mental health condition may be one or more of a healthy condition, a depressed condition, an anxious condition, a loneliness condition, an acceptance condition, or any combination thereof.

The classifier may be a method conducted by a computer system. The method may involve using data and/or features as described herein to output an assessment of a mental health status and/or trajectory. The method may use a classifier. The classifier may take data and/or features as described herein as input. The classifier may output a mental health status and/or trajectory. The classifier may be comprised of multiple steps such as, but not limited to, feature selection, feature transformation, latent space mapping, feature vector composition, feature weighting, input weighting, input into a model, output from a model, analysis of informative features, incorporation of pretrained models, transfer learning, fine-tuning of pretrained models, knowledge distillation, post-processing of model output. The model output may be postprocessed. The classifier may be an artificial neural network, a support vector machine, a linear model, a non-linear model, a parametric model, a non-parametric model, a Bayesian model, a gaussian process, a binary classifier, a multilabel classifier, a non-binary classifier, a deep neural network, an ensemble method, a tree-based model, or a combination thereof. The model may be trained using a dataset composed of data and/or features as described herein.

The model performance may be assessed using metrics such as, but not limited to, receiver operating curve area under the curve (ROCAUC), sensitivity-specificity curve, sensitivity-specificity area under the curve, precision-recall curve, precision-recall area under the curve, precision, recall, sensitivity, specificity, accuracy, f-measure, f1-measure, f2 measure or some combination thereof.

The performance of the model may be determined using at least one output of the model. The performance of the model may be determined using some or all of the internal state of the model. The performance of the model may be greater than about 20%. The performance of the model may be greater than about 30%. The performance of the model may be greater than about 40%. The performance of the model may be greater than about 50%. The performance of the model may be greater than about 60%. The performance of the model may be greater than about 70%. The performance of the model may be greater than about 75%. The performance of the model may be greater than about 77%. The performance of the model may be greater than about 79%. The performance of the model may be greater than about 80%. The performance of the model may be greater than about 82%. The performance of the model may be greater than about 84%. The performance of the model may be greater than about 86%. The performance of the model may be greater than about 88%. The performance of the model may be greater than about 90%. The performance of the model may be greater than about 91%. The performance of the model may be greater than about 92%. The performance of the model may be greater than about 93%. The performance of the model may be greater than about 94%. The performance of the model may be greater than about 95%. The performance of the model may be greater than about 96%. The performance of the model may be greater than about 97%. The performance of the model may be greater than about 98%. The performance of the model may be greater than about 99%. The classifier may be configured in a way to improve computational efficiency measure by, but not limited to, computational complexity, memory use, storage capacity, computational time, power requirements, storage and use on a smart phone, storage and use on a personal computer, storage and use on a cloud-based system, storage and use on a high performance computer system, storage and use from a flash drive.

### Combinations of Data

The system may derive a feature from data combined from two or more data. The system may derive a feature from data combined from two or more data channels. The system may derive a feature using transformations of the features themselves. The system may make transformations through algorithms intended to combine or transform features in a predetermined manner. The system may make transformations by machine learning models in a manner learned during the training of the model. The transformations may use predetermined methods to combine parameters of a machine learning model or derivations of parameters to produce derived features.

The system may use derived features as input to a model. The final output of a model may include derived features. Derived features may be input to another model. Feature selection may use derived features. Derived features may guide another model's behavior.

### Users

The methods and systems are intended to be used by a user. The user may be human. The user may be healthy. The user may have a mental health condition. The user may have a history of a mental health condition. The user may have a family history of mental health conditions. The user may be a student. The user may be a high school student. The user may be a college student. The user may be a university student. The user may be a trade student. The user may be pursuing an associate degree. The user may be pursuing a bachelor's degree. The user may be pursuing a master's degree. The user may be pursuing a doctoral degree. The student may be pursuing certification. The student may be pursuing a trade. The student may be pursuing a veterinary degree. The student may be pursuing a medical degree. The student may be pursuing a law degree. The user's age may be in the range of about 15 years old to about 30 years old.

### Computer Systems

The disclosure provides systems that implement the methods described herein. Systems may assess a mental health status and/or trajectory of a user. In some embodiments, the systems are computer systems comprising one or more processors configured to collect data from a user (e.g., passive data, active data, self-reported data, external data, etc.). The one or more processors may be configured to analyze the data of the user with a machine learning model, which can assess and generate a mental health status and/or trajectory of the user. The mental health status may be related to one or more clinical and/or sub-clinical conditions. The mental health status may include a mental health trajectory. In some embodiments, the computer system comprises a software module able to generate one or more resources related to the mental health status and/or trajectory of the user and curated to the user based on information on the user (e.g., demographics). The systems may include a smart device of a user that is communicatively coupled to the computer system. The smart device may include an app configured to display, on a graphical user interface (GUI), questions, prompts, mental health status, mental health trajectory, resources, or any combination thereof for the user. Systems may include a scalable data infrastructure. The data infrastructure may allow multimodal data (e.g., passive data, active data, self-reported data, external data, etc.), to be collected and derived into usable features. The data infrastructure may allow multimodal data (e.g., passive data, active data, self-reported data, external data, etc.), to be collected and organized into data sets and or feature sets for use in statistical analysis and/or machine learning pipelines.

Disclosed herein, in some embodiments, are computer systems configured to collect data for the user and assess a mental health status of the user. In some embodiments, the user is a student. The student may be a high school student, college student, or graduate student. In some embodiments, the computer system may include one or more processors configured to execute instructions for performing the methods. In some embodiments, one or more processors are configured to analyze markers for the user, such as passive data, active data, self-reported data, and external data. The computer systems have one or more software modules for collecting data, such as passive data, active data, self-reported data, and external data and generating or updating a mental health status of a user.

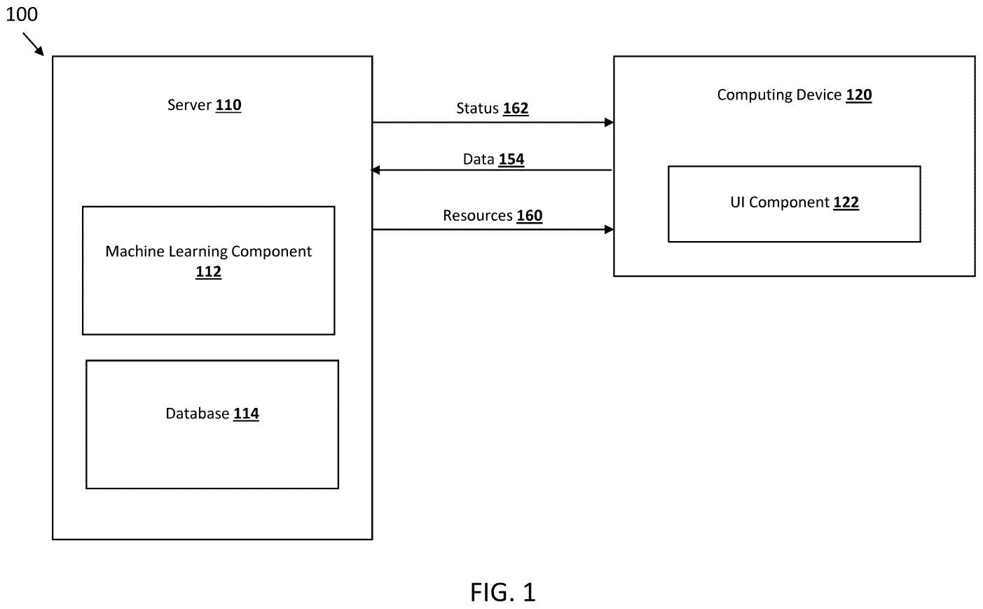

FIG. 1 depicts an example of a computer system 100 for assessing a mental health status or a change thereof with one or more resources for a user. In this depicted example, system 100 includes server 110 and computing device 120. In this depicted embodiment, server 110 further includes machine learning component 112 and database 114. In this depicted embodiment, server 110 is configured to communicate (e.g., send or collect information) with computing device 120. In this depicted embodiment, server 110 is configured to collect data 154 from computing device 120. In some embodiments, server 110 may be configured to collect data 154 from one or more devices (e.g., a smart phone and a tablet of the user). In this depicted embodiment, computing device 120 further comprises UI component 122. In this depicted embodiment, computing device 120 is configured to communicate (e.g., send or collect information) with server 110. In this depicted embodiment, computing device 120 is configured to send data 154 from computing device 120. In some embodiments, data 154 may include all or part of data. In some embodiments, the computing device 120 may be associated with a user. In this depicted embodiment, UI component 122 of computing device 120 may include one or more user interfaces. The one or more user interfaces may be configured to collect input.

Machine learning component 112 may be configured to generate one or more resources for a user based on collected data 154. Machine learning component 112 may include one or more machine learning models. The one or more machine learning models may include a random forest machine learning model or a gradient boosted machine learning model. In some embodiments, the machine learning model comprises an unsupervised machine learning model. In some embodiments, the unsupervised machine learning model comprises a clustering algorithm, such as a K-means clustering, centroid-based clustering algorithm, density-based clustering algorithm, distribution-based clustering algorithm, or a hierarchical clustering algorithm. In some embodiments, the one or more machine learning models are trained using a machine learning algorithm selected from any one of principal component analysis (PCA), uniform manifold approximation and projection (UMAP), variational autoencoder (VAE), support vector machines (SVM), recurrent neural networks (RNNs), long short-term memory networks (LSTMs), time series, transformers, large language models, diffusion models, convolutional neural networks, other artificial neural networks, decision trees or any combination thereof.

Machine learning component 112 may use the collected data 154 from the user to assess a mental health status of the user. Machine learning component 112 may use the collected data 154 from the user to generate and provide resources to the user. In some embodiments, database 114 may include a baseline for the user. In some embodiments, the database 114 may include a plurality of mental health statuses (e.g., data) for a plurality of users, where the plurality of mental health statuses includes the mental health status for the user.

In assessing the mental health status, machine learning component 112 may use collected data 154 for the user. In generating the resources, machine learning component 112 may use collected data 154. As described above, the mental health status may include a clinical condition, a sub-clinical condition, or a mental health trend. In some embodiments, at least one machine learning model of the one or more machine learning models of machine learning component 112 may collect the data 154 of the user as input and may output one or more mental health statuses.

In this depicted embodiment, the server 110 provides a resource 160 to the computing device 120. In some embodiments, the computing device 120 may display resource 160 on the user interface component 122. In some embodiments, at least a second machine learning model of machine learning component 112 may update the mental health status of the user based on data 154. The updated mental health status may then be used to determine resources.

In some embodiments, the process for generating a mental health status 162 may include a processing device (e.g., server 110) collecting data 154 for the user. In some embodiments, the process for generating a resource 160 may include a processing device (e.g., server 110) collecting a mental health status 162 for a user. In some embodiments, the mental health status 162 of a user is updated following server 110 collecting new data 154 for the user. In some embodiments, resource 160 provided to a user is updated following server 110 collecting new data 154 for the user. Machine learning component 112, using one or more machine learning models, may analyze mental health status 162 and data 154 to generate resource 160.

### Machine Learning

Machine learning models (e.g., model, ML model, Al, Al model) may have a training phase and an inference phase. During the training phase the model may learn using methods described below. During inference the machine learning model may be stopped from learning. When a model is used that has already been trained it may be called pretrained. The term “pretrained” makes no assumption about the performance of the model only that it has undergone some training. Multiple rounds of training may be performed. When a pretrained model goes through a subsequent round of training it may update the model through a method such as continuous learning, fine tuning, transfer learning or other methods.

A machine learning model such as those disclosed here may comprise hyperparameters (such as layer size, number of layers, choice of optimizer, learning rate, etc.), parameters (such as weights, biases, or coefficients), one or more processing steps (such as layers), and may produce one or more outputs and have one or more inputs. Hyperparameters may be optimized, in a process called hyperparameter optimization. Hyperparameters may be set during training and not change. Parameters may be changed during training. During training the machine learning model may calculate a loss useful for calculating the error between the real output of the model and the expected output of the model (for example, labels). A loss may measure a portion of the model, such as information in the model and/or the learned distribution of samples. Some set of the model parameters may be updated based at least in part on the loss calculation. The model may perform multiple rounds, or epochs, of training wherein an input or set of inputs is given and processed by the model which may produce an output or set of outputs which may then the basis for updating the weights. The updated weights may be used in the next epoch. Some training may comprise more steps. Training may occur in different environments such as supervised, unsupervised, semi-supervised, self-supervised or some combination thereof.

In a supervised environment the expected output may be provided for each input during training. The training data may have labels associated with each sample of the training data. The labels are an indication of the desired output of the model when the corresponding input is given. For example, a model may be trained using a set of data samples such as sleep data and/or activity data for one or more users as the input and the labels may be a set of interventions that were recommended by a physician that correspond to the input data samples, the model is then trained to model the mapping of the sleep and/or activity data to the labels.

In an unsupervised method the training set does not have corresponding labels. In some cases the input is the desired output of the model and may be used in place of a label. In other cases, the desired output is communicated through a score which may be related to some other output indication. For example, a model may use data samples such as sleep and activity data as input, the model may be trained to learn a latent representation (sometimes referred to as an encoding) that maps the input to a lower dimensional space and which may be used to generate the input. Such an encoding may be found in models trained using other training methods, such as supervised, self-supervised or semi-supervised.

The model may be trained using self supervised learning (SSL). SSL may use no labels. SSL may use some labels. Self-supervised methods may generate implicit labels from the unstructured data. In SSL, tasks may fall into two categories: pretext tasks and downstream tasks. In a pretext task, SSL may be used to train an Al system to learn meaningful representations of unstructured data. Those learned representations can be subsequently used as input to a downstream task, like a supervised learning task or reinforcement learning task. The reuse of a pre-trained model on a new task is referred to as “transfer learning.”

SSL may be used in the training of a diverse array of sophisticated deep learning architectures for a variety of tasks, from transformer-based large language models (LLMs) like BERT and GPT to image synthesis models like variational autoencoders (VAEs) and generative adversarial networks (GANs) to computer vision models like SimCLR and Momentum Contrast (MoCo). These methods may use other types of learning such as semi-supervised learning, supervised learning, and/or unsupervised learning.

Semi supervised may combine unsupervised and supervised tasks by using labeled and unlabeled data. In some cases, there may be datasets where some samples are labeled and others are not. In these cases, it may be desirable to have a fully labeled dataset but producing labels for large datasets is time consuming and expensive. Semi supervised learning first trains on the labeled data of the set of data and may then be used to produce pseudo-labels, or labels that are not validated.

### Labels/Ground Truth

Labels may be in various forms. Labels may be in a continuous range, for example 0 to 1. A label may use a confidence threshold. A confidence value may be associated with a label. A confidence value above a confidence threshold may be used along with the labeled data to retrain the model to improve the overall performance of the model. A label may be binary. Labels may be ordinal. Labels may be cardinal. Labels may be discrete. Labels may be vectors. Labels may be scalars. Labels may be incomplete (e.g., not all labels are present).

### Classification

A machine learning model may be trained as a classifier. A classifier may perform multiclass classification where more than one class is indicated. A classifier may be a multiclass multilabel, where more than one class may be output as present at one time. This may be useful in settings where classes may co-exist in the input. For example, an image segmentation model or object detection model may indicate the presence of multiple objects in an image and output an indication in its output for each of the detected objects. This may also be useful when the model is used to detect either multiple classes in the input and/or where some other label is desired such as a contextual output.

### Regression

A machine learning model may be trained as a regression model. A regression model may be used in a predictive fashion, whereas a classifier is used to place input or portions of input into classes that are predefined. Regression models may take an input and output a continuous value as a prediction or forecast score. As an example, a regression model may take an image and predict a desired set of values describing a shape of a new object to be placed in the image. In this example the output, or a portion of the output, of a regression may be used as an input to another model.

Once training is completed, a model may be used to infer on a set of inputs. The model output may be the desired output for the use of the model or there may be some portion of the model that is used for a desired output different than the output that was used during training time. At inference time the model's weights may be static.

### Retraining, Transfer Learning, Fine Tuning

A model may be trained. Model training may involve an optimization step wherein model parameters (such as weights or biases) may be altered based on the optimizer. Model training may involve a loss function which calculates a score based on the output of the model and the expected output of the model (such as a ground truth or labels). Model training may involve a dataset. The dataset may be split into one or more subsets. The subsets may be of different sizes. The subsets may be used for training, validation, testing or any combination thereof.

A model may be trained more than once. A model may be trained one a different dataset than was used in a previous training round (e.g., transfer learning, fine-tuning of the model, integration of the model into a larger model, continuous training, or some combination thereof). During training, whether in the first round or subsequent rounds, a subset of the model parameters may be untrainable during fine-tuning.

During fine-tuning a trained model may be trained on a different set of data, a subset of the original data or some combination thereof. Fine-tuning may cause the model to improve its performance on a given task or subtask. During transfer learning a model may be trained to improve performance on a task similar to the task the model was previously trained on. During transfer learning a model may be trained to improve performance on a task not similar to the task the model was previously trained on. During transfer learning a model may be trained to learn a different task it was not previously trained for, for example a model may be trained to generate a treatment plan based on a set of training data in the first round of training, in a subsequent round of training the model may then be trained to generate a treatment plan based/fine-tuned on the users data in order to generate an improved plan based on the specifics of the user data which may vary from that of the original training data.

### Neural Networks

### General Neural Networks

A model may comprise one or more neural networks. A neural network may use artificial neurons an individual processing units. These artificial neurons may comprise at least one of an input, a set of weights, a set of biases, a summation step and an activation function (for example, rectified linear units (ReLu), exponential linear activation, sigmoid function, linear activation, leaky relu, softmax, tanh, or others) or any combination thereof. Multiple artificial neurons may be used to create a layer of neurons that takes in the same input and outputs a number of values equal to the number of neurons in that layer. A neural network may be composed of multiple layers. Layers may take as input data, or output from other layers or some other values such as a random value. Layers may be smaller, larger or the same size as the input they take. Layers may be of various types such as, but not limited to, the following layer types; dense, convolutional, pooling, recurrent, preprocessing, normalization, regularization, attention, reshaping, merging, or activation. When a layer's output is received as input by another layer the two layers are connected. Layers may be connected to any layer that follows.

### Neural Network Architectures

Layer connectivity may define the model's architecture. Choice of a model's architecture may be directed by the task being carried out by the layer or set of layers. Layer architectures may then be described by their function. Some examples of architectures are, feed-forward networks, recurrent neural networks (RNN), long short-term memory (LSTM), echo networks, diffusion models, transformers, visual geometry group (VGG), graph neural networks (GNN), encoders, variational autoencoders (VAE), UNET, and generative adversarial networks. Networks are generally agnostic to the layer types used in them and may comprise multiple layer types. As an example, a convolutional neural network (CNN) may be a feed forward network comprising convolutional layers as well as pooling, and flattening layers, this is only an example though and CNNS may have different architectures or layer compositions. Architectures may also be combined in one model.

### System for Training a Machine Learning Model

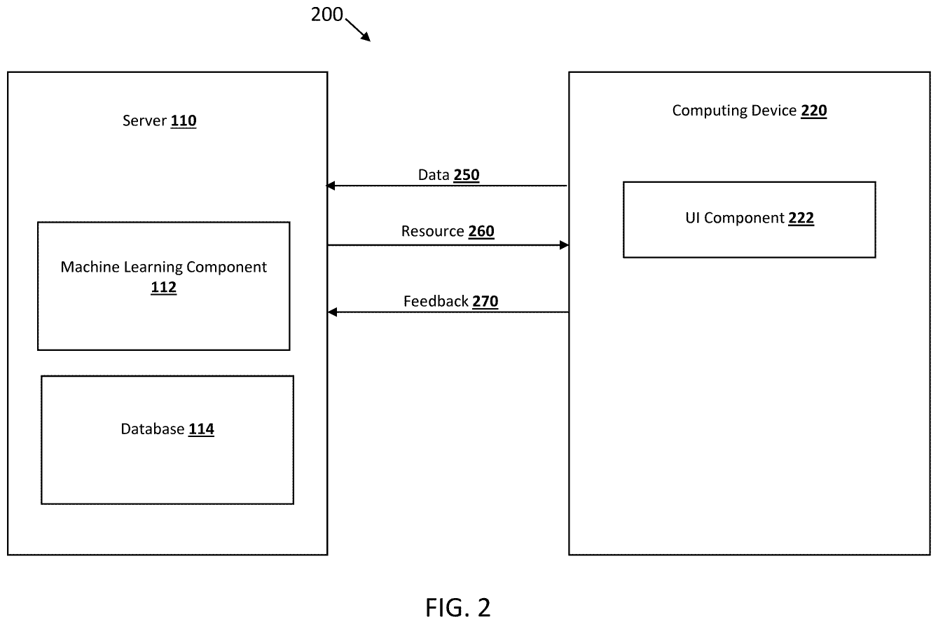

Also provided are systems for training a machine learning model. FIG. 2 depicts an example of a computer system 200 for training one or more machine learning models to assess a mental health status and/or provide a resource. In this depicted example, system 200 includes server 110 and computing device 220.

In this depicted example, server 110 further includes machine learning component 112 and database 114. In this depicted embodiment, server 110 is configured to communicate (e.g., send or collect information) with computing device 220. In this depicted embodiment, server 110 may collect training data regarding a user from computing device 220. In some embodiments, the training data includes a plurality of training data. In this depicted embodiment, server 110 is configured to collect training data 250.

In some embodiments, machine learning component 112 includes one or more machine learning models configured to collect the plurality of training data 250 to generate a mental health status and/or resource. In some embodiments, the plurality of training data includes one or more attributes relating to the mental health status of the and the training data 250 includes training passive data, active data, self-reported data, and external data. Based on the plurality of training data, machine learning component 112 may assess a mental health status and/or provide a resource.

Computing device 220 may further be configured to provide feedback 270 regarding the mental health status and/or resource 260. In some embodiments, feedback 270 regarding the mental health status and/or resource 260 may include revised mental health status and/or resource. Server 110 may further be configured to collect feedback 270. In some embodiments, the one or more machine learning models of the machine learning component 112 may adjust one or more parameters in response to the feedback 270, thereby training the one or more machine learning models of machine learning component 112.

### User Interfaces

In assessing a user for a mental health status, the system may use one or more graphical user interfaces (e.g., through UI component 122 of FIG. 1 or UI component 222 of FIG. 2). A processing device (e.g., computing device 120 of FIG. 1 or computing device 220 of FIG. 2) may display graphical user interfaces. In some embodiments, graphical user interfaces may display one or more aspects of self-reported and/or active data. In some embodiments, a user may use the graphical user interfaces to input self-reported and/or active data to the processing device. In some embodiments, the user input may be used to augment or edit the data. The graphical user interface may display a mental health status and/or trajectory and one or more resources for the user.

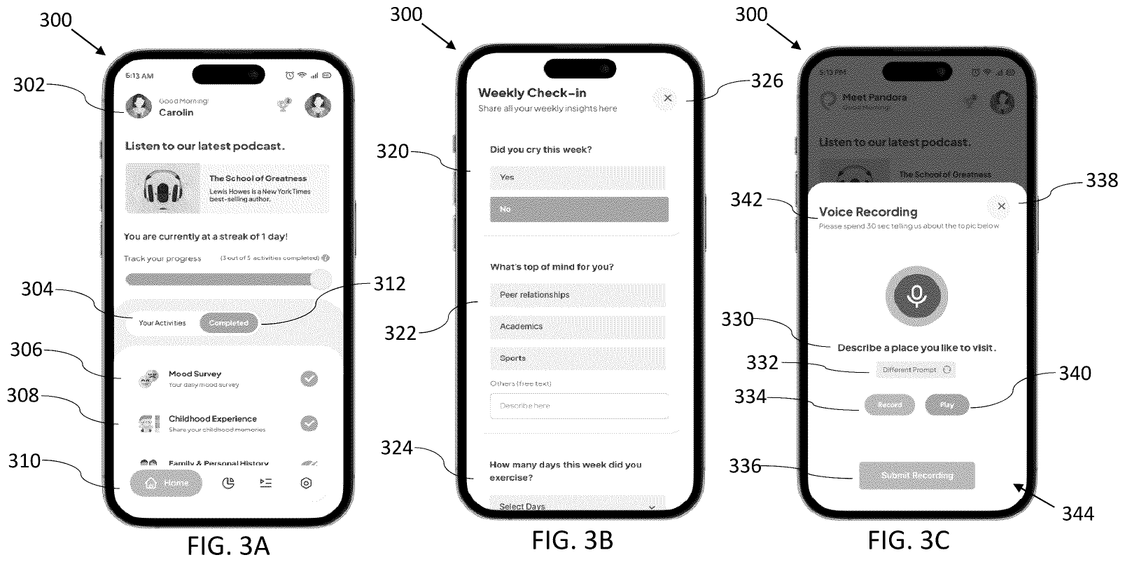

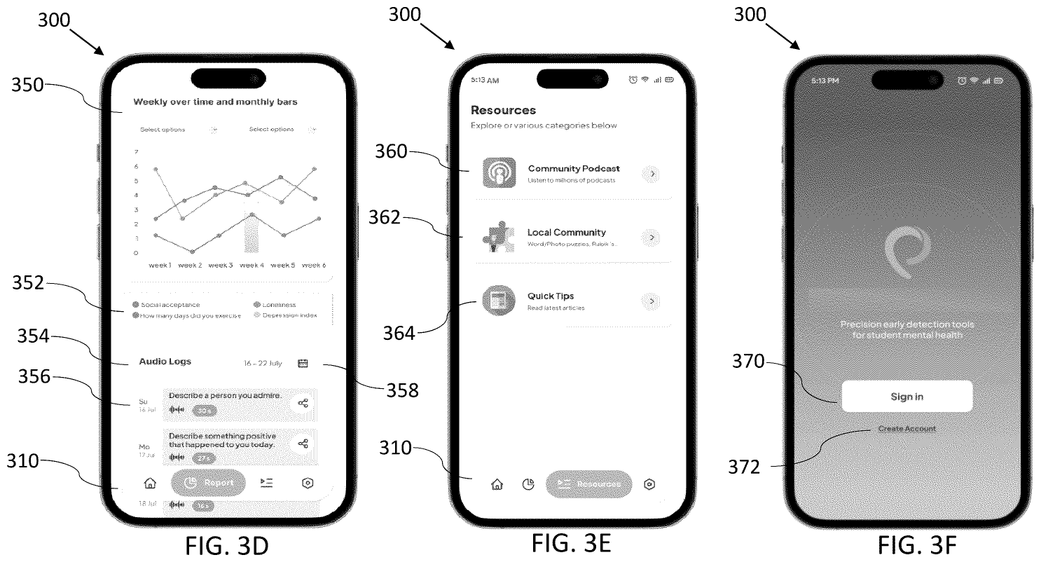

FIG. 3A-F depicts an example user interface 300 including a plurality of interactive elements for viewing a mental health status, a mental health trajectory, resources, entering self-reported or active data (for example, responding to a prompt or answering a question). In some embodiments, the interactive elements may be accessed through touch input, voice or sound input, electronic input, manual input (e.g., using a mouse to click on an interactive element), or another form of input. While certain inputs are listed above, these inputs are exemplary and other inputs may be used. In some embodiments, additional elements may be included that are not interactive.

FIG. 3A depicts an example of the “Home” screen for the app upon opening the app on a smart device (e.g., smart phone or tablet). In this depicted embodiment, a username and profile 302 are accessible. The “Home” screen also has options for toggling between views of the activities needing to be completed 304 and the activities that are completed 312. In this depicted embodiment, the activities that are completed 312 are shown on the screen as evident by the darkened background surrounding the “Completed” text. With the “Completed” selected, a list of activities that have been completed are displayed below the area to toggle between activities needing to be completed 304 and the activities that are completed 312. The activities completed include the mood survey 306, childhood experience 308, and others than can be brought into view by dragging the page up. At the very bottom GUI is a menu 310 containing four options. In this depicted embodiment, “Home” is surrounded by a darkened background and is accompanied by a house pictorial. The other options available in the menu include “Insights” depicted by a pie graph, “Resources” depicted by a paragraph pictorial, and “Settings”.

FIG. 3B depicts an example of a weekly check-in page for completion by the user. In this depicted embodiment, the weekly check-in has not been completed and would have been listed on the home screen depicted in FIG. 3A when the activities needing to be completed (e.g., activities needing to be completed 304 from FIG. 3A) was selected using the toggle. In this depicted embodiment, there are three questions with different response options for the user to provide self-reported data. The first question 320 includes two responses the user can choose from to answer the question. In this depicted embodiment, the user has responded “No” as evident by the darker background as compared to the “Yes” background for this question. The following question 322 has not been answered. In this depicted embodiment, the user may answer question 322 by selecting one of three prefilled answers or they may choose to enter their own answer. The last question 324 visible on the screen has not been answered. In this depicted embodiment, the user may choose from a drop-down list to select an answer to this question 324. At any time, the user may exit out of the page and return to the home screen by selecting the “X” 326.

FIG. 3C depicts an example of a voice response page for completion by the user. In this depicted embodiment, the voice response page would have been listed on the home screen depicted in FIG. 3A when the activities needing to be completed (e.g., activities needing to be completed 304 from FIG. 3A) was selected using the toggle. The activity and general instructions 342 are found at the top of the splash screen 344. The prompt 330 for use in the response may be changed 332. In this depicted embodiment, the user may record 334 their prompt. Once a user completes their prompt, they may play it back 340. The user may submit their recording 336 or they may decide to exit the splash screen 344 by selecting the “X; 338.

FIG. 3D depicts an example of the “Report” screen for the app. In this depicted embodiment, menu 310 has “Report” surrounded by a darkened background and is accompanied by a pie graph. The other menu 310 options are also available including “Home’, “Resources’, and “Settings” all represented by their pictorial. In this depicted embodiment, a graph 350 is shown representing four categories 352 including social acceptance, number of days in a week the user exercised, loneliness, and a depression index. The graph 350 displays the information over 6 weeks. In this depicted embodiment, the user has two drop down menus to customize the graph 350. The “Report” screen as depicted in FIG. 3D also allows a user to review their audio logs 354. The user may customize the dates 358 for the displayed audio logs. The audio logs listed provide details such as the date the recording was made, the length of the recording, the prompt of the recording, and allows the user to share the recording as shown at 356.

FIG. 3E depicts an example of the “Resources” screen. In this depicted embodiment, menu 310 has “Resources” surrounded by a darkened background and is accompanied by a paragraph pictorial. The other menu 310 options are also available including “Home’, “Resources’, and “Settings” all represented by their pictorial. In this depicted embodiment, the user has three resources available (360, 362, and 364). The three resources are broken up by category and may be selected to generate a new view specific to the selected resources category. In this depicted embodiment, the resource categories include community podcast 360, local community 362, and quick tips 364.

FIG. 3F depicts an example of the log-in and sign-up GUI. In this depicted embodiment, a user may select to sign in 370 or create account 372.

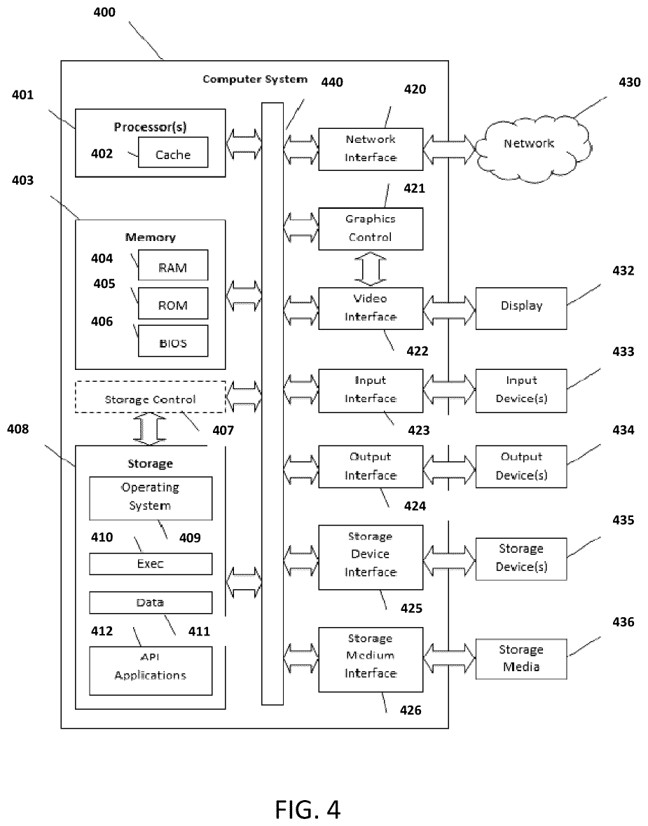

Referring to FIG. 4, a block diagram is shown depicting an exemplary machine that includes a computer system 400 (e.g., a processing or computing system) within which a set of instructions can execute for causing a device to perform or execute any one or more of the aspects and/or methodologies for static code scheduling of the present disclosure. The components in FIG. 4 are examples only and do not limit the scope of use or functionality of any hardware, software, embedded logic component, or a combination of two or more such components implementing particular embodiments.

Computer system 400 may include one or more processors 401, a memory 403, and a storage 408 that communicate with each other, and with other components, via a bus 440. The bus 440 may also link a display 432, one or more input devices 433 (which may, for example, include a keypad, a keyboard, a mouse, a stylus, a finger, etc.), one or more output devices 434, one or more storage devices 435, and various tangible storage media 436. All of these elements may interface directly or via one or more interfaces or adaptors to the bus 440. For instance, the various tangible storage media 436 can interface with the bus 440 via storage medium interface 426. Computer system 400 may have any suitable physical form, including but not limited to one or more integrated circuits (Ics), printed circuit boards (PCBs), mobile handheld devices (such as mobile telephones, smartphones, tablets, or PDAs), laptop or notebook computers, distributed computer systems, computing grids, or servers.

Computer system 400 includes one or more processor(s) 401 (e.g., central processing units (CPUs), general purpose graphics processing units (GPGPUs), or quantum processing units (QPUs)) that carry out functions. Processor(s) 401 optionally contains a cache memory unit 402 for temporary local storage of instructions, data, or computer addresses. Processor(s) 401 are configured to assist in execution of computer readable instructions. Computer system 400 may provide functionality for the components depicted in FIG. 4 as a result of the processor(s) 401 executing non-transitory, processor-executable instructions embodied in one or more tangible computer-readable storage media, such as memory 403, storage 408, storage devices 435, and/or storage medium 436. The computer-readable media may store software that implements particular embodiments, and processor(s) 401 may execute the software. Memory 403 may read the software from one or more other computer-readable media (such as mass storage device(s) 435, 436) or from one or more other sources through a suitable interface, such as network interface 420. The software may cause processor(s) 401 to carry out one or more processes or one or more steps of one or more processes described or illustrated herein. Carrying out such processes or steps may include defining data structures stored in memory 403 and modifying the data structures as directed by the software.

The memory 403 may include various components (e.g., machine readable media) including, but not limited to, a random access memory component (e.g., RAM 404) (e.g., static RAM (SRAM), dynamic RAM (DRAM), ferroelectric random access memory (FRAM), phase-change random access memory (PRAM), etc.), a read-only memory component (e.g., ROM 405), and any combinations thereof. ROM 405 may act to communicate data and instructions unidirectionally to processor(s) 401, and RAM 404 may act to communicate data and instructions bidirectionally with processor(s) 401. ROM 405 and RAM 404 may include any suitable tangible computer-readable media described below. In one example, a basic input/output system 406 (BIOS), including basic routines that help to transfer information between elements within computer system 400, such as during start-up, may be stored in the memory 403.

Fixed storage 408 is connected bidirectionally to processor(s) 401, optionally through storage control unit 407. Fixed storage 408 provides additional data storage capacity and may also include any suitable tangible computer-readable media. Storage 408 may be used to store operating system 409, executable(s) 410, data 411, applications 412 (application programs), and the like. Storage 408 can also include an optical disk drive, a solid-state memory device (e.g., flash-based systems), or a combination of any of the above. Information in storage 408 may, in appropriate cases, be incorporated as virtual memory in memory 403.

In one example, storage device(s) 435 may be removably interfaced with computer system 400 (e.g., via an external port connector (not shown)) via a storage device interface 425. Particularly, storage device(s) 435 and an associated machine-readable medium may provide non-volatile and/or volatile storage of machine-readable instructions, data structures, program modules, and/or other data for the computer system 400. In one example, software may reside, completely or partially, within a machine-readable medium on storage device(s) 435. In another example, software may reside, completely or partially, within processor(s) 401.

Bus 440 connects a wide variety of subsystems. Herein, reference to a bus may encompass one or more digital signal lines serving a common function, where appropriate. Bus 440 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 400 may also include an input device 433. In one example, a user of computer system 400 may enter commands and/or other information into computer system 400 via input device(s) 433. Examples of an input device(s) 433 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 433 may be interfaced to bus 440 via any of a variety of input interfaces 423 (e.g., input interface 423) including, but not limited to, serial, parallel, game port, USB, FIREWIRE, THUNDERBOLT, or any combination of the above.

In particular embodiments, when computer system 400 is connected to network 430, computer system 400 may communicate with other devices, specifically mobile devices and enterprise systems, distributed computing systems, cloud storage systems, cloud computing systems, and the like, connected to network 430. Communications to and from computer system 400 may be sent through network interface 420. For example, network interface 420 may collect incoming communications (such as requests or responses from other devices) in the form of one or more packets (such as Internet Protocol (IP) packets) from network 430, and computer system 400 may store the incoming communications in memory 403 for processing. Computer system 400 may similarly store outgoing communications (such as requests or responses to other devices) in the form of one or more packets in memory 403 and communicated to network 430 from network interface 420. Processor(s) 401 may access these communication packets stored in memory 403 for processing.

Examples of the network interface 420 include, but are not limited to, a network interface card, a modem, and any combination thereof. Examples of a network 430 or network segment 430 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 430, may employ a wired and/or a wireless mode of communication. In general, any network topology may be used.

Information and data can be displayed through a display 432. Examples of a display 432 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 432 can interface to the processor(s) 401, memory 403, and fixed storage 408, as well as other devices, such as input device(s) 433, via the bus 440. The display 432 is linked to the bus 440 via a video interface 422, and transport of data between the display 432 and the bus 440 can be controlled via the graphics control 421. In some embodiments, the display is a video projector. In some embodiments, the display is a head-mounted display (HMD) such as a VR headset. In further embodiments, suitable VR headsets include, by way of Examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices.

In addition to a display 432, computer system 400 may include one or more other peripheral output devices 434 including, but not limited to, an audio speaker, a printer, a storage device, and any combinations thereof. Such peripheral output devices may be connected to the bus 440 via an output interface 424. Examples of an output interface 424 include, but are not limited to, a serial port, a parallel connection, a USB port, a FIREWIRE port, a THUNDERBOLT port, and any combinations thereof.

Computer system 400 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of Examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of Examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of Examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of Examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of Examples, Apple TV®, Roku®, Boxee®, Google TV®, Google Chromecast®, Amazon Fire®, and Samsung® HomeSync®. Those of skill in the art will also recognize that suitable video game console operating systems include, by way of Examples, Sony® PS3®, Sony® PS4®, Sony® PS5®, Microsoft® Xbox 360®, Microsoft® Xbox One, Microsoft® Xbox Series X, Microsoft® Xbox Series S, Nintendo® Wii®, Nintendo® Wii U®, Nintendo® Switch™, and Ouya®.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of Examples, relational, non-relational, object oriented, associative, XML, and document-oriented database systems. In further embodiments, suitable relational database systems include, by way of Examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous JavaScript and XML (AJAX), Flash® ActionScript, JavaScript, or Silverlight®. In some embodiments, a web application is written to some extent in a server-side coding language such as Active Server Pages (ASP), ColdFusion®, Perl, Java™ JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of Examples, Adobe® Flash®, HTML 5, Apple® QuickTime®, Microsoft® Silverlight®, Java™, and Unity*.

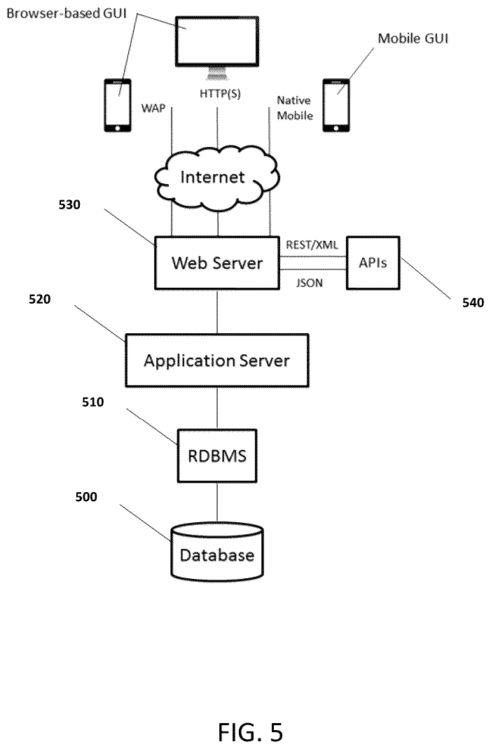

Referring to FIG. 5, in a particular embodiment, an application provision system comprises one or more databases 500 accessed by a relational database management system (RDBMS) 510. Suitable RDBMSs include Firebird, MySQL, PostgreSQL, SQLite, Oracle Database, Microsoft SQL Server, IBM DB2, IBM Informix, SAP Sybase, Teradata, and the like. In this embodiment, the application provision system further comprises one or more application servers 520 (such as Java servers, .NET servers, PHP servers, and the like) and one or more web servers 530 (such as Apache, IIS, GWS and the like). The web server(s) optionally expose one or more web services via app application programming interfaces (APIs) 540. Via a network, such as the Internet, the system provides browser-based and/or mobile native user interfaces.

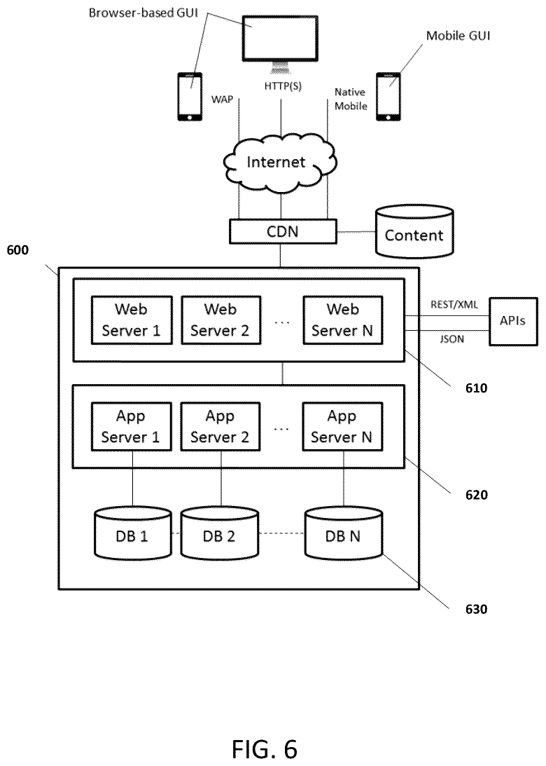

Referring to FIG. 6, in a particular embodiment, an application provision system alternatively has a distributed, cloud-based architecture 600 and comprises elastically load balanced, auto-scaling web server resources 610 and application server resources 620 as well synchronously replicated databases 630.

### Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of Examples, C, C++, C#, Dart, Objective-C, Java™ JavaScript, Kotlin, Pascal, Object Pascal, Python™, Ruby, Rails, Swift, VB.NET, WML, and XHTML/HTML with or without CSS, or combinations thereof.

Suitable mobile application development frameworks or environments are available from several sources. Commercially available development frameworks or environments include, by way of Examples, AirplaySDK, alcheMo, Appcelerator®, Celsius, Bedrock, Flash Lite, Flutter, .NET Compact Framework, Rhomobile, and WorkLight Mobile Platform. Other development frameworks or environments are available without cost including, by way of Examples, Lazarus, MobiFlex, MoSync, and Phonegap. Also, mobile device manufacturers distribute software developer kits including, by way of Examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of Examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® Dsi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of Examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of Examples, C++, Delphi, Java™, PHP, Python™, and VB .NET, or combinations thereof.

Web browsers (also called Internet browsers) are software applications, designed for use with network-connected computing devices, for retrieving, presenting, and traversing information resources on the World Wide Web. Suitable web browsers include, by way of Examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of Examples, handheld computers, tablet computers, netbook computers, subnotebook computers, smartphones, music players, personal digital assistants (PDAs), and handheld video game systems. Suitable mobile web browsers include, by way of Examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods include software, server, and/or database modules, or use of the same. In view of the disclosure provided herein, software modules are created by techniques known to those of skill in the art using machines, software, and languages known to the art. The software modules are implemented in a multitude of ways. In various embodiments, a software module comprises a file, a section of code, a programming object, a programming structure, a distributed computing resource, a cloud computing resource, or combinations thereof. In further various embodiments, a software module comprises a plurality of files, a plurality of sections of code, a plurality of programming objects, a plurality of programming structures, a plurality of distributed computing resources, a plurality of cloud computing resources, or combinations thereof. In various embodiments, the one or more software modules comprise, by way of Examples, a web application, a mobile application, a standalone application, and a distributed or cloud computing application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Databases

In some embodiments, the platforms, systems, media, and methods include one or more databases, or use of the same. In view of the disclosure provided herein, those of skill in the art will recognize that many databases are suitable for storage and retrieval of user information. In various embodiments, suitable databases include, by way of Examples, relational databases, non-relational databases, object-oriented databases, object databases, entity-relationship model databases, associative databases, XML databases, document-oriented databases, and graph databases. Further Examples include SQL, PostgreSQL, MySQL, Oracle, DB2, Sybase, DynamoDC, and MongoDB. In some embodiments, a database is Internet-based. In further embodiments, a database is web-based. In further embodiments, a database is cloud computing based. In a particular embodiment, a database is a distributed database. In other embodiments, a database is based on one or more local computer storage devices.

### Data Transmission

The methods may be performed at one or more locations. Facility locations may be in multiple geographic regions, such as multiple countries, states, provinces, counties, cities, regions, territories, and the like. In some instances, steps of the methods are performed in different geographic regions. In some instances, steps for collecting data are performed in different geographic regions. In some instances, a step for collecting data is performed in a geographic region that differs from a step for extracting a feature. In some instances, a computer network performing steps of the methods is distributed across geographic regions. In some instances, data processing and analyses is distributed across geographic regions. In some embodiments, one data is transferred from one or more geographic regions to one or more other geographic regions.

In some embodiments, any step of any method described herein is performed by a software program or module on a computer. In additional or further embodiments, data from any step of any method described herein is transferred to and from facilities located within the same or different countries, including analysis performed in one facility in a particular location and the data shipped to another location or directly to a user in the same or a different country. In additional or further embodiments, data from any step of any method described herein is transferred to and/or collected from a facility located within the same or different countries, including analysis of a data input performed in one facility in a particular location and corresponding data transmitted to another location, or directly to an user, such as data related to the mental health status, resources, or the like, in the same or different location or country.

### Business Methods Utilizing a Computer

The methods described herein may utilize one or more computers. The computer may be used for managing a user's information such as data, database management, analyzing data, storing data, billing, marketing, reporting results, storing results, or a combination thereof. The computer may include a monitor or other user interface for displaying data, results, billing information, marketing information (e.g., demographics), customer information, or data information. The computer may also include means for data or information input. The computer may include a processing unit and fixed or removable media or a combination thereof. The computer may be accessed by a user in physical proximity to the computer, for example via a keyboard and/or mouse, or by a user that does not necessarily have access to the physical computer through a communication medium such as a modem, an internet connection, a telephone connection, or a wired or wireless communication signal carrier wave. In some cases, the computer may be connected to a server or other communication device for relaying information from a user to the computer or from the computer to a user. In some cases, the user may store data or information collected from the computer through a communication medium on media, such as removable media. It is envisioned that data relating to the methods can be transmitted over such networks or connections for reception and/or review by a party. The collecting party can be but is not limited to the user, a health care provider, or a health care manager.

Information in a database for the purpose of one or more of the following may be used: customer management, customer service, billing, and sales.

The database may be accessible by a customer, medical professional, or other third party. Database access may take the form of electronic communication such as a computer or telephone. The database may be accessed through an intermediary such as a customer service representative, business representative, consultant, or medical professional. The availability or degree of database access may change upon payment of a fee for products and services rendered or to be rendered.

### EMBODIMENTS

The following are non-limiting examples of embodiments of the invention. Any of these exemplary embodiments may be combined with any other of the embodiments described here or elsewhere in the specification and claims.
Embodiment 1: A computer implemented method of training a model for assessing a mental health status or a change thereof of a user, the method comprising:
   a. collecting marker values of a population of test users from two or more data channels, selected from the group consisting of passive data channels, active data channels, self-reported data channels, and external data channels;
   b. extracting a set of features from the marker values; and
   c. training a model using the set of features, wherein the model assesses a mental health status based on the set of features.
Embodiment 2: A computer implemented method of assessing a mental health status or a change thereof of a user, the method comprising:
   a. collecting marker values of the user from two or more data channels; and
   b. using a model trained pursuant to the method of Embodiment 1 to assess a mental health status of the user.
Embodiment 3: The method of Embodiment 2, wherein the assessing comprises ongoing monitoring of the mental health status of the user.
Embodiment 4: The method of Embodiment 1, wherein the set of features comprises features from two or more of the data channels.
Embodiment 5: The method of Embodiment 1, wherein the set of features comprises features from three or more of the data channels.
Embodiment 6: The method of Embodiment 1, wherein the set of features comprises features from four of the data channels.
Embodiment 7: The method of any one of Embodiments 1, 2, 85, 87, 112, 117, 118, and 131, wherein the marker values comprise values from a passive data channel.
Embodiment 8: The method of Embodiment 7, wherein the marker values from the passive data channel are selected from the group consisting of location data, wearables data, and device usage data.
Embodiment 9: The method of Embodiment 8, wherein the device usage data is collected by the user uploading a screenshot of a previous week's application usage on a smartphone.
Embodiment 10: The method of Embodiment 8, wherein the device usage data is collected automatically from a smartphone of the user.
Embodiment 11: The method of Embodiment 8, wherein the marker values from the passive data channel comprise location data selected from the group consisting of location, time spent at location, location type, and location frequency.
Embodiment 12: The method of Embodiment 11, wherein the location is selected from the group consisting of home, gym, school, restaurant, bar, church, and other.
Embodiment 13: The method of Embodiment 8, wherein the marker values from a passive data channel comprise wearables data selected from the group consisting of a user's heartrate, body temperature, activity, sleep, respirations, menstrual status, stress level, and combinations thereof.
Embodiment 14: The method of Embodiment 13 wherein the wearables data comprises activity data and is selected from the group consisting of steps taken, floors climbed, intensity minutes, calories burned, and combinations thereof.
Embodiment 15: The method of Embodiment 11, wherein the wearables data comprises sleep data and is selected from the group consisting of bedtime, wake up time, sleep duration, quality of sleep, and combinations thereof.
Embodiment 16: The method of Embodiment 8, wherein the marker values from a passive data channel comprise device usage data selected from the group consisting of app usage, battery usage and charging, call frequency and duration, location tracking data, mental health-related internet searches, overall screen time, category specific screen time, physical activity levels (e.g., step counts), sleep patterns inferred from phone activity, social media usage patterns, text message frequency, typing speed and pressure, usage of mental health apps, voice tone and pitch analysis during calls, and frequency and content changes in photos and videos.
Embodiment 17: The method of Embodiment 16, wherein category specific screen time is selected from the group consisting of social, entertainment, educational, and informational.
Embodiment 18: The method of Embodiment 16, wherein device usage data is encoded.
Embodiment 19: The method of Embodiment 18, wherein the device usage data is encoded by extracting sentiment and not semantic content.
Embodiment 20: The method of Embodiment 18, wherein the device usage data is encoded by a token to randomize said device usage data.
Embodiment 21: The method of Embodiment 8, wherein the device usage data comprises data derived from one or more screenshots.
Embodiment 22: The method of Embodiment 21, wherein the data derived from one or more screenshots comprises phone usage.
Embodiment 23: The method of Embodiment 22, wherein the one or more screenshots further comprises application usage on a phone.
Embodiment 24: The method of Embodiment 21 wherein the data derived from one or more screenshots comprises health data from a health tracking application.
Embodiment 25: The method of Embodiment 1 or 2, wherein the marker values comprise values from an active data channel.
Embodiment 26: The method of Embodiment 25, wherein the marker values from an active data channel comprise voice values data.
Embodiment 27: The method of Embodiment 26, wherein the voice values data is selected from the group consisting of voice characteristics, speech characteristics, background noise characteristics, and combinations thereof.
Embodiment 28: The method Embodiments 26, wherein the voice values data comprises passive noise data.
Embodiment 29: The method of Embodiment 26, wherein the voice values data is selected from the group consisting of tone of voice, inflection of voice, word count, speech rate, intensity of voice, pitch, magnitude, phonetics, tempo-spectral, formant, glottal closure instance and combinations thereof.
Embodiment 30: The method of Embodiment 1 or 2, wherein the marker values comprise values from a self-reported data channel.
Embodiment 31: The method of Embodiment 30, wherein the self-reported data channel comprises values from self-reported data.
Embodiment 32: The method of Embodiment 30, wherein the marker values from a self-reported data channel comprise data from a questionnaire.
Embodiment 33: The method of Embodiment 32, wherein the questionnaire is completed by a user, a user's supervisor, a user's co-worker, a user's teacher, a user's counselor, a user's family member, a user's friend, or a combination thereof.
Embodiment 34: The method of Embodiment 32, wherein the questionnaire comprises questions related to demographic, family history, health history, impairments, hobbies, mental health history, family mental health history, academic history, romantic history, exercise details, drug and alcohol use and history, sleep, diet, emotional status and history, socialization, recurrent thoughts, physical and biological signs, or a combination thereof.
Embodiment 35: The method of Embodiment 32, wherein the questionnaire comprises demographic questions selected from the group consisting of age, sex, gender identity, sexual orientation, race, ethnicity, religion, or any combination thereof.
Embodiment 36: The method of Embodiment 30, wherein the marker values from a self-reported data channel comprises an emotional identifier.
Embodiment 37: The method of Embodiment 30, wherein the marker values from a self-reported data channel comprises a daily emotional identifier.
Embodiment 38: The method of Embodiment 1 or 2, wherein the marker values comprise values from an external data channel.
Embodiment 39: The method of Embodiment 38, wherein the values from an external data channel are selected from the group consisting of weather reports, local current events, and global current events.
Embodiment 40: The method of Embodiment 1, wherein the features are selected using summary statistics.
Embodiment 41: The method of Embodiment 1, wherein the features are selected based on a latent space.
Embodiment 42: The method of Embodiment 41 wherein the latent space is based on a transformation of the marker values into the latent space.
Embodiment 43: The method of Embodiment 1, wherein each feature of the set of features selected improves the assessment of the mental health status.
Embodiment 44: The method of Embodiment 1, wherein an absence of a marker value is one of the set of features.
Embodiment 45: The method of Embodiment 1, wherein the model is trained using a machine learning algorithm selected from principal component analysis (PCA), uniform manifold approximation and projection (UMAP), artificial neural networks (e.g. variational autoencoder (VAE), recurrent neural networks (RNNs), long short-term memory networks (LSTMs), and transformers), time series, and any combination thereof.
Embodiment 46: The method of Embodiment 2, wherein assessing a mental health status or a change thereof comprises assessing a change in mental health status of the user.
Embodiment 47: The method of Embodiment 2, wherein assessing a mental health status or a change thereof comprises assessing a baseline mental health status of the user.
Embodiment 48: The method of Embodiment 2, wherein assessing a mental health status or a change thereof comprises assessing a change in mental health status of the user relative to a baseline mental health status of the user.
Embodiment 49: The method of Embodiment 2, wherein assessing a mental health status or a change thereof comprises predicting a mental health trajectory of the user.
Embodiment 50: The method of Embodiment 2, wherein assessing a mental health status or a change thereof comprises calculating a probability of a mental health status of the user.
Embodiment 51: The method of Embodiment 2, comprising referring the user to a mental health resource.
Embodiment 52: The method of Embodiment 51, wherein the mental health resource is selected based on a model.
Embodiment 53: The method of Embodiment 52, wherein the model comprises a machine learning model.
Embodiment 54: The method of Embodiment 53, wherein the model is trained based on data from users of the computer implemented method of assessing a mental health status or a change thereof.
Embodiment 55: The method of any one of Embodiments 51-54, wherein the referring is done via a computing device or system.
Embodiment 56: The method of any one of Embodiments 51-54, wherein the mental health resource is delivered via a computing device or system.
Embodiment 57: The method of any one of Embodiments 51-54, wherein the mental health resource is selected based on a model that accounts for one or more of the following data types: mental health status of the user, sexual identity of the user, cultural background of the user, religious beliefs of the user, hobbies and interests of the user, location of the user, and combinations thereof.
Embodiment 58: The method of any one of Embodiments 51-54, further comprising assessing use by the user of the mental health resources.
Embodiment 59: The method of Embodiment 58, wherein the assessing use by the user comprises assessing time the user is at a location of the mental health resource.
Embodiment 60: The method of Embodiment 58, wherein the assessing use by the user comprises assessing time the user interacts with a website of the mental health resource.
Embodiment 61: The method of Embodiment 58, wherein the assessing use by the user comprises assessing data generated from an app used to provide the mental health resource.
Embodiment 62: The method of Embodiment 58, wherein the assessing use by the user comprises assessing changes in the mental health status of the user.
Embodiment 63: The method of Embodiment 58, wherein the assessing use by the user comprises assessing feedback from the user regarding the mental health resource.
Embodiment 64: The method of Embodiment 2, further comprising reporting a clinical mental health status of the user.
Embodiment 65: The method of Embodiment 64, wherein reporting a clinical mental health status of the user comprises reporting an anxiety or depression status of the user.
Embodiment 66: The method of Embodiment 64 wherein reporting a clinical mental health status of the user comprises reporting a subclinical mental health status of the user.
Embodiment 67: The method of Embodiment 64 wherein reporting a clinical mental health status of the user comprises reporting acceptance or loneliness.
Embodiment 68: The method of Embodiment 1, wherein the population of test users are students.
Embodiment 69: The method of Embodiment 2, wherein the user is a student.
Embodiment 70: The method of Embodiment 68, wherein the student is a college student.
Embodiment 71: The method of Embodiment 1, wherein the population of test users are 18 to 24 years of age.
Embodiment 72: The method of Embodiment 2, wherein the user is 18 to 24 years of age.
Embodiment 73: The method of Embodiment 2, wherein the method further comprises using the model to predict a matriculation status of the user.
Embodiment 74: A method of improving retention of students or employees, the method comprising performing the method of Embodiment 51, on a set of students or employees and thereby improving retention of students or employees of the set.
Embodiment 75: The method of Embodiment 1, comprising continuously collecting marker values of the population of test users.
Embodiment 76: The method of Embodiment 2, comprising continuously collecting marker values of the user.
Embodiment 77: The method of Embodiment 75 or 76, wherein the continuous markers are collected over 3 months.
Embodiment 78: The method of Embodiment 75 or 76, wherein the continuous markers are collected over 6 months.
Embodiment 79: The method of Embodiment 75 or 76, wherein the continuous markers are collected over 1 year.
Embodiment 80: The method of Embodiment 75 or 76, wherein the continuous markers are collected over a semester.
Embodiment 81: The method of Embodiment 75 or 76, wherein the continuous markers are collected over 2 semesters.
Embodiment 82: The method of Embodiments 1-81, wherein the method further comprises encoding the marker values from the data channels.
Embodiment 83: The method of Embodiment 82, wherein the encoding comprises randomization of the marker values from the data channels.
Embodiment 84: The method of Embodiment 82, wherein the encoding comprises extracting sentiment content and discarding semantic content from the marker values from the data channels.
Embodiment 85: A computer implemented method for assessing a mental health status or a change thereof of a user, the method comprising:
   a. collecting marker values of a population of test users, the marker values drawn from at least two of: passive data, active data, self-reported data, and external data; and
   b. extracting a set of features from the marker values suitable for training a model.
Embodiment 86: The method of Embodiment 85, further comprising generating a list of curated resources.
Embodiment 87: A computer implemented method of training a model for assessing a mental health status or a change thereof of a user, the method comprising:
   a. collecting marker values of a population of test users, the marker values drawn from at least two of: passive data, active data, self-reported data, and external data; and
   b. training a model using the set of features, wherein the model assesses a mental health status based on the set of features.
Embodiment 88: The method of Embodiment 87, further comprising generating a list of curated resources.
Embodiment 89: A computer implemented method of extracting a health conclusion from location data, the method comprising:
   a. collecting location data on a user at one or more points in time;
   b. collecting data on a local condition at the user's position(s); and
   c. using a model to draw a health conclusion based on the location data and local-conditions data.
Embodiment 90: The method of Embodiment 89 or 112, wherein the local conditions comprise weather, news, local events, or any combination thereof.
Embodiment 91: The method of Embodiment 89 or 112, wherein the model considers multiple local conditions.
Embodiment 92: The method of Embodiment 89 or 112, wherein the model considers more than one user.
Embodiment 93: The method of Embodiment 89 or 112, further comprising generating a list of curated resources related to the health conclusion.
Embodiment 94: A computer implemented method of extracting a health conclusion from a user's voice, the method comprising:
   a. collecting from a first instance of a user's voice at least one of: a vocal cord characteristic, a speech characteristic, and a background noise characteristic;
   b. collecting from a second instance of a user's voice at least one of: a vocal cord characteristic, a speech characteristic, and a background noise characteristic; and
   c. using a model to draw a health conclusion based on characteristics collected from the first and second instances.
Embodiment 95: The method of Embodiment 94 or 113, wherein the instance is recorded by the user.
Embodiment 96: The method of Embodiment 94 or 113, wherein the instance is streamed by the user.
Embodiment 97: The method of Embodiment 94 or 113, further comprising generating a list of curated resources related to the health conclusion.
Embodiment 98: The method of Embodiment 94 or 113, further comprising determining a health conclusion of the user.
Embodiment 99: The method of Embodiment 98, wherein the health conclusion comprises a mental health status.
Embodiment 100: The method of Embodiment 99, wherein the mental health status comprises any one of healthy, depressive, anxious, or behavioral.
Embodiment 101: The method of Embodiment 94 or 113, wherein the characteristics comprise at least one of tone of voice, inflection of voice, word count, speech rate, intensity of voice, pitch, magnitude, phonetics, tempo-spectral, formant, glottal closure instance, or any combination thereof.
Embodiment 102: A computer implemented method of extracting a health conclusion from device usage data, the method comprising:
   a. collecting a user's device usage data at one or more points in time; and
   b. using a model to draw a health conclusion based on the device usage data.
Embodiment 103: The method of Embodiment 102 or 114, wherein the device usage data comprises a total of time a user spent on a device.
Embodiment 104: The method of Embodiment 102 or 114, wherein the device usage data comprises a total of time using one or more specific apps.
Embodiment 105: The method of Embodiment 102 or 114, wherein the device usage data comprises a total of time using one or more categories of apps.
Embodiment 106: The method of Embodiment 105, wherein the categories comprise any one of social, entertainment, educational, and informational.
Embodiment 107: A computer implemented method of extracting a health conclusion from a user's device, the method comprising:
   a. collecting, from a device at multiple points in time, data on a user's positioning, voice, and device usage; and
   b. using a model to draw a health conclusion based on the collected data.
Embodiment 108: A computer implemented method of providing health information for a user, the method comprising:
   a. collecting data about a person;
   b. using a model to draw a health conclusion based on the collected data; and
   c. providing at least one health resource option based on the health conclusion.
Embodiment 109: The method of Embodiment 108 or 116, wherein the data includes self-reported data.
Embodiment 110: The method of Embodiment 109, wherein the self-reported data includes private data.
Embodiment 111: The method of Embodiment 109, wherein the self-reported data includes encoded data.
Embodiment 112: A computer implemented method of training a model for generating a health conclusion from location data, the method comprising:
   a. collecting location data on a user at one or more points in time;
   b. collecting data on a local condition at the user's position(s);
   c. extracting marker values from the location data and local-conditions data; and
d. training a model to generate a health conclusion based on marker values.
Embodiment 113: A computer implemented method of training model to generate a health conclusion from a user's voice, the method comprising:
   a. collecting from a first instance of a user's voice at least one of a vocal cord characteristic, a speech characteristic, and a background noise characteristic;
   b. collecting from a second instance of a user's voice at least one of a vocal cord characteristic, a speech characteristic, and a background noise characteristic; and
   c. training a model to generate a health conclusion based on characteristics collected from the first and second recordings.
Embodiment 114: A computer implemented method of training a model to generate a health conclusion from device usage data, the method comprising:
   a. collecting a user's device usage data at one or more points in time; and
   b. training a model to generate a health conclusion based on the device usage data.
Embodiment 115: A computer implemented method of training a model to generate a health conclusion from a user's device, the method comprising:
   a. collecting, from a device at multiple points in time, data on a user's positioning, voice, and device usage; and
   b. training a model to generate a health conclusion based on the collected data.
Embodiment 116: The method of Embodiment 107 or 115, wherein the data on the device usage comprises an amount of time spent on the device.
Embodiment 117: The method of Embodiment 107 or 115, wherein the data on the device usage comprises an amount of time spent on one or more apps or categories thereof.
Embodiment 118: The method of Embodiment 107 or 115, wherein the data on the user's positioning comprises location data taken at multiple points in time.
Embodiment 119: The method of Embodiment 107 or 115, wherein the data on the user's positioning comprises a local condition at the user's position(s), such as weather, news, local events, or any combination thereof.
Embodiment 120: The method of Embodiment 107 or 115, wherein the data on the voice comprises first and second instances of the user's voice.
Embodiment 121: The method of Embodiment 120, wherein the first and second instances comprise at least one of a vocal cord characteristic, a speech characteristic, and a background noise characteristic.
Embodiment 122: A computer implemented method of training a model to generate health information for a user, the method comprising:
   a. collecting data about a user; and
   b. training a model to generate health information based on the collected data.
Embodiment 123: A computer implemented method of training a model for assessing a mental health status or a change thereof of a user, the method comprising:
   a. collecting marker values of a population of test users, the marker values drawn from at least two of: passive data, active data, self-reported data, and external data;
   b. extracting a set of features from the marker values; and
   c. training a model using the set of features, wherein the model assesses a mental health status based on the set of features.
Embodiment 124: A computer implemented method of training a model for assessing a performance outcome of a user, the method comprising:
   a. collecting marker values of a population of test users, the marker values drawn from at least two of: passive data, active data, self-reported data, and external data;
   b. extracting a set of features from the marker values; and
   c. training a model using the set of features, wherein the model assesses a performance outcome of the user based on the set of features.
Embodiment 125: A computer implemented method of predicting a performance outcome of a user, the method comprising:
   a. collecting marker values of a population of test users, the marker values drawn from at least two of: passive data, active data, self-reported data, and external data;
   b. extracting a set of features from the marker values; and
   c. predicting, using a trained model, a performance outcome of the user based on the set of features.
Embodiment 126: The method of Embodiment 124 or 125, wherein the performance outcome comprises attrition, grades, changes in major, taking longer to graduate, retention, or academic performance.
Embodiment 127: The method of any one of Embodiments 1-126, wherein the mental health condition comprises any one of depression, anxiety, and behavior.
Embodiment 128: The method of Embodiment 126, wherein the behavior comprises substance use and/or substance abuse.
Embodiment 129: The method of any one of Embodiments 1-128, wherein training the model comprises more than one user.
Embodiment 130: The method of any one of Embodiments 1-129, wherein the model considers some specific combination of features.
Embodiment 131: The method of any one of Embodiments 1-130, wherein the model considers changes in device usage data over time.
Embodiment 132: The method of any one of Embodiments 1-131, wherein the health conclusions comprise depression, anxiety, and/or behavior.
Embodiment 133: The method of any one of Embodiments 1-132 further comprising generating a list of curated resources related to the health conclusion.
Embodiment 134: The method of any one of Embodiments 1-133, further comprising identifying one or more curated resources related to the health conclusion.
Embodiment 135: The method of any one of Embodiments 1-134, further comprising using a predefined table and/or dataset that matches resource options to health conclusions.
Embodiment 136: The method of any one of Embodiments 1-135 further comprising selecting the resource option(s) to provide based on a ranking of available options.
Embodiment 137: The method of any one of Embodiments 1-136 further comprising updating the data collection, generating an updated health conclusion, and providing an updated resource option.
Embodiment 138: A computer implemented method of assessing a mental health status or a change thereof of a user, the method comprising:
   a. collecting marker values of the user from two or more data channels;
   b. extracting a set of features from the marker values;
   c. training a model using the set of features, wherein the model assesses a mental health status based on the set of features; and
d. using the model trained pursuant to the method of Embodiment 1 to assess a mental health status of the user.
Embodiment 139: A method of generating a treatment plan to a user, comprising:
   a. collecting a set of features from an application on a communication device of a user, where-in the set of features comprises:
      i. voice data;
      ii. textual data, wherein the textual data comprises text and character depicted expression;
      iii. location data;
      iv. application usage data;
      v. biometric data;
      vi. sleep data;
      vii. activity data; and
      viii. self-reported data;
   b. processing the set of features, using a neural network, to encode sentiment content from the set of features to determine a marker,
   c. wherein the neural network is configured to process missing features in the set of features,
   d. wherein the encoding discards semantic content from the set of features, and wherein the marker is predictive of the user's response to an intervention;
   e. determining an indication of a sentiment of the user based on the encoded sentiment content; and
   f. generating a treatment plan to the user based on the user's profile,
   g. wherein the profile comprises:
      i. the user's user preferences of the application,
      ii. the user's demographic information, and
      iii. the user's engagement with the application.
Embodiment 140: The method of Embodiment 139, wherein the voice data is processed by any combination of one or more neural networks.
Embodiment 141: The method of Embodiment 139 or 140, wherein the marker accounts for hormonal cycles.
Embodiment 142: The method of any one of Embodiments 139-141, wherein the biometric data further comprises changes arising from hormonal cycles.
Embodiment 143: The method of any one of Embodiments 139-142, wherein the intervention comprises anti-psychotic and/or mood-altering medication.
Embodiment 144: The method of any one of Embodiments 139-143, wherein the intervention comprises counseling and/or psychotherapy.
Embodiment 145: The method of any one of Embodiments 139-144, wherein the intervention comprises following sleep hygiene such as a sleep schedule.
Embodiment 146: The method of any one of Embodiments 139-145, wherein the user is stratified into a group based on the user's historical data, the historical data comprising history of trauma, adverse childhood experiences, family history, personal history, personal characteristics, or any combination thereof.
Embodiment 147: The method of any one of Embodiments 139-146, wherein the treatment plan is designed to improve an academic performance (including matriculation and/or retention) of the user.
Embodiment 148: A method of training a model for generate a treatment plan for a user, comprising:
   a. collecting a set of features from an application on a communication device of a user, wherein the set of features comprises:
      i. voice data;
      ii. textual data, wherein the textual data comprises text and character depicted expression;
      iii. location data;
      iv. application usage data;
      v. biometric data;
      vi. sleep data;
      vii. activity data; and
      viii. self-reported data;
   b. training a first neural network to encode sentiment content from the set of features to determine a marker,
      i. wherein the neural network is configured to process missing features in the set of features,
      ii. wherein the encoding discards semantic content from the set of features, and wherein the marker is predictive of the user's response to an intervention,
      iii. wherein the encoded sentiment content provides an indication of a sentiment of the user; and
   c. training a second neural network to generate a treatment plan to the user based on the user's profile,
   d. wherein the profile comprises:
      i. the user's user preferences of the application,
      ii. the user's demographic information, and
      iii. the user's engagement with the application.
Embodiment 149: A system for training a model to assess a mental health status of a user, the system comprising:
   a. one or more processors;
   b. a memory comprising executable instructions which, when executed by the one or more processors, cause the system to:
   c. collect marker values of a population of test users from two or more data channels, selected from the group consisting of passive data channels, active data channels, self-reported data channels, and external data channels;
   d. extract a set of features from the marker values; and
   e. train a model using the set of features, wherein the model assesses a mental health status based on the set of features.
Embodiment 150: A system to assess a mental health status of a user, the system comprising:
   a. one or more processors;
   b. a memory comprising executable instructions which, when executed by the one or more processors, cause the system to:
   c. collect marker values of the user from two or more data channels; and
   d. use a model trained pursuant to the system of Embodiment 141 to assess a mental health status of the user.
Embodiment 151: The system of Embodiment 150, wherein the one or more processors is configured to cause the system to continuously monitor the mental health status of the user.
Embodiment 152: The system of Embodiment 149, wherein the set of features comprises features from two or more of the data channels.
Embodiment 153: The system of Embodiment 149, wherein the set of features comprises features from three or more of the data channels.
Embodiment 154: The system of Embodiment 149, wherein the set of features comprises features from four of the data channels.
Embodiment 155: The system of any one of Embodiments 149, 150, 233, 235, 260, 271, 272, 273, or 286, wherein the marker values comprise values from a passive data channel.
Embodiment 156: The system of Embodiment 155, wherein the marker values from the passive data channel are selected from the group consisting of location data, wearables data, and device usage data.
Embodiment 157: The system of Embodiment 156, wherein the device usage data is collected by the user uploading a screenshot of a previous week's application usage on a smartphone.
Embodiment 158: The system of Embodiment 156, wherein the device usage data is collected automatically from a smartphone of the user.
Embodiment 159: The system of Embodiment 156, wherein the marker values from the passive data channel comprise location data selected from the group consisting of location, time spent at location, location type, and location frequency.
Embodiment 160: The system of Embodiment 159, wherein the location is selected from the group consisting of home, gym, school, restaurant, bar, church, and other.
Embodiment 161: The system of Embodiment 156, wherein the marker values from a passive data channel comprise wearables data selected from the group consisting of a user's heartrate, body temperature, activity, sleep, respirations, menstrual status, stress level, and combinations thereof.
Embodiment 162: The system of Embodiment 161, wherein the wearables data comprises activity data and is selected from the group consisting of steps taken, floors climbed, intensity minutes, calories burned, and combinations thereof.
Embodiment 163: The system of Embodiment 161, wherein the wearables data comprises sleep data and is selected from the group consisting of bedtime, wake up time, sleep duration, quality of sleep, and combinations thereof.
Embodiment 164: The system of Embodiment 156, wherein the marker values from a passive data channel comprise device usage data selected from the group consisting of app usage, battery usage and charging, call frequency and duration, location tracking data, mental health-related internet searches, overall screen time, category specific screen time, physical activity levels (e.g., step counts), sleep patterns inferred from phone activity, social media usage patterns, text message frequency, typing speed and pressure, usage of mental health apps, voice tone and pitch analysis during calls, and frequency and content changes in photos and videos.
Embodiment 165: The system of Embodiment 164, wherein category specific screen time is selected from the group consisting of social, entertainment, educational, and informational.
Embodiment 166: The system of Embodiment 164, wherein device usage data is encoded.
Embodiment 167: The system of Embodiment 166, wherein the device usage data is encoded by extracting sentiment and not semantic content.
Embodiment 168: The system of Embodiment 166, wherein the device usage data is encoded by a token to randomize said device usage data.
Embodiment 169: The system of Embodiment 156, wherein the device usage data comprises data derived from one or more screenshots.
Embodiment 170: The system of Embodiment 169, wherein the data derived from one or more screenshots comprises phone usage.
Embodiment 171: The system of Embodiment 170, wherein the one or more screenshots further comprises application usage on a phone.
Embodiment 172: The system of Embodiment 169, wherein the data derived from one or more screenshots comprises health data from a health tracking application.
Embodiment 173: The system of Embodiment 149 or 150, wherein the marker values comprise values from an active data channel.
Embodiment 174: The system of Embodiment 173, wherein the marker values from an active data channel comprise voice values data.
Embodiment 175: The system of Embodiment 174, wherein the voice values data is selected from the group consisting of voice characteristics, speech characteristics, background noise characteristics, and combinations thereof.
Embodiment 176: The system Embodiments 174, wherein the voice values data comprises passive noise data.
Embodiment 177: The system of Embodiment 174, wherein the voice values data is selected from the group consisting of tone of voice, inflection of voice, word count, speech rate, intensity of voice, pitch, magnitude, phonetics, tempo-spectral, formant, glottal closure instance and combinations thereof.
Embodiment 178: The system of Embodiment 149 or 150, wherein the marker values comprise values from a self-reported data channel.
Embodiment 179: The system of Embodiment 178, wherein the self-reported data channel comprises values from self-reported data.
Embodiment 180: The system of Embodiment 178, wherein the marker values from a self-reported data channel comprise data from a questionnaire.
Embodiment 181: The system of Embodiment 180, wherein the questionnaire is completed by a user, a user's supervisor, a user's co-worker, a user's teacher, a user's counselor, a user's family member, a user's friend, or a combination thereof.
Embodiment 182: The system of Embodiment 180, wherein the questionnaire comprises questions related to demographic, family history, health history, impairments, hobbies, mental health history, family mental health history, academic history, romantic history, exercise details, drug and alcohol use and history, sleep, diet, emotional status and history, socialization, recurrent thoughts, physical and biological signs, or a combination thereof.
Embodiment 183: The system of Embodiment 180, wherein the questionnaire comprises demographic questions selected from the group consisting of age, sex, gender identity, sexual orientation, race, ethnicity, religion, or any combination thereof.
Embodiment 184: The system of Embodiment 178, wherein the marker values from a self-reported data channel comprises an emotional identifier.
Embodiment 185: The system of Embodiment 178, wherein the marker values from a self-reported data channel comprises a daily emotional identifier.
Embodiment 186: The system of Embodiment 149 or 150, wherein the marker values comprise values from an external data channel.
Embodiment 187: The system of Embodiment 186, wherein the values from an external data channel are selected from the group consisting of weather reports, local current events, and global current events.
Embodiment 188: The system of Embodiment 149, wherein the features are selected using summary statistics.
Embodiment 189: The system of Embodiment 149, wherein the features are selected based on a latent space.
Embodiment 190: The system of Embodiment 189, wherein the latent space is based on a transformation of the marker values into the latent space.
Embodiment 191: The system of Embodiment 149, wherein each feature of the set of features selected improves the assessment of the mental health status.
Embodiment 192: The system of Embodiment 149, wherein an absence of a marker value is one of the set of features.
Embodiment 193: The system of Embodiment 149, wherein the model is trained using a machine learning algorithm selected from principal component analysis (PCA), uniform manifold approximation and projection (UMAP), artificial neural networks (e.g. variational autoencoder (VAE), recurrent neural networks (RNNs), long short-term memory networks (LSTMs), transformers), time series, and any combination thereof.
Embodiment 194: The system of Embodiment 150, wherein the one or more processors is configured to assess a change in the mental health status of the user.
Embodiment 195: The system of Embodiment 150, wherein the one or more processors is configured to assess a baseline mental health status of the user.
Embodiment 196: The system of Embodiment 150, wherein the one or more processors is configured to assess change in mental health status of the user relative to a baseline mental health status of the user.
Embodiment 197: The system of Embodiment 150, wherein the one or more processors is configured to predict a mental health trajectory of the user.
Embodiment 198: The system of Embodiment 150, wherein the one or more processors is configured to calculate a probability of a mental health status of the user.
Embodiment 199: The system of Embodiment 150, wherein the one or more processors is configured to refer the user to a mental health resource.
Embodiment 200: The system of Embodiment 199, wherein the mental health resource is selected based on a model.
Embodiment 201: The system of Embodiment 200, wherein the model comprises a machine learning model.
Embodiment 202: The system of Embodiment 201, wherein the model is trained based on data from users of the computer implemented system to assess a mental health status.
Embodiment 203: The system of any one of Embodiments 199-203, wherein the referring is done via a computing device or system.
Embodiment 204: The system of any one of Embodiments 199-203, wherein the mental health resource is delivered via a computing device or system.
Embodiment 205: The system of any one of Embodiments 199-203, wherein the mental health resource is selected based on a model that accounts for one or more of the following data types: mental health status of the user, sexual identity of the user, cultural background of the user, religious beliefs of the user, hobbies and interests of the user, location of the user, and combinations thereof.
Embodiment 206: The system of any one of Embodiments 199-203, wherein the one or more processors is configured to assess use by the user of the mental health resources.
Embodiment 207: The system of Embodiment 206, wherein the one or more processors is configured to assess time the user is at a location of the mental health resource.
Embodiment 208: The system of Embodiment 206, wherein the one or more processors is configured to assess time the user interacts with a website of the mental health resource.
Embodiment 209: The system of Embodiment 206, wherein the one or more processors is configured to assess data generated from an app used to provide the mental health resource.
Embodiment 210: The system of Embodiment 206, wherein the one or more processors is configured to assess changes in the mental health status of the user.
Embodiment 211: The system of Embodiment 206, wherein the one or more processors is configured to assess feedback from the user regarding the mental health resource.
Embodiment 212: The system of Embodiment 150, wherein the one or more processors is configured to report a clinical mental health status of the user.
Embodiment 213: The system of Embodiment 212, wherein the one or more processors is configured to report an anxiety or depression status of the user.
Embodiment 214: The system of Embodiment 212, wherein the one or more processors is configured to report a subclinical mental health status of the user.
Embodiment 215: The system of Embodiment 212, wherein the one or more processors is configured to report acceptance or loneliness.
Embodiment 216: The system of Embodiment 149, wherein the population of test users are students.
Embodiment 217: The system of Embodiment 150, wherein the user is a student.
Embodiment 218: The system of Embodiment 216, wherein the student is a college student.
Embodiment 219: The system of Embodiment 149, wherein the population of test users are 18 to 24 years of age.
Embodiment 220: The system of Embodiment 150, wherein the user is 18 to 24 years of age.
Embodiment 221: The system of Embodiment 150, wherein the one or more processors is configured to use the model to predict a matriculation status of the user.
Embodiment 222: A system for improving retention of students or employees, the system configured to perform the method of Embodiment 51, on a set of students or employees and thereby improving retention of students or employees of the set.
Embodiment 223: The system of Embodiment 149, wherein the one or more processors continuously collect marker values of the population of test users.
Embodiment 224: The system of Embodiment 150, wherein the one or more processors is configured to continuously collect marker values of the user.
Embodiment 225: The system of Embodiment 223 or 224, wherein the continuous markers are collected over 3 months.
Embodiment 226: The system of Embodiment 223 or 224, wherein the continuous markers are collected over 6 months.
Embodiment 227: The system of Embodiment 223 or 224, wherein the continuous markers are collected over 1 year.
Embodiment 228: The system of Embodiment 223 or 224, wherein the continuous markers are collected over a semester.
Embodiment 229: The system of Embodiment 223 or 224, wherein the continuous markers are collected over 2 semesters.
Embodiment 230: The system of any one of Embodiments 149-229, wherein the one or more processors is configured to encode the marker values from the data channels.
Embodiment 231: The system of Embodiment 230, wherein the one or more processors is configured to randomize the marker values from the data channels.
Embodiment 232: The system of Embodiment 230, wherein the one or more processors is configured to extract sentiment content and discard semantic content from the marker values from the data channels.
Embodiment 233: A system to assess a mental health status of a user, the system comprising:
   a. one or more processors;
   b. a memory comprising executable instructions which, when executed by the one or more processors, cause the system to:
   c. collect marker values of a population of test users, the marker values drawn from at least two of: passive data, active data, self-reported data, and external data; and
   d. extract a set of features from the marker values suitable for training a model.
Embodiment 234: The system of Embodiment 233, wherein the one or more processors is configured to generate a list of curated resources.
Embodiment 235: A system for training a model to assess a mental health status of a user, the system comprising:
   a. one or more processors;
   b. a memory comprising executable instructions which, when executed by the one or more processors, cause the system to:
   c. collect marker values of a population of test users, the marker values drawn from at least two of: passive data, active data, self-reported data, and external data; and
   d. train a model using the set of features, wherein the model assesses a mental health status based on the set of features.
Embodiment 236: The system of Embodiment 235, wherein the one or more processors generate a list of curated resources.
Embodiment 237: A system to extract a health conclusion from location data, the system comprising:
   a. one or more processors;
   b. a memory comprising executable instructions which, when executed by the one or more processors, cause the system to:
   c. collect location data on a user at one or more points in time;
   d. collect data on a local condition at the user's position(s); and
   e. use a model to draw a health conclusion based on the location data and local-conditions data.
Embodiment 238: The system of Embodiment 237 or 260, wherein the local conditions comprise weather, news, local events, or any combination thereof.
Embodiment 239: The system of Embodiment 237 or 260, wherein the model considers multiple local conditions.
Embodiment 240: The system of Embodiment 237 or 260, wherein the model considers more than one user.
Embodiment 241: The system of Embodiment 237 or 260, wherein the one or more processors is configured to generate a list of curated resources related to the health conclusion.
Embodiment 242: A system to extract a health conclusion from a user's voice, the system comprising:
   a. one or more processors;
   b. a memory comprising executable instructions which, when executed by the one or more processors, cause the system to:
      i. collect from a first instance of a user's voice at least one of: a vocal cord characteristic, a speech characteristic, and a background noise characteristic;
      ii. collect from a second instance of a user's voice at least one of: a vocal cord characteristic, a speech characteristic, and a background noise characteristic; and
      iii. use a model to draw a health conclusion based on characteristics collected from the first and second instances.
Embodiment 243: The system of Embodiment 242 or 261, wherein at least one of the first and second instances is recorded by the user.
Embodiment 244: The system of Embodiment 242 or 261, wherein at least one of the first and second instances is streamed by the user.
Embodiment 245: The system of Embodiment 242 or 261, wherein the one or more processors is configured to generate a list of curated resources related to the health conclusion.
Embodiment 246: The system of Embodiment 242, wherein the one or more processors is comprised in a smartphone.
Embodiment 247: The system of Embodiment 242 or 261, wherein a health conclusion comprises a mental health status.
Embodiment 248: The system of Embodiment 247, wherein a mental health status comprises any of healthy, depressive, anxious, and behavioral.
Embodiment 249: The system of Embodiment 242 or 261, wherein characteristics comprise at least one of tone of voice, inflection of voice, word count, speech rate, intensity of voice, pitch, magnitude, phonetics, tempo-spectral, formant, glottal closure instance, or any combinations thereof.
Embodiment 250: A system to extract a health conclusion from device usage data, the system comprising:
   a. one or more processors;
   b. a memory comprising executable instructions which, when executed by the one or more processors, cause the system to:
      i. collect a user's device usage data at one or more points in time; and
      ii. use a model to draw a health conclusion based on the device usage data.
Embodiment 251: The system of Embodiment 250 or 262, wherein the device usage data comprises a total of time a user spent on a device.
Embodiment 252: The system of Embodiment 250 or 262, wherein the device usage data comprises a total of time using one or more specific apps.
Embodiment 253: The system of Embodiment 250 or 262, wherein the device usage data comprises a total of time using one or more categories of apps.
Embodiment 254: The system of Embodiment 253 or 262, wherein the categories comprise any one of social, entertainment, educational, and informational.
Embodiment 255: A system to extract a health conclusion from a user's device, the system comprising:
   a. one or more processors;
   b. a memory comprising executable instructions which, when executed by the one or more processors, cause the system to:
      i. collect, from a device at multiple points in time, data on a user's positioning, voice, and device usage; and
      ii. use a model to draw a health conclusion based on the collected data.
Embodiment 256: A system to provide health information for a user, the system comprising:
   a. one or more processors;
   b. a memory comprising executable instructions which, when executed by the one or more processors, cause the system to:
      i. collect data about a person;
      ii. use a model to draw a health conclusion based on the collected data; and
      iii. provide at least one health resource option based on the health conclusion.
Embodiment 257: The system of Embodiment 256 or 264, wherein the data includes self-reported data.
Embodiment 258: The system of Embodiment 257, wherein the self-reported data includes private data.
Embodiment 259: The system of Embodiment 257, wherein the self-reported data includes encoded data.
Embodiment 260: A system for training a model to generate a health conclusion from location data, the system comprising:
   a. one or more processors;
   b. a memory comprising executable instructions which, when executed by the one or more processors, cause the system to:
      i. collect location data on a user at one or more points in time;
      ii. collect data on a local condition at the user's position(s);
      iii. extract marker values from the location data and local-conditions data; and
      iv. train a model to generate a health conclusion based on marker values.
Embodiment 261: A system for training a model to generate a health conclusion from a user's voice, the system comprising:
   a. one or more processors;
   b. a memory comprising executable instructions which, when executed by the one or more processors, cause the system to:
      i. collect from a first instance of a user's voice at least one of: a vocal cord characteristic, a speech characteristic, and a background noise characteristic;
      ii. collect from a second instance of a user's voice at least one of: a vocal cord characteristic, a speech characteristic, and a background noise characteristic; and
      iii. train a model to generate a health conclusion based on characteristics collected from the first and second recordings.
Embodiment 262: A system for training a model to generate a health conclusion from device usage data, the system comprising:
   a. one or more processors;
   b. a memory comprising executable instructions which, when executed by the one or more processors, cause the system to:
      i. collect a user's device usage data at one or more points in time; and
      ii. train a model to generate a health conclusion based on the device usage data.
Embodiment 263: A system for training a model to generate a health conclusion from a user's device, the system comprising:
   a. one or more processors;
   b. a memory comprising executable instructions which, when executed by the one or more processors, cause the system to:
      i. collect, from a device at multiple points in time, data on a user's positioning, voice, and device usage; and
      ii. train a model to generate a health conclusion based on the collected data.
Embodiment 264: The system of Embodiment 255 or 263, wherein the data on the device usage comprises an amount of time spent on the device.
Embodiment 265: The system of Embodiment 255 or 263, wherein the data on the device usage comprises an amount of time spent on one or more apps or categories thereof.
Embodiment 266: The system of Embodiment 255 or 263, wherein the data on the user's positioning comprises location data taken at multiple points in time.
Embodiment 267: The system of Embodiment 255 or 263, wherein the data on the user's positioning comprises a local condition at the user's position(s), such as weather, news, local events, or any combination thereof.
Embodiment 268: The system of Embodiment 255 or 263, wherein the data on the voice comprises voice comprises first and second instances of the user's voice.
Embodiment 269: The system of Embodiment 268, wherein the first and second instances comprise at least one of a vocal cord characteristic, a speech characteristic, and a background noise characteristic.
Embodiment 270: A system for training a model to generate health information for a user, the system comprising:
   a. one or more processors;
   b. a memory comprising executable instructions which, when executed by the one or more processors, cause the system to:
      i. collect data about a user; and
      ii. train a model to generate health information based on the collected data.
Embodiment 271: A system for training a model to assess a mental health status of a user, the system comprising:
   a. one or more processors;
   b. a memory comprising executable instructions which, when executed by the one or more processors, cause the system to:
      i. collect marker values of a population of test users, the marker values drawn from at least two of: passive data, active data, self-reported data, and external data;
      ii. extract a set of features from the marker values; and
      iii. train a model using the set of features, wherein the model assesses a mental health status based on the set of features.
Embodiment 272: A system for training a model to assess a performance outcome of a user, the system comprising:
   a. one or more processors;
   b. a memory comprising executable instructions which, when executed by the one or more processors, cause the system to:
      i. collect marker values of a population of test users, the marker values drawn from at least two of: passive data, active data, self-reported data, and external data;
      ii. extract a set of features from the marker values; and
      iii. train a model using the set of features, wherein the model assesses a mental health status based on the set of features.
Embodiment 273: A system for assessing a performance outcome of a user, the system comprising:
   a. one or more processors;
   b. a memory comprising executable instructions which, when executed by the one or more processors, cause the system to:
      i. collect marker values of a user, the marker values drawn from at least two of: passive data, active data, self-reported data, and external data;
      ii. extract a set of features from the marker values; and
      iii. predict, using a model, a performance outcome of the user based on the set of features.
Embodiment 274: The system of Embodiment 272 or 273, wherein the performance outcome comprises attrition, grades, changes in major, taking longer to graduate, retention, or academic performance.
Embodiment 275: The system of any one of Embodiments 1-274, wherein the mental health condition comprises any one of depression, anxiety, and behavior.
Embodiment 276: The system of Embodiment 275, wherein the behavior comprises substance use and/or substance abuse.
Embodiment 277: The system of any one of Embodiments 1-276, wherein training the model comprises more than one user.
Embodiment 278: The system of any one of Embodiments 1-277, wherein the model considers some specific combination of features.
Embodiment 279: The system of any one of Embodiments 1-278, wherein the model considers changes in device usage data over time.
Embodiment 280: The system of any one of Embodiments 1-279, wherein the health conclusions comprise depression, anxiety, and/or behavior.
Embodiment 281: The system of any one of Embodiments 1-280, wherein the one or more processors is configured to generate a list of curated resources related to the health conclusion.
Embodiment 282: The system of any one of Embodiments 1-281, wherein the one or more processors is configured to identify curated resources related to the health conclusion.
Embodiment 283: The system of any one of Embodiments 1-282, wherein the one or more processors is configured to use a predefined table and/or dataset that matches resource options to health conclusions.
Embodiment 284: The system of any one of Embodiments 1-283, wherein the one or more processors is configured to select the resource option(s) to provide based on a ranking of available options.
Embodiment 285: The system of any one of Embodiments 1-284, wherein the one or more processors is configured to update the data collection, generating an updated health conclusion, and providing an updated resource option.
Embodiment 286: A system to assess a mental health status of a user, the system comprising:
   a. one or more processors;
   b. a memory comprising executable instructions which, when executed by the one or more processors, cause the system to:
      i. collect marker values of the user from two or more data channels;
      ii. extract a set of features from the marker values;
      iii. train a model using the set of features, wherein the model assesses a mental health status based on the set of features; and
      iv. use the model to assess a mental health status of the user.
Embodiment 287: A system to generate a treatment plan to a user, the system comprising:
   a. one or more processors;
   b. a memory comprising executable instructions which, when executed by the one or more processors, cause the system to:
      i. collect a set of features from an application on a communication device of a user, where-in the set of features comprises:
         1. voice data;
         2. textual data, wherein the textual data comprises text and character depicted expression;
         3. location data;
         4. application usage data;
         5. biometric data;
         6. sleep data;
         7. activity data; and
         8. self-reported data;
      ii. process the set of features, using a neural network, to encode sentiment content from the set of features to determine a marker, wherein the neural network is configured to process missing features in the set of features, wherein the encoding discards semantic content from the set of features, and wherein the marker is predictive of the user's response to an intervention;
      iii. determine an indication of a sentiment of the user based on the encoded sentiment content; and
      iv. generate a treatment plan to the user based on the user's profile, wherein the profile comprises:
         1. the user's user preferences of the application,
         2. the user's demographic information, and
         3. the user's engagement with the application.
Embodiment 288: The system of Embodiment 287, wherein the voice data is processed by an artificial neural network (e.g. autoregressive neural network, a recurrent neural network, a LSTM neural network, a large language model, and a transformer).
Embodiment 289: The system of Embodiment 287 or 288, wherein the marker accounts for hormonal cycles.
Embodiment 290: The system of any one of Embodiments 287-289, wherein the biometric data further comprises changes arising from hormonal cycles.
Embodiment 291: The system of any one of Embodiments 287-290, wherein the intervention comprises anti-psychotic and/or mood-altering medication.
Embodiment 292: The system of any one of Embodiments 287-291, wherein the intervention comprises counseling and/or psychotherapy.
Embodiment 293: The system of any one of Embodiments 287-292, wherein the intervention comprises following sleep hygiene such as a sleep schedule.
Embodiment 294: The system of any one of Embodiments 287-293, wherein the user is stratified into a group based on the user's historical data, the historical data comprising history of trauma, adverse childhood experiences, family history, personal history, personal characteristics, or any combination thereof.
Embodiment 295: The system of any one of Embodiments 287-294, wherein the treatment plan is designed to improve an academic performance (including matriculation and/or retention) of the user.
Embodiment 296: A system to generate a treatment plan to a user, the system comprising:
   a. one or more processors;
   b. a memory comprising executable instructions which, when executed by the one or more processors, cause the system to:
      i. collect a set of features from an application on a communication device of a user, wherein the set of features comprises:
         1. voice data;
         2. textual data, wherein the textual data comprises text and character depicted expression;
         3. location data;
         4. application usage data;
         5. biometric data;
         6. sleep data;
         7. activity data; and
         8. self-reported data;
      ii. train a neural network to encode sentiment content from the set of features to determine a marker, wherein the neural network is configured to process missing features in the set of features, wherein the encoding discards semantic content from the set of features, wherein the marker is predictive of the user's response to an intervention, and wherein the encoded sentiment content is indicative of a sentiment of the user;
      iii. train a second neural network to generate a treatment plan to the user based on the user's profile, wherein the profile comprises: 1. the user's user preferences of the application, 2. the user's demographic information, and 3. the user's engagement with the application.
Embodiment 297: A non-transitory, computer-readable medium with computer-readable instructions for training a model for assessing a mental health status or a change thereof of a user, the instructions comprising instructions for:
   a. collecting marker values of a population of test users from two or more data channels, selected from the group consisting of passive data channels, active data channels, self-reported data channels, and external data channels;
   b. extracting a set of features from the marker values; and
   c. training a model using the set of features, wherein the model assesses a mental health status based on the set of features.
Embodiment 298: A non-transitory, computer-readable medium with computer-readable instructions for training a model for assessing a mental health status or a change thereof of a user, the instructions comprising instructions for:
   a. collecting marker values of the user from two or more data channels; and
   b. using a model trained pursuant to the method of Embodiment 281 to assess a mental health status of the user.
Embodiment 299: The non-transitory, computer-readable medium of Embodiment 298, wherein the assessing comprises ongoing monitoring of the mental health status of the user.
Embodiment 300: The non-transitory, computer-readable medium of Embodiment 297, wherein the set of features comprises features from two or more of the data channels.
Embodiment 301: The non-transitory, computer-readable medium of Embodiment 297, wherein the set of features comprises features from three or more of the data channels.
Embodiment 302: The non-transitory, computer-readable medium of Embodiment 297, wherein the set of features comprises features from four of the data channels.
Embodiment 303: The non-transitory, computer-readable medium of any one of Embodiments 297, 298, 381, 383, 408, 419-421, and 434, wherein the marker values comprise values from a passive data channel.
Embodiment 304: The non-transitory, computer-readable medium of Embodiment 303, wherein the marker values from the passive data channel are selected from the group consisting of location data, wearables data, and device usage data.
Embodiment 305: The non-transitory, computer-readable medium of Embodiment 304, wherein the device usage data is collected by the user uploading a screenshot of a previous week's application usage on a smartphone.
Embodiment 306: The non-transitory, computer-readable medium of Embodiment 304, wherein the device usage data is collected automatically from a smartphone of the user.
Embodiment 307: The non-transitory, computer-readable medium of Embodiment 304, wherein the marker values from the passive data channel comprise location data selected from the group consisting of location, time spent at location, location type, and location frequency.
Embodiment 308: The non-transitory, computer-readable medium of Embodiment 307, wherein the location is selected from the group consisting of home, gym, school, restaurant, bar, church, and other.
Embodiment 309: The non-transitory, computer-readable medium of Embodiment 304, wherein the marker values from a passive data channel comprise wearables data selected from the group consisting of a user's heartrate, body temperature, activity, sleep, respirations, menstrual status, stress level, and combinations thereof.
Embodiment 310: The non-transitory, computer-readable medium of Embodiment 309, wherein the wearables data comprises activity data and is selected from the group consisting of steps taken, floors climbed, intensity minutes, calories burned, and combinations thereof.
Embodiment 311: The non-transitory, computer-readable medium of Embodiment 309, wherein the wearables data comprises sleep data and is selected from the group consisting of bedtime, wake up time, sleep duration, quality of sleep, and combinations thereof.
Embodiment 312: The non-transitory, computer-readable medium of Embodiment 304, wherein the marker values from a passive data channel comprise device usage data selected from the group consisting of app usage, battery usage and charging, call frequency and duration, location tracking data, mental health-related internet searches, overall screen time, category specific screen time, physical activity levels (e.g., step counts), sleep patterns inferred from phone activity, social media usage patterns, text message frequency, typing speed and pressure, usage of mental health apps, voice tone and pitch analysis during calls, and frequency and content changes in photos and videos.
Embodiment 313: The non-transitory, computer-readable medium of Embodiment 312, wherein category specific screen time is selected from the group consisting of social, entertainment, educational, and informational.
Embodiment 314: The non-transitory, computer-readable medium of Embodiment 312, wherein device usage data is encoded.
Embodiment 315: The non-transitory, computer-readable medium of Embodiment 314, wherein the device usage data is encoded by extracting sentiment and not semantic content.
Embodiment 316: The non-transitory, computer-readable medium of Embodiment 314, wherein the device usage data is encoded by a token to randomize said device usage data.
Embodiment 317: The non-transitory, computer-readable medium of Embodiment 310, wherein the device usage data comprises data derived from one or more screenshots.
Embodiment 318: The non-transitory, computer-readable medium of Embodiment 317, wherein the data derived from one or more screenshots comprises phone usage.
Embodiment 319: The non-transitory, computer-readable medium of Embodiment 318, wherein the one or more screenshots further comprises application usage on a phone.
Embodiment 320: The non-transitory, computer-readable medium of Embodiment 317, wherein the data derived from one or more screenshots comprises health data from a health tracking application.
Embodiment 321: The non-transitory, computer-readable medium of Embodiment 297 or 298, wherein the marker values comprise values from an active data channel.
Embodiment 322: The non-transitory, computer-readable medium of Embodiment 321, wherein the marker values from an active data channel comprise voice values data.
Embodiment 323: The non-transitory, computer-readable medium of Embodiment 322, wherein the voice values data is selected from the group consisting of voice characteristics, speech characteristics, background noise characteristics, and combinations thereof.
Embodiment 324: The non-transitory, computer-readable medium Embodiments 322, wherein the voice values data comprises passive noise data.
Embodiment 325: The non-transitory, computer-readable medium of Embodiment 322, wherein the voice values data is selected from the group consisting of tone of voice, inflection of voice, word count, speech rate, intensity of voice, pitch, magnitude, phonetics, tempo-spectral, formant, glottal closure instance and combinations thereof.
Embodiment 326: The non-transitory, computer-readable medium of Embodiment 297 or 298, wherein the marker values comprise values from a self-reported data channel.
Embodiment 327: The non-transitory, computer-readable medium of Embodiment 326, wherein the self-reported data channel comprises values from self-reported data.
Embodiment 328: The non-transitory, computer-readable medium of Embodiment 326, wherein the marker values from a self-reported data channel comprise data from a questionnaire.
Embodiment 329: The non-transitory, computer-readable medium of Embodiment 328, wherein the questionnaire is completed by a user, a user's supervisor, a user's co-worker, a user's teacher, a user's counselor, a user's family member, a user's friend, or a combination thereof.
Embodiment 330: The non-transitory, computer-readable medium of Embodiment 328, wherein the questionnaire comprises questions related to demographic, family history, health history, impairments, hobbies, mental health history, family mental health history, academic history, romantic history, exercise details, drug and alcohol use and history, sleep, diet, emotional status and history, socialization, recurrent thoughts, physical and biological signs, or a combination thereof.
Embodiment 331: The non-transitory, computer-readable medium of Embodiment 328, wherein the questionnaire comprises demographic questions selected from the group consisting of age, sex, gender identity, sexual orientation, race, ethnicity, religion, or any combination thereof.
Embodiment 332: The non-transitory, computer-readable medium of Embodiment 326, wherein the marker values from a self-reported data channel comprises an emotional identifier.
Embodiment 333: The non-transitory, computer-readable medium of Embodiment 326, wherein the marker values from a self-reported data channel comprises a daily emotional identifier.
Embodiment 334: The non-transitory, computer-readable medium of Embodiment 297 or 298, wherein the marker values comprise values from an external data channel.
Embodiment 335: The non-transitory, computer-readable medium of Embodiment 334, wherein the values from an external data channel are selected from the group consisting of weather reports, local current events, and global current events.
Embodiment 336: The non-transitory, computer-readable medium of Embodiment 297, wherein the features are selected using summary statistics.
Embodiment 337: The non-transitory, computer-readable medium of Embodiment 297, wherein the features are selected based on a latent space.
Embodiment 338: The non-transitory, computer-readable medium of Embodiment 337, wherein the latent space is based on a transformation of the marker values into the latent space.
Embodiment 339: The non-transitory, computer-readable medium of Embodiment 297, wherein each feature of the set of features selected improves the assessment of the mental health status.
Embodiment 340: The non-transitory, computer-readable medium of Embodiment 297, wherein an absence of a marker value is one of the set of features.
Embodiment 341: The non-transitory, computer-readable medium of Embodiment 297, wherein the model is trained using a machine learning algorithm selected from principal component analysis (PCA), uniform manifold approximation and projection (UMAP), artificial neural network (e.g. variational autoencoder (VAE), recurrent neural networks (RNNs), long short-term memory networks (LSTMs), transformers), time series, and any combination thereof.
Embodiment 342: The non-transitory, computer-readable medium of Embodiment 298, wherein assessing a mental health status or a change thereof comprises assessing a change in mental health status of the user.
Embodiment 343: The non-transitory, computer-readable medium of Embodiment 298, wherein assessing a mental health status or a change thereof comprises assessing a baseline mental health status of the user.
Embodiment 344: The non-transitory, computer-readable medium of Embodiment 298, wherein assessing a mental health status or a change thereof comprises assessing a change in mental health status of the user relative to a baseline mental health status of the user.
Embodiment 345: The non-transitory, computer-readable medium of Embodiment 298, wherein assessing a mental health status or a change thereof comprises predicting a mental health trajectory of the user.
Embodiment 346: The non-transitory, computer-readable medium of Embodiment 298, wherein assessing a mental health status or a change thereof comprises calculating a probability of a mental health status of the user.
Embodiment 347: The non-transitory, computer-readable medium of Embodiment 298, comprising referring the user to a mental health resource.
Embodiment 348: The non-transitory, computer-readable medium of Embodiment 347, wherein the mental health resource is selected based on a model.
Embodiment 349: The non-transitory, computer-readable medium of Embodiment 348, wherein the model comprises a machine learning model.
Embodiment 350: The non-transitory, computer-readable medium of Embodiment 349, wherein the model is trained based on data from users of the computer implemented method of assessing a mental health status or a change thereof.
Embodiment 351: The non-transitory, computer-readable medium of any of Embodiments 347-350, wherein the referring is done via a computing device or system.
Embodiment 352: The non-transitory, computer-readable medium of any of Embodiments 347-350, wherein the mental health resource is delivered via a computing device or system.
Embodiment 353: The non-transitory, computer-readable medium of any of Embodiments 347-350, wherein the mental health resource is selected based on a model that accounts for one or more of the following data types: mental health status of the user, sexual identity of the user, cultural background of the user, religious beliefs of the user, hobbies and interests of the user, location of the user, and combinations thereof.
Embodiment 354: The non-transitory, computer-readable medium of any of Embodiments 347-350, the method further comprising assessing use by the user of the mental health resources.
Embodiment 355: The non-transitory, computer-readable medium of Embodiment 354, wherein the assessing use by the user comprises assessing time the user is at a location of the mental health resource.
Embodiment 356: The non-transitory, computer-readable medium of Embodiment 354, wherein the assessing use by the user comprises assessing time the user interacts with a website of the mental health resource.
Embodiment 357: The non-transitory, computer-readable medium of Embodiment 354, wherein the assessing use by the user comprises assessing data generated from an app used to provide the mental health resource.
Embodiment 358: The non-transitory, computer-readable medium of Embodiment 354, wherein the assessing use by the user comprises assessing changes in the mental health status of the user.
Embodiment 359: The non-transitory, computer-readable medium of Embodiment 354, wherein the assessing use by the user comprises assessing feedback from the user regarding the mental health resource.
Embodiment 360: The non-transitory, computer-readable medium of Embodiment 298, the method further comprising reporting a clinical mental health status of the user.
Embodiment 361: The non-transitory, computer-readable medium of Embodiment 360, wherein reporting a clinical mental health status of the user comprises reporting an anxiety or depression status of the user.
Embodiment 362: The non-transitory, computer-readable medium of Embodiment 360, wherein reporting a clinical mental health status of the user comprises reporting a subclinical mental health status of the user.
Embodiment 363: The non-transitory, computer-readable medium of Embodiment 360, wherein reporting a clinical mental health status of the user comprises reporting acceptance or loneliness.
Embodiment 364: The non-transitory, computer-readable medium of Embodiment 297, wherein the population of test users are students.
Embodiment 365: The non-transitory, computer-readable medium of Embodiment 298, wherein the user is a student.
Embodiment 366: The non-transitory, computer-readable medium of Embodiment 364, wherein the student is a college student.
Embodiment 367: The non-transitory, computer-readable medium of Embodiment 297, wherein the population of test users are 18 to 24 years of age.
Embodiment 368: The non-transitory, computer-readable medium of Embodiment 298, wherein the user is 18 to 24 years of age.
Embodiment 369: The non-transitory, computer-readable medium of Embodiment 298, wherein the method further comprises using the model to predict a matriculation status of the user.
Embodiment 370: A non-transitory, computer-readable medium for improving retention of students or employees, comprising instructions for performing the method of Embodiment 51, on a set of students or employees and thereby improving retention of students or employees of the set.
Embodiment 371: The non-transitory, computer-readable medium of Embodiment 298, comprising continuously collecting marker values of the population of test users.
Embodiment 372: The non-transitory, computer-readable medium of Embodiment 281, comprising continuously collecting marker values of the user.
Embodiment 373: The non-transitory, computer-readable medium of Embodiment 371 or 372, wherein the continuous markers are collected over 3 months.
Embodiment 374: The non-transitory, computer-readable medium of Embodiment 371 or 372, wherein the continuous markers are collected over 6 months.
Embodiment 375: The non-transitory, computer-readable medium of Embodiment 371 or 372, wherein the continuous markers are collected over 1 year.
Embodiment 376: The non-transitory, computer-readable medium of Embodiment 371 or 372, wherein the continuous markers are collected over a semester.
Embodiment 377: The non-transitory, computer-readable medium of Embodiment 371 or 372, wherein the continuous markers are collected over 2 semesters.
Embodiment 378: The non-transitory, computer-readable medium of Embodiments 1-377, wherein the method further comprises encoding the marker values from the data channels.
Embodiment 379: The non-transitory, computer-readable medium of Embodiment 378, wherein the encoding comprises randomization of the marker values from the data channels.
Embodiment 380: The non-transitory, computer-readable medium of Embodiment 378, wherein the encoding comprises extracting sentiment content and discarding semantic content from the marker values from the data channels.
Embodiment 381: A non-transitory, computer-readable medium for assessing a mental health status or a change thereof of a user, comprising computer executable instructions for:
   a. collecting marker values of a population of test users, the marker values drawn from at least two of: passive data, active data, self-reported data, and external data; and
   b. extracting a set of features from the marker values suitable for training a model.
Embodiment 382: The non-transitory, computer-readable medium of Embodiment 381, the method further comprising generating a list of curated resources.
Embodiment 383: A non-transitory, computer-readable medium for training a model for assessing a mental health status or a change thereof of a user, comprising computer executable instructions for:
   a. collecting marker values of a population of test users, the marker values drawn from at least two of: passive data, active data, self-reported data, and external data; and
   b. training a model using the set of features, wherein the model assesses a mental health status based on the set of features.
Embodiment 384: The non-transitory, computer-readable medium of Embodiment 383, the method further comprising generating a list of curated resources.
Embodiment 385: A non-transitory, computer-readable medium for extracting a health conclusion from location data, comprising computer executable instructions for:
   a. collecting location data on a user at one or more points in time;
   b. collecting data on a local condition at the user's position(s); and
   c. using a model to draw a health conclusion based on the location data and local-conditions data.
Embodiment 386: The non-transitory, computer-readable medium of Embodiment 385 or 408, wherein the local conditions comprise weather, news, local events, or any combination thereof.
Embodiment 387: The non-transitory, computer-readable medium of Embodiment 385 or 408, wherein the model considers multiple local conditions.
Embodiment 388: The non-transitory, computer-readable medium of Embodiment 385 or 408, wherein the model considers more than one user.
Embodiment 389: The non-transitory, computer-readable medium of Embodiment 385 or 408, the method further comprising generating a list of curated resources related to the health conclusion.
Embodiment 390: A non-transitory, computer-readable medium for extracting a health conclusion from a user's voice, comprising computer executable instructions for:
   a. collecting from a first instance of a user's voice at least one of: a vocal cord characteristic, a speech characteristic, and a background noise characteristic;
   b. collecting from a second instance of a user's voice at least one of: a vocal cord characteristic, a speech characteristic, and a background noise characteristic; and
   c. using a model to draw a health conclusion based on characteristics collected from the first and second instances.
Embodiment 391: The non-transitory, computer-readable medium of Embodiment 390 or 409, wherein at least one of the first and second instances is recorded by the user.
Embodiment 392: The non-transitory, computer-readable medium of Embodiment 390 or 409, wherein at least one of the first and second instances is streamed by the user.
Embodiment 393: The non-transitory, computer-readable medium of Embodiment 390 or 409, the method further comprising generating a list of curated resources related to the health conclusion.
Embodiment 394: The non-transitory, computer-readable medium of Embodiment 390, wherein the computer executable instructions are comprised in a smartphone application.
Embodiment 395: The non-transitory, computer-readable medium of Embodiment 390 or 409, wherein a health conclusion comprises a mental health status.
Embodiment 396: The non-transitory, computer-readable medium of Embodiment 395, wherein a mental health status comprises any of healthy, depressive, anxious, and behavioral.
Embodiment 397: The non-transitory, computer-readable medium of Embodiment 390 or 409, wherein the characteristics comprise at least one of tone of voice, inflection of voice, word count, speech rate, intensity of voice, pitch, magnitude, phonetics, tempo-spectral, formant, glottal closure instance, or any combinations thereof.
Embodiment 398: A non-transitory, computer-readable medium for extracting a health conclusion from device usage data, comprising computer executable instructions for:
   a. collecting a user's device usage data at one or more points in time; and
   b. using a model to draw a health conclusion based on the device usage data.
Embodiment 399: The non-transitory, computer-readable medium of Embodiment 398 or 410, wherein the device usage data comprises a total of time a user spent on a device.
Embodiment 400: The non-transitory, computer-readable medium of Embodiment 398 or 410 wherein the device usage data comprises a total of time using one or more specific apps.
Embodiment 401: The non-transitory, computer-readable medium of Embodiment 398 or 410, wherein the device usage data comprises a total of time using one or more categories of apps.
Embodiment 402: The non-transitory, computer-readable medium of Embodiment 401, wherein the categories comprise any one of social, entertainment, educational, and informational.
Embodiment 403: A non-transitory, computer-readable medium for extracting a health conclusion from a user's device, comprising computer executable instructions for:
   a. collecting, from a device at multiple points in time, data on a user's positioning, voice, and device usage; and
   b. using a model to draw a health conclusion based on the collected data.
Embodiment 404: A non-transitory, computer-readable medium for providing health information for a user, comprising computer executable instructions for:
   a. collecting data about a person;
   b. using a model to draw a health conclusion based on the collected data; and
   c. providing at least one health resource option based on the health conclusion.
Embodiment 405: The non-transitory, computer-readable medium of Embodiment 404 or 418, wherein the data includes self-reported data.
Embodiment 406: The non-transitory, computer-readable medium of Embodiment 405, wherein the self-reported data includes private data.
Embodiment 407: The non-transitory, computer-readable medium of Embodiment 405, wherein the self-reported data includes encoded data.
Embodiment 408: A non-transitory, computer-readable medium for training a model for generating a health conclusion from location data, comprising computer executable instructions for:
   a. collecting location data on a user at one or more points in time;
   b. collecting data on a local condition at the user's position(s);
   c. extracting marker values from the location data and local-conditions data; and
   d. training a model to generate a health conclusion based on marker values.
Embodiment 409: A non-transitory, computer-readable medium for training a model to generate a health conclusion from a user's voice, comprising computer executable instructions for:
   a. collecting from a first instance of a user's voice at least one of: a vocal cord characteristic, a speech characteristic, and a background noise characteristic;
   b. collecting from a second instance of a user's voice at least one of: a vocal cord characteristic, a speech characteristic, and a background noise characteristic; and
   c. training a model to generate a health conclusion based on characteristics collected from the first and second recordings.
Embodiment 410: A non-transitory, computer-readable medium for training a model to generate a health conclusion from device usage data, comprising computer executable instructions for:
   a. collecting a user's device usage data at one or more points in time; and
   b. training a model to generate a health conclusion based on the device usage data.
Embodiment 411: A non-transitory, computer-readable medium for training a model to generate a health conclusion from a user's device, comprising computer executable instructions for:
   a. collecting, from a device at multiple points in time, data on a user's positioning, voice, and device usage; and
   b. training a model to generate a health conclusion based on the collected data.
Embodiment 412: The non-transitory, computer-readable medium of Embodiment 410 or 411, wherein the data on the device usage comprises an amount of time spent on the device.
Embodiment 413: The non-transitory, computer-readable medium of Embodiment 410 or 411, wherein the data on the device usage comprises an amount of time spent on one or more apps or categories thereof.
Embodiment 414: The non-transitory, computer-readable medium of Embodiment 410 or 411, wherein the data on the user's positioning comprises location data taken at multiple points in time.
Embodiment 415: The non-transitory, computer-readable medium of Embodiment 410 or 411, wherein the data on the user's positioning comprises a local condition at the user's position(s), such as weather, news, local events, or any combination thereof.
Embodiment 416: The non-transitory, computer-readable medium of Embodiment 410 or 411, wherein the data on the voice comprises voice comprises first and second instances of the user's voice.
Embodiment 417: The non-transitory, computer-readable medium of Embodiment 416, wherein the first and second instances comprise at least one of a vocal cord characteristic, a speech characteristic, and a background noise characteristic.
Embodiment 418: A non-transitory, computer-readable medium for training a model to generate health information for a user, comprising computer executable instructions for:
   a. collecting data about a user; and
   b. training a model to generate health information based on the collected data.
Embodiment 419: A non-transitory, computer-readable medium for training a model for assessing a mental health status or a change thereof of a user, comprising computer executable instructions for:
   a. collecting marker values of a population of test users, the marker values from at least two of: passive data, active data, self-reported data, and external data;
   b. extracting a set of features from the marker values; and
   c. training a model using the set of features, wherein the model assesses a mental health status based on the set of features.
Embodiment 420: A non-transitory, computer-readable medium for training a model for assessing a performance outcome of a user, comprising computer executable instructions for:
   a. collecting marker values of a population of test users, the marker values drawn from at least two of: passive data, active data, self-reported data, and external data;
   b. extracting a set of features from the marker values; and
   c. training a model using the set of features, wherein the model assesses a mental health status based on the set of features.
Embodiment 421: A non-transitory, computer-readable medium for assessing a performance outcome of a user, comprising computer executable instructions for:
   a. collecting marker values of a user, the marker values drawn from at least two of: passive data, active data, self-reported data, and external data;
   b. extracting a set of features from the marker values; and
   c. predicting, using a model, the performance outcome of the user based on the set of features.
Embodiment 422: The non-transitory, computer-readable medium of Embodiment 420 or 421, wherein the performance outcome comprises attrition, grades, changes in major, taking longer to graduate, retention, or academic performance.
Embodiment 423: The non-transitory, computer-readable medium of any one of Embodiments 1-422, wherein the mental health condition comprises any one of depression, anxiety, and behavior.
Embodiment 424: The non-transitory, computer-readable medium of Embodiment 423, wherein the behavior comprises substance use and/or substance abuse.
Embodiment 425: The non-transitory, computer-readable medium of any one of Embodiments 1-424, wherein training the model comprises more than one user.
Embodiment 426: The non-transitory, computer-readable medium of any one of Embodiments 1-425, wherein the model considers some specific combination of features.
Embodiment 427: The non-transitory, computer-readable medium of any one of Embodiments 1-426, wherein the model considers changes in device usage data over time.
Embodiment 428: The non-transitory, computer-readable medium of any one of Embodiments 1-427, wherein the health conclusions comprise depression, anxiety, and/or behavior.
Embodiment 429: The non-transitory, computer-readable medium of any one of Embodiments 1-428, the method further comprising generating a list of curated resources related to the health conclusion.
Embodiment 430: The non-transitory, computer-readable medium of any one of Embodiments 1-429, the method further comprising identifying curated resources related to the health conclusion.
Embodiment 431: The non-transitory, computer-readable medium of any one of Embodiments 1-430, the method further comprising using a predefined table and/or dataset that matches resource options to health conclusions.
Embodiment 432: The non-transitory, computer-readable medium of any one of Embodiments 1-431, the method further comprising selecting the resource option(s) to provide based on a ranking of available options.
Embodiment 433: The non-transitory, computer-readable medium of any one of Embodiments 1-432, the method further comprising updating the data collection, generating an updated health conclusion, and providing an updated resource option.
Embodiment 434: A non-transitory, computer-readable medium for assessing a mental health status or a change thereof of a user, comprising computer executable instructions for:
   a. collecting marker values of the user from two or more data channels;
   b. extracting a set of features from the marker values;
   c. training a model using the set of features, wherein the model assesses a mental health status based on the set of features; and
   d. using the model trained pursuant to the method of Embodiment Ito assess a mental health status of the user.
Embodiment 435: A non-transitory, computer-readable medium for generating a treatment plan to a user, comprising computer executable instructions for:
   a. collecting a set of features from an application on a communication device of a user, where-in the set of features comprises:
      i. voice data;
      ii. textual data;
      iii. location data;
      iv. application usage data;
      v. biometric data;
      vi. sleep data;
      vii. activity data; and
      viii. self-reported data;
   b. processing the set of features, using a neural network, to encode sentiment content from the set of features to determine a marker,
   c. wherein the neural network is configured to process missing features in the set of features,
   d. wherein the encoding discards semantic content from the set of features, and wherein the marker is predictive of the user's response to an intervention;
   e. determining an indication of a sentiment of the user based on the encoded sentiment content; and
   f. generating a treatment plan to the user based on the user's profile,
   g. wherein the profile comprises:
      i. the user's user preferences of the application,
      ii. the user's demographic information, and
      iii. the user's engagement with the application.
Embodiment 436: The non-transitory, computer-readable medium of Embodiment 434 or 435, wherein the voice data is processed by an artificial neural network (e.g. autoregressive neural network, a recurrent neural network, a LSTM neural network, a large language model, and a transformer).
Embodiment 437: The non-transitory, computer-readable medium of Embodiment 434 or 435, wherein the marker accounts for hormonal cycles.
Embodiment 438: The non-transitory, computer-readable medium of Embodiment 434 or 435, wherein the biometric data further comprises changes arising from hormonal cycles.
Embodiment 439: The non-transitory, computer-readable medium of Embodiment 434 or 435, wherein the intervention comprises anti-psychotic or mood-altering medication.
Embodiment 440: The non-transitory, computer-readable medium of Embodiment 434 or 435, wherein the intervention comprises counseling and/or psychotherapy.
Embodiment 441: The non-transitory, computer-readable medium of Embodiment 434 or 435, wherein the intervention comprises following sleep hygiene such as a sleep schedule.
Embodiment 442: The non-transitory, computer-readable medium of Embodiment 434 or 435, wherein the user is stratified into a group based on the user's historical data, the historical data comprising history of trauma, adverse childhood experiences, family history, personal history, personal characteristics, or any combination thereof.
Embodiment 443: The non-transitory, computer-readable medium of Embodiment 434 or 435, wherein the treatment plan is designed to improve an academic performance (including matriculation and/or retention) of the user.
Embodiment 444: A non-transitory, computer-readable medium for generating a treatment plan to a user, comprising computer executable instructions for:
   a. collecting a set of features from an application on a communication device of a user, where-in the set of features comprises:
      i. voice data;
      ii. textual data;
      iii. location data;
      iv. application usage data;
      v. biometric data;
      vi. sleep data;
      vii. activity data; and
      viii. self-reported data;
   b. train a neural network to encode sentiment content from the set of features to determine a marker,
      i. wherein the neural network is configured to process missing features in the set of features,
      ii. wherein the encoding discards semantic content from the set of features, and wherein the marker is predictive of the user's response to an intervention, and
      iii. wherein the encoded sentiment content is indicative of a sentiment of the user;
   c. train a second neural network to generate a treatment plan to the user based on the user's profile, wherein the profile comprises:
      i. the user's user preferences of the application,
      ii. the user's demographic information, and
      iii. the user's engagement with the application.
Embodiment 445: A method of training a model for generating a treatment plan for a user, comprising:
   a. collecting (i) a set of features from an application on a communication device of the user (ii) a profile of the user;
   b. training a first neural network to encode sentiment content from the set of features to determine a marker that is predictive of the user's response to an intervention, thereby providing an encoding of the sentiment content; and
   c. training a second neural network to generate the treatment plan based on the profile of the user.
Embodiment 446: The method of Embodiment 445, wherein the set of features comprises at least two of the following:
   i. voice data;
   ii. textual data, wherein the textual data comprises text and character depicted expression;
   iii. location data;
   iv. application usage data;
   v. biometric data;
   vi. sleep data;
   vii. activity data; and
   viii. self-reported data.
Embodiment 447: The method of Embodiment 445, wherein the first neural network is configured to process missing features in the set of features.
Embodiment 448: The method of Embodiment 445, wherein:
   a. the encoding of the sentiment content (i) discards semantic content from the set of features, and
      (ii) represents sentiment content that provides an indication of a sentiment of the user.
Embodiment 449: The method of Embodiment 445, wherein the profile of the user comprises at least one of the following:
   i. user preferences of the application data;
   ii. user demographic information data; and
   iii. user engagement with the application data.
Embodiment 450: The method of Embodiment 445, wherein the model is trained using a machine learning algorithm selected from principal component analysis, uniform manifold approximation and projection, artificial neural network, time series modeling, or any combination thereof.
Embodiment 451: A method of using a model to generate a treatment plan for a user, comprising:
   a. collecting (i) a set of features from an application on a communication device of a user (ii) a profile of the user;
   b. processing the set of features, using a neural network, to encode sentiment content from the set of features, thereby providing an encoding of the sentiment content;
   c. determining a marker that is predictive of the user's response to an intervention; and
   d. generating a treatment plan based on the profile of the user.
Embodiment 452: The method of Embodiment 451, wherein the set of features comprises at least two of the following:
   i. voice data;
   ii. textual data, wherein the textual data comprises text and character depicted expression;
   iii. location data;
   iv. application usage data;
   v. biometric data;
   vi. sleep data;
   vii. activity data; and
   viii. self-reported data.
Embodiment 453: The method of Embodiment 451, wherein the neural network is configured to process missing features in the set of features.
Embodiment 454: The method of Embodiment 451, wherein:
   a. the encoding of sentiment content discards semantic content from the set of features; and
   b. the encoding of sentiment content provides an indication of a sentiment of the user.
Embodiment 455: The method of Embodiment 451, wherein the profile comprises at least one of the following:
   i. user preferences of the application data;
   ii. user demographic information data; and
   iii. user engagement with the application data.
Embodiment 456: The method of Embodiment 451, wherein the model was trained using a machine learning algorithm selected from principal component analysis, uniform manifold approximation and projection, artificial neural network, time series modeling, and any combination thereof.
Embodiment 457: A system to generate a treatment plan for a user, the system comprising:
   a. one or more processors; and
   b. a memory comprising executable instructions which, when executed by the one or more processors, cause the system to:
      i. collect (1) a set of features from an application on a communication device of a user (2) a profile of the user;
      ii. process the set of features, using a neural network, to encode sentiment content from the set of features to determine a marker, thereby providing an encoding of the sentiment content;
      iii. determine an indication of a sentiment of the user based on the encoding of the sentiment content; and
      iv. generating the treatment plan to the user based on the profile of the user.
Embodiment 458: The system of Embodiment 457, wherein the set of features comprises at least two of the following:
   a. voice data;
   b. textual data, wherein the textual data comprises text and character depicted expression;
   c. location data;
   d. application usage data;
   e. biometric data;
   f. sleep data;
   g. activity data; and
   h. self-reported data.
Embodiment 459: The system of Embodiment 457, wherein the neural network is configured to process missing features in the set of features.
Embodiment 460: The system of Embodiment 457, wherein:
   a. the neural network generates an encoding that discards semantic content from the set of features; and
   b. the marker is predictive of the user's response to an intervention.
Embodiment 461: The system of Embodiment 457, wherein the profile comprises at least one of the following:
   a. user preferences of the application data;
   b. user demographic information data; and
   c. user engagement with the application data.
Embodiment 462: The system of Embodiment 457, wherein the set of features are processed using a machine learning algorithm selected from principal component analysis, uniform manifold approximation and projection, artificial neural network, time series modeling, and any combination thereof.
Embodiment 463: A system to train a model for generating a treatment plan for a user, the system comprising:
   a. one or more processors; and
   b. a memory comprising executable instructions which, when executed by the one or more processors, cause the system to:
      i. collect (1) a set of features from an application on a communication device of a user, (2) a profile of the user and;
      ii. train a first neural network to encode sentiment content from the set of features to determine a marker that is predictive of the user's response to an intervention, thereby providing an encoding of the sentiment content; and
      iii. train a second neural network to generate the treatment plan to the user based on the profile of the user.
Embodiment 464: The system of Embodiment 463, wherein the set of features comprises at least two of the following:
   a. voice data;
   b. textual data, wherein the textual data comprises text and character depicted expression;
   c. location data;
   d. application usage data;
   e. biometric data;
   f. sleep data;
   g. activity data; and
   h. self-reported data.
Embodiment 465: The system of Embodiment 463, wherein the first neural network is configured to process missing features in the set of features.
Embodiment 466: The system of Embodiment 463, wherein:
   a. the encoding of the sentiment content that (i) discards semantic content from the set of features, and (ii) represents sentiment content that provides an indication of a sentiment of the user.
Embodiment 467: The system of Embodiment 463, wherein the profile comprises at least one of
   a. user preferences of the application data;
   b. user demographic information data; and
   c. user engagement with the application data.
Embodiment 468: The system of Embodiment 463, wherein the model is trained using a machine learning algorithm selected from principal component analysis, uniform manifold approximation and projection, artificial neural network, time series modeling, and any combination thereof.

### EXAMPLES

The following illustrative examples are representative of embodiments of the stimulation, systems, and methods described herein and are not meant to be limiting in any way.

### Example 1. Data Collection Through Application by Users

### Design

A pilot program for data collection was conducted with candidates that were all recent high school graduates or current college students aged 18 and older, who speak and read English, and who possessed a newer generation iPhone. Students both with and without a known history of mental illness were included. Students were recruited by student ambassadors, who are students located at a diverse set of colleges and universities across the United States. All data was collected through the app. Students were asked to contribute the following data: baseline family history; daily pulse check on moods; thirty second voice recordings three times a week; weekly surveys covering diet, exercise, and general attitudes, and affects; weekly app usage data; monthly (Time 0, Week 4, Week 8) validated instruments measuring anxiety and depression. The participants engaged with the app for an average duration of eight weeks. Students were able to continue contributing data beyond eight weeks if they desired.

### Methods

Outcomes of interest were clinical and subclinical conditions. Clinical conditions of depression and anxiety were measured using validated instruments at Baseline, Week 4, and Week 8. Subclinical conditions were measured weekly through self-report using questions developed by a trained psychologist to assess loneliness and acceptance. The clinical outcomes are summarized in Table 6.

TABLE 6
Definition of outcomes

| Metric | Definition | Outcomes |
|---|---|---|
| PHQ8 | 8-question measure for assessing depression. | PHQ8 total score, PHQ |
| | Each question uses a 4-point Likert scale from 0 = “Not at | categories: minimal, |
| | All” to 3 = “Nearly Every Day” | mild, moderate, and |
| | | severe. |
| GAD7 | 7-question measure for assessing Generalized Anxiety | GAD7 total score, |
| | Disorder. | GAD7 categories, |
| | Each question uses a 4-point Likert scale from 0 = “Not at | minimal, mild |
| | All” to 3 = “Nearly Every Day” | moderate, and severe. |
| Loneliness | Loneliness, measured by response to the question: “During | Continuous score, |
| | the past seven days, how often have you felt lonely?” | Categories |
| | Scale 1-5 with 1 = “Very Slightly” to 5 “Extremely” | High = 1-2, Low = 3-5 |
| Acceptance | Acceptance, measured weekly as a question of “How | Continuous score, |
| | accepted do you feel by your social group?” | Categories |
| | Scale 1-5 with 1 = “Not at All” to 5 = “Strongly” | Low = 1-2, High = 3-5 |

### Results

Approximately 190 students were invited to participate in the pilot program. A total of ninety-three students accepted the invitation, downloaded the app, and provided at least one day of data. The first cohort of students were invited to use the app in the first week of July. Additional students were invited over time to join through September 7th.

Student engagement with the app included 76.3% of students providing at least 30 days of data and 55.9% providing at least 60 days of data. Of the participants, 76% provided complete information on their family and personal history and were thus eligible for analysis. The pilot population was diverse with respect to gender identity, sexual orientation, religion, family situation, and mental health history. The median number of days the app was used in the July cohort was 61 days, which closely matches the eight weeks of data that users were asked to provide.

Of the ninety-three students who enrolled and provided data, seventy-one provided complete information on their family and personal history and were eligible for this analysis. Students were asked to self-report baseline data on demographics, impairments, family situation, personal and family mental health history, and ACES (Adverse Childhood Experience) score. The pilot population was diverse with respect to gender identity, sexual orientation, religion, family situation, and mental health history. The ACES score appears to be low in this population with a mean of 1.25 and a Q1, Q3, range between 0 and 3. The adverse childhood experiences (ACES) score is low in this population with a mean of 1.25 and a Q1, Q3, range between 0 and 3. Use of substances was consistent with the general young adult student population. Table 7 provides a breakdown of the demographic of the seventy-one students.

TABLE 7
Demographics of Students

| | Students | % of |
|---|---|---|
| Variables | (N = 71) | Students |
| Age (years) | | |
| Mean (SD) | 19.9 (2.38) | |
| Median (Q1, Q3) | 19 (18, 20) | |
| Gender Identity n (%) | | |
| Male | 29 | 41% |
| Female | 37 | 52% |
| Other | 3 | 4% |
| Missing | 2 | 3% |
| Sexual Orientation n (%) | | |
| Heterosexual | 48 | 68% |
| LGBTQ+ | 20 | 28% |
| Missing | 3 | 4% |
| Race/Ethnicity n (%) | | |
| Asian | 18 | 25% |
| Black or African American | 3 | 4% |
| Hispanic, Latino/a/x | 4 | 6% |
| Multiple | 21 | 30% |
| South Asian | 7 | 10% |
| White | 17 | 24% |
| Missing | 1 | 1% |
| Religion n (%) | | |
| Atheist | 6 | 8% |
| Christian | 34 | 48% |
| Hindu/Buddhist | 7 | 10% |
| Jewish | 2 | 3% |
| Muslim | 5 | 7% |
| Other | 2 | 3% |
| Missing | 15 | 21% |
| Impairments n (%) | | |
| Hearing | 1 | 1% |
| Visual | 2 | 3% |
| Mobility | 1 | 1% |
| Neurodiverse | 7 | 10% |
| Learning Disability | 2 | 3% |
| Chronic Health Condition | 3 | 4% |
| Family Situation n (%) | | |
| Adopted | 1 | 1% |
| Foreign National or First-Generation | 19 | 27% |
| Immigrant | | |
| First Generation College Student | 8 | 11% |
| College Athlete | 16 | 23% |
| Gamer | 22 | 31% |
| Mental Health Personal History | | |
| Sought professional help for MH condition | 24 | 34% |
| Diagnosis of MH condition | 15 | 21% |
| Used MH counseling services at school | 11 | 15% |
| Used Academic Counseling at School | 33 | 46% |
| Steady Romantic Partner | | |
| Yes | 23 | 32% |
| Substance Use | | |
| Smoke cigarettes/vaping | | |
| Yes | 4 | 6% |
| Marijuana use | | |
| Yes | 19 | 27% |
| Alcohol Use | | |
| Yes | 31 | 44% |
| Controlled substance | | |
| Yes | 5 | 7% |
| Blackout or Hospitalization within past 30 | | |
| days | | |
| Yes | 0 | 0% |
| Adverse Childhood Experiences | | |
| ACE Score | | |
| Mean (SD) | 1.26 (1.67) | |
| Median (Q1, Q3) | 1 (0, 3) | |

At baseline, sixty-seven students completed the weekly survey in which measures of acceptance and loneliness were collected. Thirty-one students (46%) felt lonely, defined by responses of “moderately,” “quite a bit” and “extremely.” The majority (sixty-one students) (91%) of students felt accepted by their social group defined as neutral to strongly accepted.

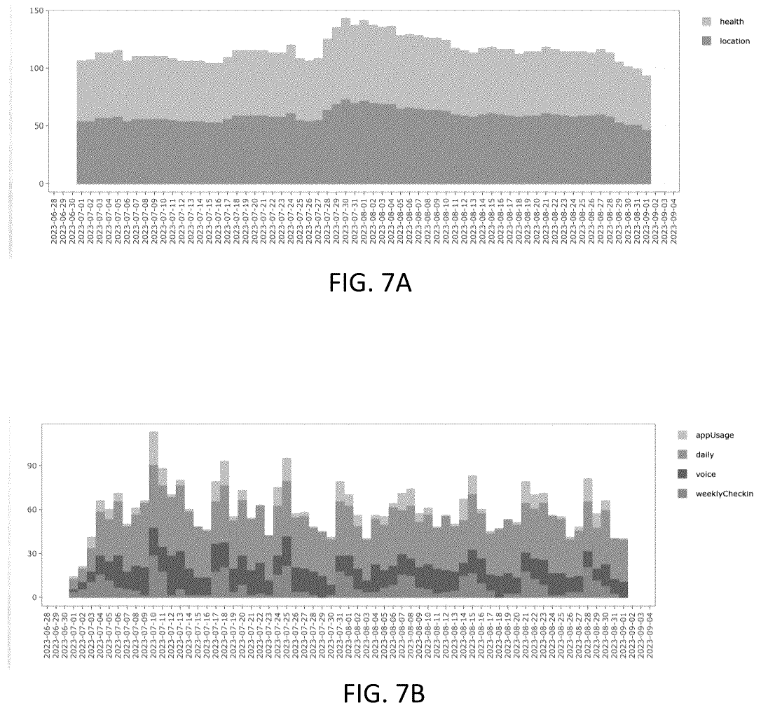

Students provided a variety of data including health data and location data (FIG. 7A) as well as app usage data, daily check-in data, voice data, and weekly check-in data (FIG. 7B). Throughout the program, students were asked to self-report weekly on sleep, meals, exercise, substance use, top of mind, socialization, and physical/biological activity. Daily, students were asked to self-report their emotional state. The app collected over forty streams of passive data in addition to the active data, with specific elements under the broad headings of health, GPS movement, voice markers, app use, etc. Additional data streams, such as weather and local events, were inferred based on location and date. The data was provided at a consistent rate over the course of the eight-week pilot. The overall rate of data contribution was lower for data that required the students to actively participate. The consistent rate of data provided indicates the app is collecting data students are willing to contribute and that data requests represent an acceptable burden to users over time.

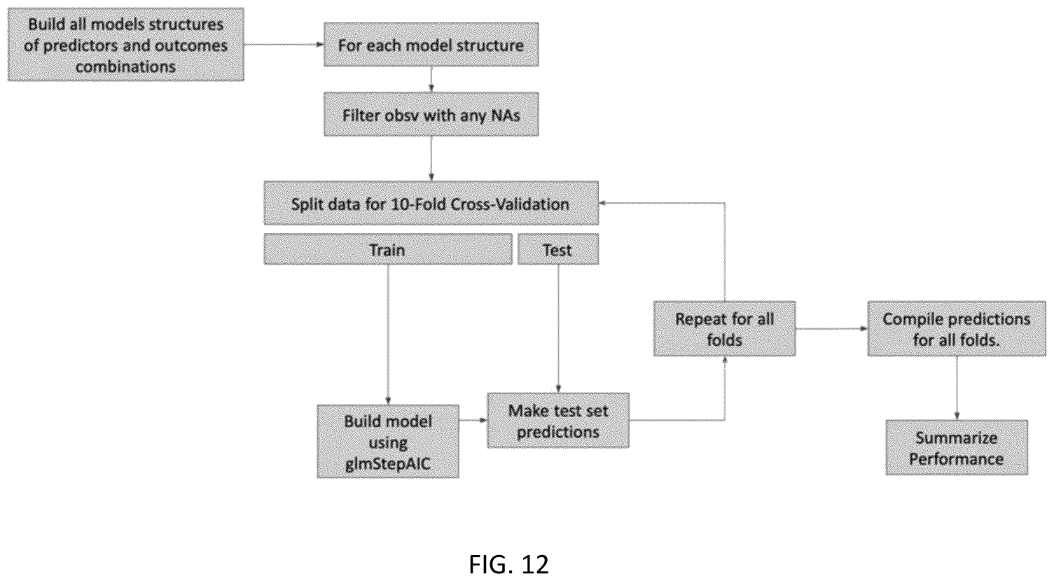

To process the data, a variety of statistical methods were employed within an Al framework (FIG. 12). Voice pipeline: For the voice pipeline the audio segments were parsed into utterances (smallest continuous unit of speech beginning and ending in a clear pause). Features were obtained within each utterance and subsequently averaged over the entire time interval. Features included glottal characteristics that describe how the sound is articulated at the vocal cords and spectral/formant features that describe phonetic quality. App usage pipeline: Screenshots of app usage were parsed into regions defined as collections of neighboring pixels that share a common color with some tolerance. To reduce artifacts, a minimum island size constraint was applied. A python package called EasyOCR was used to capture contents and sizes of text elements to be captured. The string was then parsed and converted into usable features. Multi-language screenshot translation was supported by Google Translate. Clinical features were associated with outcomes using statistical tests without correction for multiple hypothesis testing. When comparing means of prognostic biomarkers between depression, anxiety, loneliness, and acceptance categories, Welch's two-sample t-test was used. When comparing the distribution of categories between dichotomous clinical outcomes described above, a chi-square test was used. Predictors were plotted stratifying by baseline outcome groups over time. Additionally, predictors were summarized based on features collected directly prior to reported outcome measurements and plotted over time. These plots illustrate the direct relationship between both baseline outcomes and future outcomes. Multivariable modeling combining multiple features to predict dichotomous outcomes employed the following: For characteristics associated with dichotomous and ordinal outcomes, samples were filtered for completeness and then split into 10-fold training and testing dataset. The training dataset was used to tune and estimate the model parameters for a log linear model via neural net and predictions were made on the test set. The predictions were compiled across the folds and the performance accuracy, sensitivity, and specificity were estimated with 95% confidence intervals. For features collected over time (e.g. steps, app usage, daily and weekly self report), summary statistics of the days directly prior to the outcome measurement (e.g. mean, sd, %) were used as inputs into the model.

Four base metrics were used to analyze the outcomes. 1) PHQ8, an 8-question measure for assessing depression, 2) GAD7, a 7-question measure for assessing Generalized Anxiety Disorder, 3) Loneliness, measured by response to the question: “During the past seven days, how often have you felt lonely?,” and 4) Acceptance, measured weekly as a question of “How accepted do you feel by your social group?” The markers from the data streams were further analyzed to explore associations between continuous variables and outcomes. When comparing means of prognostic markers between depression, anxiety, loneliness, and acceptance categories, Welch's two-sample t-test was used. When comparing the distribution of categories between dichotomous clinical outcomes described above, a chi-square test was used. Machine learning (ML) modeling was employed to derive features, in particular voice and app usage.

TABLE 8
Statistically significant correlations between baseline characteristics and clinical conditions.

| | Categorize | | | | | |
|---|---|---|---|---|---|---|
| | Depression | | Categorize | Categorize | | |
| | (mild, | | Anxiety | Anxiety | | |
| | moderate, | | (minimal, | (minimal, | | |
| | moderately | Categorize | mild, | mild, | Categorized | Categorized |
| Self- | severe, | Anxiety | moderate, | moderate/ | Loneliness | Acceptance |
| Assessment | severe) | (high, low) | severe) | severe) | (high, low) | (high, low) |
| | | | | | Higher | Lower rates |
| | Higher | Higher | Higher | Higher | rates of | of |
| | rates of | rates of | rates of | rates of | loneliness | acceptance |
| | depression | anxiety are | anxiety are | anxiety are | are | are |
| | associated | associated | associated | associated | associated | associated |
| | with: | with: | with: | with: | with: | with: |
| Category | p-value | | | | | |
| Female | 0.047 | 0.003 | 0.0003 | 0.0001 | | |
| Sexual | 0.031 | | | | | |
| orientation | | | | | | |
| (straight vs. | | | | | | |
| other) | | | | | | |
| Chronic | 0.007 | | | | | 0.028 |
| health | | | | | | |
| condition | | | | | | |
| Marijuana | 0.045 | | | | | |
| use (past 30 | | | | | | |
| days) | | | | | | |
| Neurodiverse | | 0.0006 | 0.0001 | 0.0008 | | |
| Used campus | | 0.004 | 0.004 | 0.003 | 0.017 | |
| mental | | | | | | |
| health | | | | | | |
| resources | | | | | | |
| Alcohol use | | 0.006 | 0.0178 | 0.007 | | |
| (past 30 | | | | | | |
| days) | | | | | | |
| Family | | 0.033 | 0.019 | 0.007 | | |
| history | | | | | | |
| mental | | | | | | |
| illness | | | | | | |
| Previously | | | 0.012 | 0.006 | | |
| sought | | | | | | |
| resources for | | | | | | |
| mental | | | | | | |
| health | | | | | | |
| Diagnosed | | | 0.012 | 0.04 | | |
| with mental | | | | | | |
| illness | | | | | | |
| Stimulant | | | 0.028 | 0.05 | | |
| use (past 30 | | | | | | |
| days) | | | | | | |
| Playing a | | | | 0.042 | | |
| college sport | | | | | | |

Generalized anxiety disorder (GAD) is a prevalent psychiatric condition, characterized by excessive worry about everyday life events. GAD is often accompanied by symptoms such as hyperarousal, autonomic hyperactivity, irritability, sleep disturbances, and muscle tension. Screening for GAD is recommended in adults aged 19 to 65.

The GAD-7, is a 7-item anxiety scale, is an effective tool for identifying and assessing GAD in clinical practice. The GAD-7 exhibits strong criterion validity for identifying probable GAD cases and serves as a reliable severity measure, with higher scores correlating significantly with functional impairment and disability. Factor analysis confirms the distinction between GAD and depression as separate dimensions, even though there is a known comorbidity between anxiety and depressive disorders. A score of 10 or greater on the GAD-7 has been identified as a reasonable threshold for GAD diagnosis, with lower scores indicating minimal to mild anxiety levels. The scale is particularly useful for tracking symptom severity and change over time.

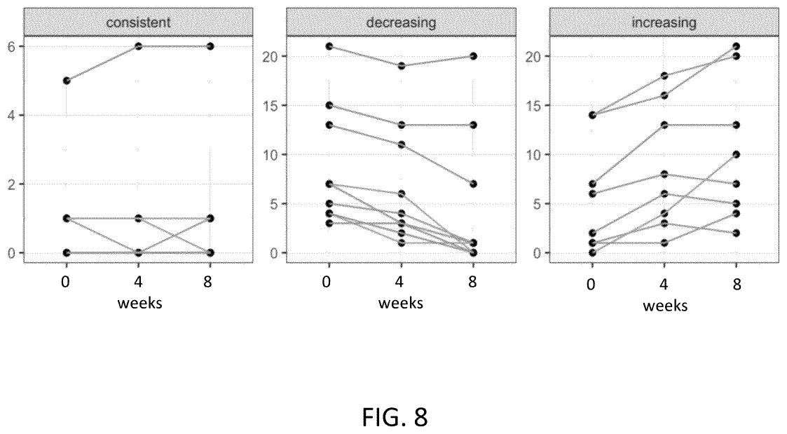

At baseline, sixty-five students completed the GAD-7. Thirteen (210%) students had anxiety classified as moderate to moderately-severe. GAD-7 was also assessed at Week 4 and Week 8. The mean GAD-7 score for the population increased slightly from 5.5 at Baseline to 6.3 at Week 8. Group changes in GAD-7 were relatively modest, however some users showed marked changes in their levels of anxiety. FIG. 8 depicts changes in GAD-7 scores in user users throughout the course of the pilot for users who have completed the eight-week pilot.

Depression is highly prevalent in primary care settings, with many patients remaining undiagnosed. Patients with depression may exhibit various symptoms, making diagnosis particularly challenging when somatic symptoms are present.

The PHQ-9 is a brief measure of depression severity. There is strong evidence for the PHQ-9's validity, including criterion validity, construct validity, and external validity, based on data from two studies involving a total of 6,000 patients. PHQ-9's can establish different levels of depression severity, with scores of 5, 10, 15, and 20 serving as thresholds for mild, moderate, moderately severe, and severe depression. While there are various measures available for identifying depression, the PHQ-9 stands out due to its brevity, exclusive focus on DSM-IV diagnostic criteria.

The PHQ-8 (Patient Health Questionnaire-8) is a reduced version of PHQ-9, with the last question on suicide ideation removed. The PHQ8 has been validated using the same cut points for depression severity as PHQ-9. The population prevalence of depression detected by the PHQ-8 is aligned with rates reported in other population-based studies.

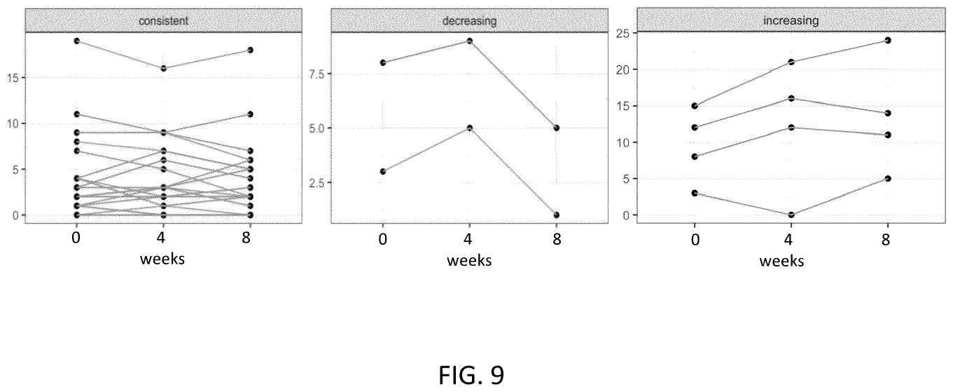

At baseline, sixty-five students completed the PHQ8 surveys for depression. Thirteen (21%) students had depression classified as moderate to moderately-severe depression. PHQ8 was also assessed at Week 4 and Week 8. The mean result for PHQ8 remained stable throughout the first 8 weeks but then dipped by Week 12 from 5.6, 5.1, 5.3 to 4.2 at baseline, Week 4, 8 and 12, respectively. (FIG. 9).

The association between baseline characteristics and baseline clinical conditions was evaluated. When depression was categorized as high versus low, the following characteristics were significantly associated with higher rates of depression: higher adverse childhood experiences score (ACES) (p=0.047), female (p=0.027), and use of prescription drugs not prescribed (p=0.053).

When anxiety was categorized as high versus low, the following characteristics were significantly associated with higher rates of anxiety: female (p=0.005), neurodiverse (p=0.003), having been diagnosed with a mental health condition (p=0.035), using mental health counseling services provided by school (p=0.001), and use of alcohol (p=0.006).

When loneliness was categorized as yes versus no, although a large proportion (46%) of students reported feeling lonely, no user characteristics were significantly associated with increasing feelings of loneliness.

When acceptance was categorized as yes versus no, the following characteristics were significantly associated with higher rates of not feeling accepted: higher ACES (p=0.009), female (0.054), and having a chronic health condition (p=0.012).

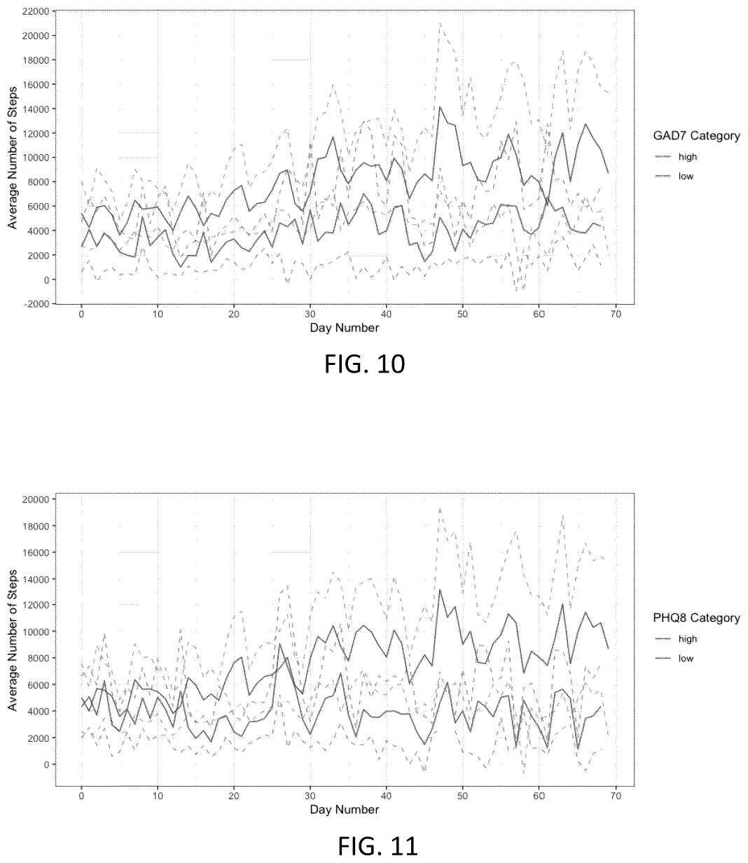

Step counts data were extracted from the broader wearables data. Average number of steps per day was compared between groups of users with low and high GAD7 (FIG. 10) and PHQ8 (FIG. 11). Users who had higher levels of anxiety and depression had consistently lower step counts than users with lower levels of anxiety and depression.

The association between baseline characteristics and baseline clinical conditions were evaluated. The correlations that showed statistical significance are shown in Table 8. High complexity input data streams were turned into derived features for use in modeling. Specifically the following features were derived and used in statistical modeling:

Step count & anxiety/depression: Users who have higher levels of anxiety and depression (from GAD7 and PHQ8) have consistently lower step counts than those with lower levels of anxiety and depression.

Phone use & acceptance: Students who have low acceptance at baseline tended to use their phone via various apps more frequently than those who felt accepted.

Sleep & depression: Sleep observations are more variable for those reporting high depression versus low or no depression.

Activeness & depression: Students who have low depression at baseline tended to be more active, having more steps compared to those who have high depression.

Phone use & anxiety: Students who have anxiety.

Steps & anxiety: Students who have low anxiety at baseline tended to be more active, having more steps compared to those who have high anxiety.

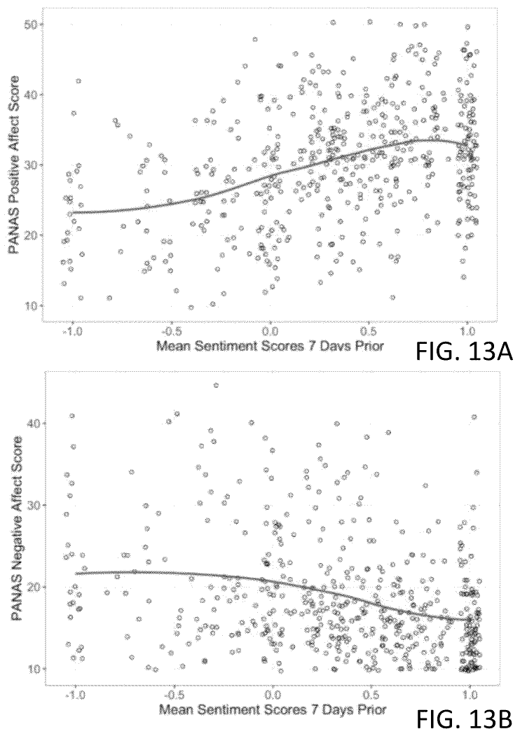

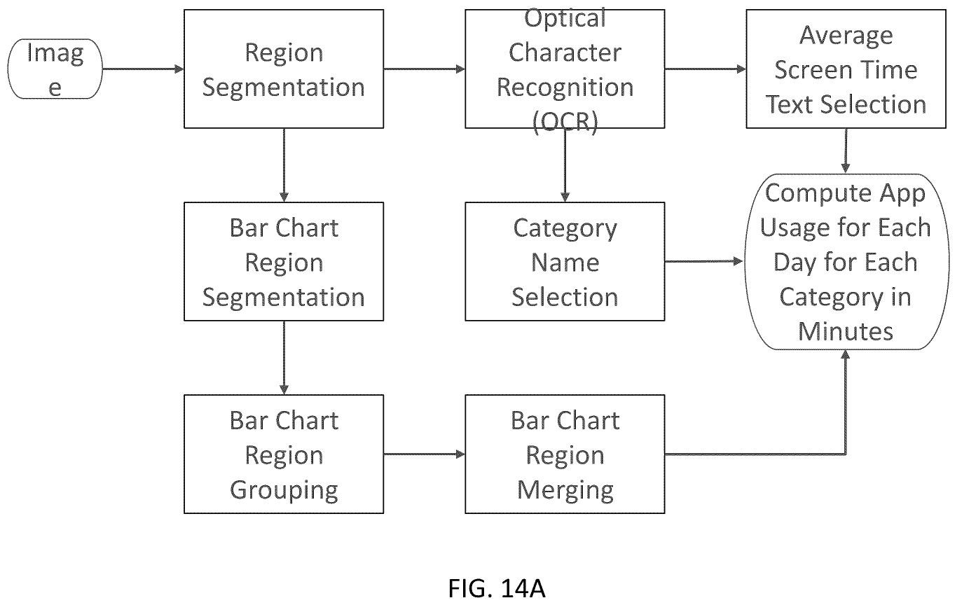

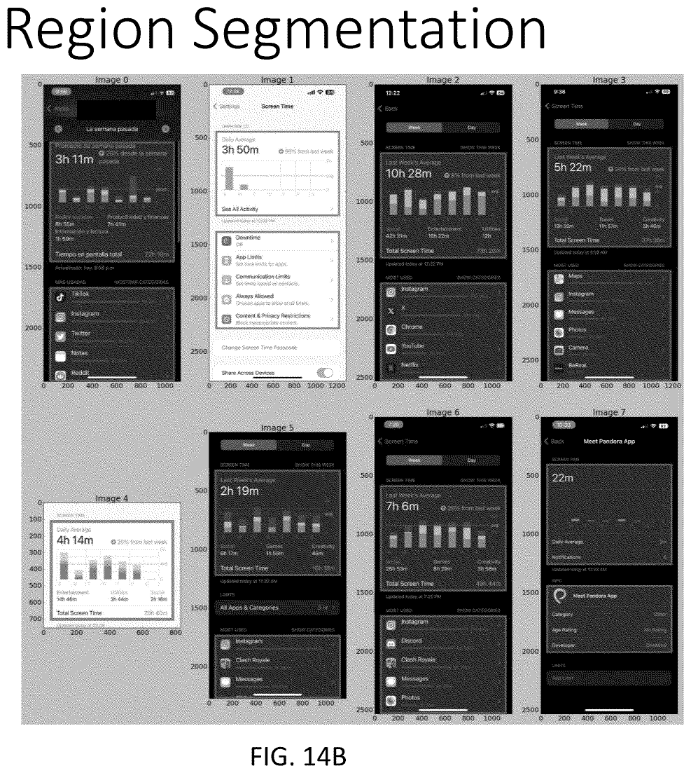

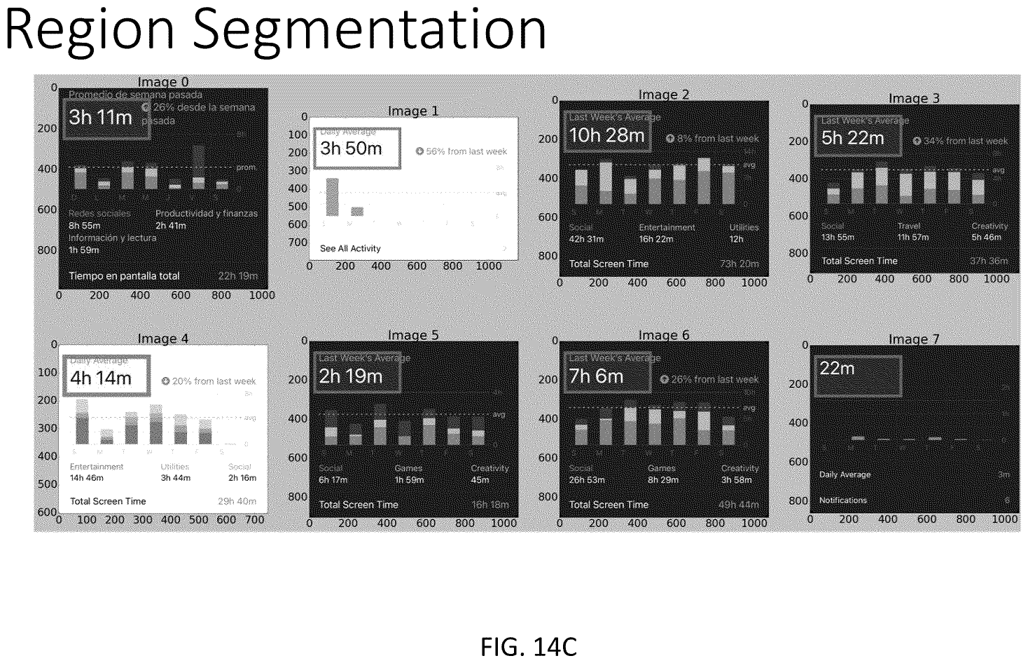

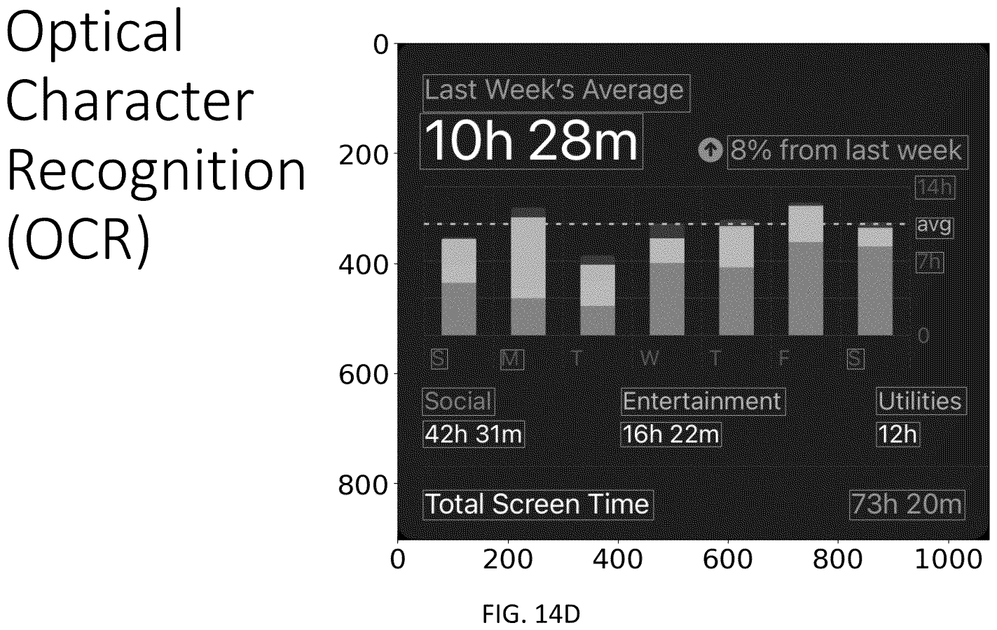

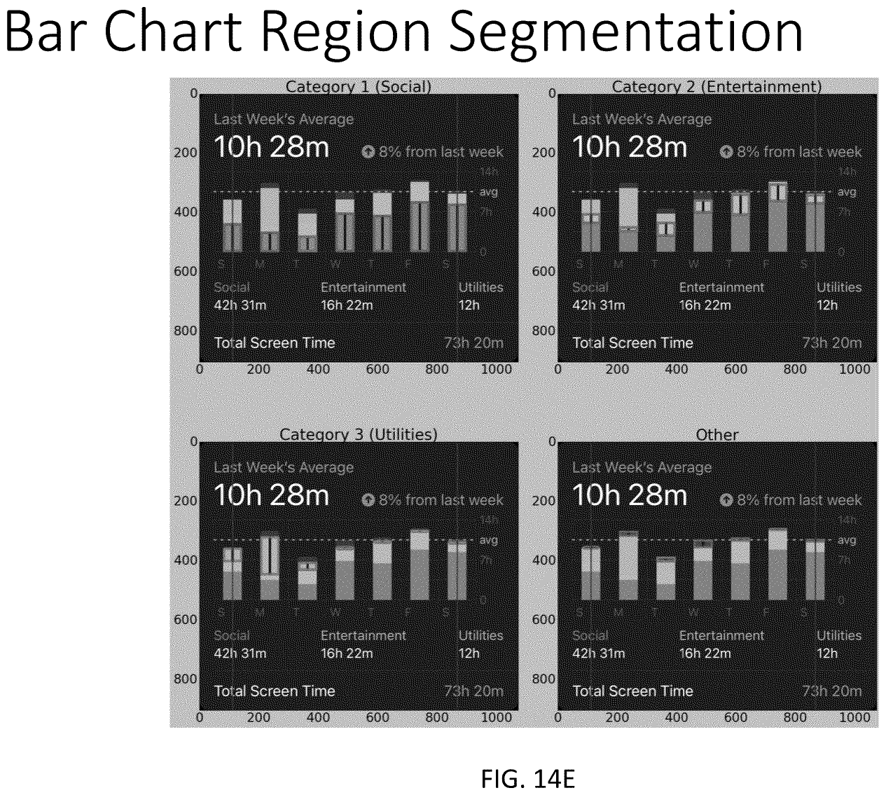

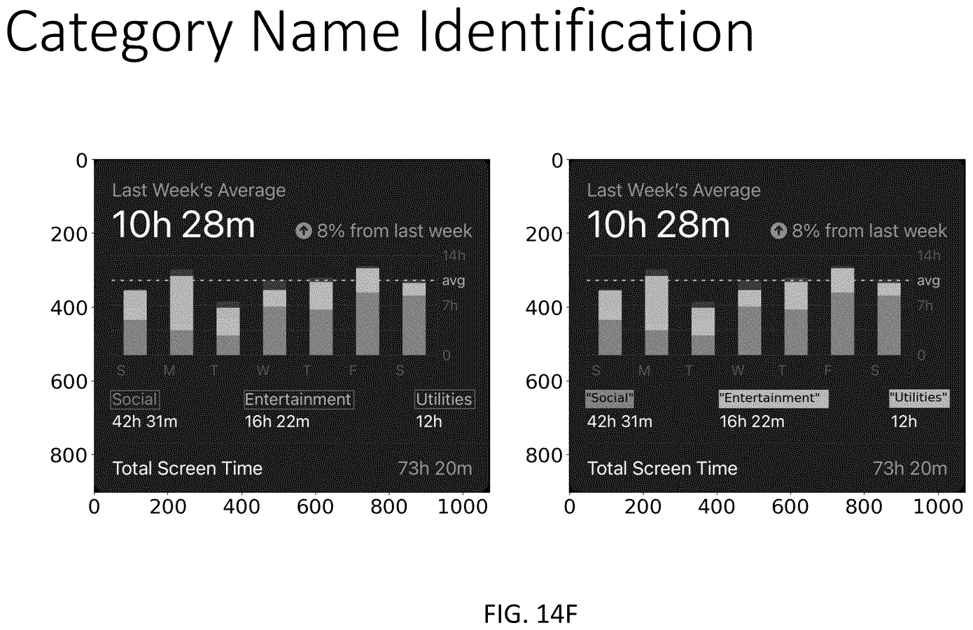

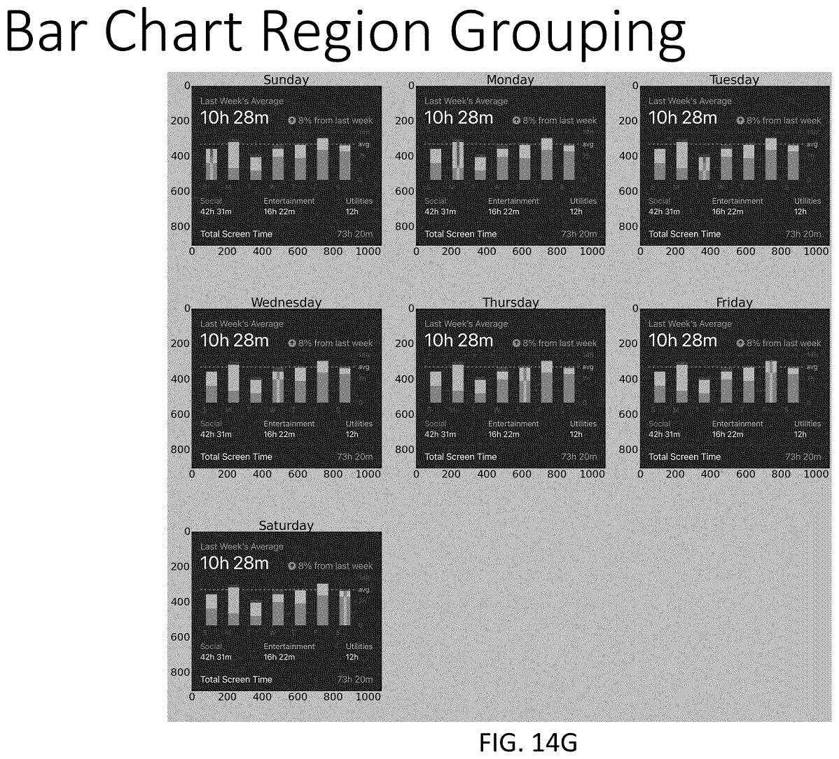

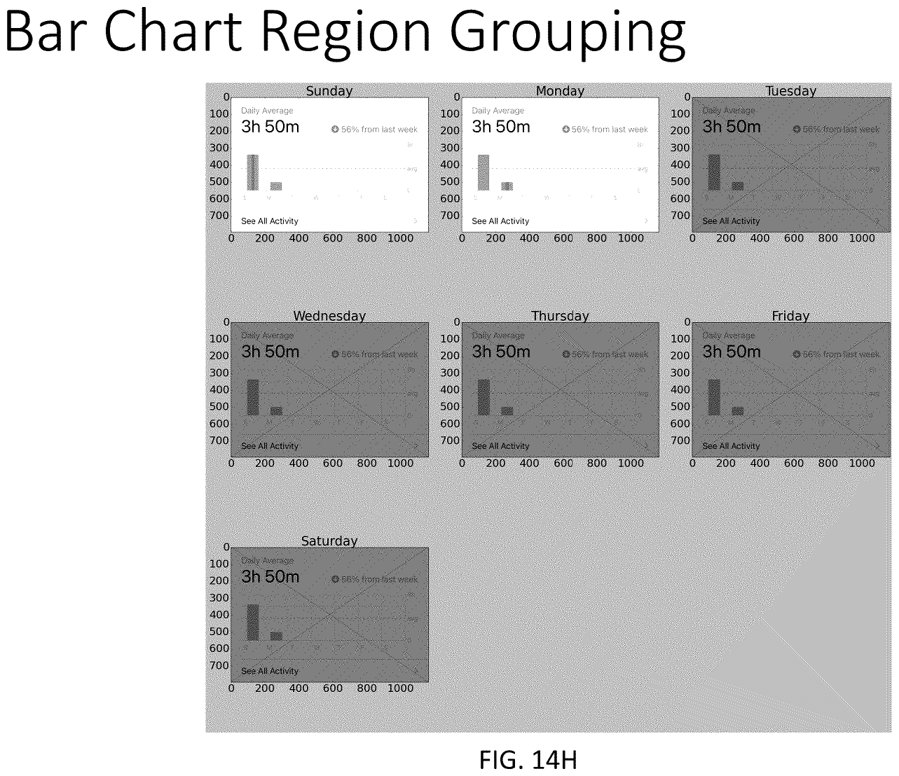

Emojis & PANAS. We examined the internal consistency of responses by comparing the daily emoji reports with the validated PANAS instrument (FIGS. 13A-13B). The emojis were converted to a sentiment score and the results suggest that it may be possible to replace the PANAS survey with a daily input emoji to reduce user burden.

### Example 2. Data Aggregation and Analysis of Current Digital Markers

The data collected needs to be formatted, labeled, and cleaned prior to implementation in machine learning. The next step is to derive features from the raw data. For example, a data stream can be a voice stream. A student-provided voice clip is characterized by duration, number of words per minute, number of utterances per minute, and whether the prompt they chose to answer should elicit a positive or neutral reaction. Each utterance, defined as a continuous unit of speech beginning and ending with a pause, is further characterized by 20+ features describing vocal characteristics (e.g., glottal open closure (speech characteristic (e.g., spectral, tempo, etc.), and background noise (level, etc.). In a similar fashion to voice, features will be derived for each data stream collected (e.g., GPS, app usage, health data, daily and weekly emotions, and baseline characteristics). A larger data set can support more diversity to yield a more expansive feature set. This occurrence can provide deeper insight into the impact of culturally diverse populations on mental and behavioral health, and that knowledge could be used to further sub divide sub-clinical populations and cross correlate broader behavioral trends.

### Example 3. AI/ML Model Generation and Mental Health Flag Development

Use of data to strengthen currently identified correlations, identify new digital markers correlations that were not present in the original cohort studied, and build more predictive models will be performed. Initial analysis identified several predictive digital markers. These markers have been combined using ML methods such as PCA and statistical models such as logistic regression, resulting in multimodal data models that predict clinical and subclinical mental health status. These models are limited by both the number of users evaluated and the time for which they are followed. With the larger number of students and longer follow-up time, it is expected to observe changes in student's mental health such that more robust modeling can be done. The results of a vastly increased study will be taken to tune and adjust the input and output parameters of the machine learning model. This will represent a critical step in creating a robust and reliable platform that can interpret results for users based on aggregate behavior analyzed by sub-clinical populations. The strength of the ML/AI to provide individualized results for unique subsets of students is reliant on processing and implementing a curated and optimized data set.

To understand the interactive nature of the various markers, a multivariate analysis technique will be applied to further examine the relationship between variables, including dimension reduction methods (e.g., principal components analysis, long-short-term memory networks, or autoencoders), outlier analysis, item reliability, and prediction modeling (e.g., LASSO/elastic net regression or neural nets). The models are expected to identify at least three additional subclinical and clinical indicators of mental health.

Significant marker associations will be used to translate into flags for the app. This will involve weighing the importance of the marker(s) and associated cross-corollary data to interpret the user flag in the broader context for the user's mental health. For example, rather than provide a student a statistical result, results for each condition may be expressed on a 1-100 scale where 100 indicates no signs of developing a condition and a score of one represents substantial changes in behavior consistent with a condition. Development of at least five additional mental health flags for sub-clinical or clinical conditions is expected.

The larger data set, and tuned parameters will not only lead to the discovery of new digital behaviors that indicate mental health status. These markers not only add to the ability to flag potential declines in mental health but add to the wealth of knowledge on the conscious and unconscious behaviors that reflect the mental state of college students.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

We claim:

1. A method of training a model for generating a treatment plan for a user, comprising:
(a) collecting a set of features from an application on a communication device of a user;
(b) training a first neural network to encode sentiment content from the set of features to determine a marker that is predictive of the user's response to an intervention; and
(c) training a second neural network to generate a treatment plan based on a profile of the user.

2. The method of claim 1, wherein the set of features comprises at least two of the following:
(i) voice data;
(ii) textual data, wherein the textual data comprises text and character depicted expression;
(iii) location data;
(iv) application usage data;
(v) biometric data;
(vi) sleep data;
(vii) activity data; and
(viii) self-reported data.

3. The method of claim 1, wherein the first neural network is configured to process missing features in the set of features.

4. The method of claim 1, wherein:
the first neural network generates an encoding that (i) discards semantic content from the set of features, and (ii) represents sentiment content that provides an indication of a sentiment of the user.

5. The method of claim 1, wherein the user's profile comprises at least one of the following:
(i) the user's preferences of the application;
(ii) the user's demographic information; and
(iii) the user's engagement with the application.

6. The method of claim 1, wherein the model is trained using a machine learning algorithm selected from principal component analysis, uniform manifold approximation and projection, artificial neural network, time series modeling, and any combination thereof.

7. A method of using a model to generate a treatment plan for a user, comprising:
(a) collecting a set of features from an application on a communication device of a user;
(b) processing the set of features, using a neural network, to encode sentiment content from the set of features;
(c) determining a marker that is predictive of the user's response to an intervention; and
(d) generating a treatment plan based on a profile of the user.

8. The method of claim 7, wherein the set of features comprises at least two of the following:
(i) voice data;
(ii) textual data, wherein the textual data comprises text and character depicted expression;
(iii) location data;
(iv) application usage data;
(v) biometric data;
(vi) sleep data;
(vii) activity data; and
(viii) self-reported data.

9. The method of claim 7, wherein the neural network is configured to process missing features in the set of features.

10. The method of claim 7, wherein:
the encoding discards semantic content from the set of features; and
the encoded sentiment content provides an indication of a sentiment of the user.

11. The method of claim 7, wherein the profile comprises at least one of the following:
(i) the user's preferences of the application;
(ii) the user's demographic information; and
(iii) the user's engagement with the application.

12. The method of claim 7, wherein the model was trained using a machine learning algorithm selected from principal component analysis, uniform manifold approximation and projection, artificial neural network, time series modeling, and any combination thereof.

13. A system to generate a treatment plan for a user, the system comprising:
(a) one or more processors; and
(b) a memory comprising executable instructions which, when executed by the one or more processors, cause the system to:
(i) collect a set of features from an application on a communication device of a user;
(ii) process the set of features, using a neural network, to encode sentiment content from the set of features to determine a marker;
(iii) determine an indication of a sentiment of the user based on the encoded sentiment content; and
(iv) generate a treatment plan to the user based on a profile of the user.

14. The system of claim 13, wherein the set of features comprises at least two of the following:
(A) voice data;
(B) textual data, wherein the textual data comprises text and character depicted expression;
(C) location data;
(D) application usage data;
(E) biometric data;
(F) sleep data;
(G) activity data; and
(H) self-reported data.

15. The system of claim 13, wherein the neural network is configured to process missing features in the set of features.

16. The system of claim 13, wherein:
the neural network generates an encoding that discards semantic content from the set of features; and
the marker is predictive of the user's response to an intervention.

17. The system of claim 13, wherein the profile comprises at least one of the following:
(A) the user's user preferences of the application;
(B) the user's demographic information; and
(C) the user's engagement with the application.

18. The system of claim 13, wherein the set of features are processed using a machine learning algorithm selected from principal component analysis, uniform manifold approximation and projection, artificial neural network, time series modeling, and any combination thereof.

19. A system to generate a treatment plan to a user, the system comprising:
(a) one or more processors; and
(b) a memory comprising executable instructions which, when executed by the one or more processors, cause the system to:
(i) collect a set of features from an application on a communication device of a user;
(ii) train a first neural network to encode sentiment content from the set of features to determine a marker that is predictive of the user's response to an intervention; and
(iii) train a second neural network to generate a treatment plan to the user based on a profile of the user.

20. The system of claim 19, wherein the set of features comprises at least two of the following:
(A) voice data;
(B) textual data, wherein the textual data comprises text and character depicted expression;
(C) location data;
(D) application usage data;
(E) biometric data;
(F) sleep data;
(G) activity data; and
(H) self-reported data.

21. The system of claim 19, wherein the first neural network is configured to process missing features in the set of features.

22. The system of claim 19, wherein:
the first neural network generates an encoding that (i) discards semantic content from the set of features, and (ii) represents sentiment content that provides an indication of a sentiment of the user.

23. The system of claim 19, wherein the profile comprises at least one of
(A) the user's preferences of the application;
(B) the user's demographic information; and
(C) the user's engagement with the application.

24. The system of claim 19, wherein the model is trained using a machine learning algorithm selected from principal component analysis, uniform manifold approximation and projection, artificial neural network, time series modeling, and any combination thereof.
